SEQUENCE LISTING (99 sequences)

SEQ ID NO: 1 - protein, 15 aa, Artificial Sequence
  Synthetic Peptide
KCQVLLSNPTSGCSW

SEQ ID NO: 2 - protein, 10 aa, Artificial Sequence
  Synthetic Peptide
SQFRVSPLDR

SEQ ID NO: 3 - protein, 10 aa, Artificial Sequence
  Synthetic Peptide
VLLSNPTSGC

SEQ ID NO: 4 - protein, 20 aa, Artificial Sequence
  Synthetic Peptide
KAAEGLDTQRFSGKRLGDTF

SEQ ID NO: 5 - protein, 16 aa, Artificial Sequence
  Synthetic Peptide
FSGKRLGDTFVLTLSD

SEQ ID NO: 6 - protein, 16 aa, Artificial Sequence
  Synthetic Peptide
SALSNSIMYFSHFVPV

SEQ ID NO: 7 - protein, 13 aa, Artificial Sequence
  Synthetic Peptide
FQPRGAAASPTFL

SEQ ID NO: 8 - protein, 11 aa, Artificial Sequence
  Synthetic Peptide
LYLSQNKPKAA

SEQ ID NO: 9 - protein, 5 aa, Artificial Sequence
  Synthetic Peptide
  D-amino acid (1)..(5)
HSFVT

SEQ ID NO: 10 - protein, 5 aa, Artificial Sequence
  Synthetic Peptide
  D-amino acid (1)..(5)
KDNSN

SEQ ID NO: 11 - protein, 5 aa, Artificial Sequence
  Synthetic Peptide
  D-amino acid (1)..(5)
RTNNH

SEQ ID NO: 12 - protein, 5 aa, Artificial Sequence
  Synthetic Peptide
  D-amino acid (1)..(5)
EANDR

SEQ ID NO: 13 - protein, 5 aa, Artificial Sequence
  Synthetic Peptide
  D-amino acid (2)..(5)
GLENR

SEQ ID NO: 14 - protein, 5 aa, Artificial Sequence
  Synthetic Peptide
  D-amino acid (1)..(4)
NNRVG

SEQ ID NO: 15 - protein, 5 aa, Artificial Sequence
  Synthetic Peptide
  D-amino acid (1)..(5)
EYEYV

SEQ ID NO: 16 - protein, 5 aa, Artificial Sequence
  Synthetic Peptide
  D-amino acid (2)..(5)
GWDPN

SEQ ID NO: 17 - protein, 5 aa, Artificial Sequence
  Synthetic Peptide
  D-amino acid (1)..(5)
DWFSN

SEQ ID NO: 18 - protein, 5 aa, Artificial Sequence
  Synthetic Peptide
  D-amino acid (1)..(5)
KKLWA

SEQ ID NO: 19 - protein, 5 aa, Artificial Sequence
  Synthetic Peptide
  D-amino acid (1)..(5)
WPHTV

SEQ ID NO: 20 - protein, 5 aa, Artificial Sequence
  Synthetic Peptide
  D-amino acid (1)..(2)
  D-amino acid (4)..(5)
TEGWF

SEQ ID NO: 21 - protein, 5 aa, Artificial Sequence
  Synthetic Peptide
  D-amino acid (1)..(5)
TFPKK

SEQ ID NO: 22 - protein, 11 aa, Artificial Sequence
  Synthetic peptide
  Xaa (1)..(1): Xaa = an azide-containing amino acid residue with four carbons in the azide-terminated side chain
XSQFRVSPLDR

SEQ ID NO: 23 - protein, 10 aa, Artificial Sequence
  Synthetic Peptide
  Xaa (1)..(1): Xaa = an azide-containing amino acid residue with four carbons in the azide-terminated side chain
XVLLSNPTSG

SEQ ID NO: 24 - protein, 20 aa, Artificial Sequence
  Synthetic Peptide
  Xaa (10)..(10): Xaa = azide-containing amino acid residue with four carbons in the azide-terminated side chain
KAAEGLDTQXFSGKRLGDTF

SEQ ID NO: 25 - protein, 16 aa, Artificial Sequence
  Synthetic Peptide
  Xaa (7)..(7): Xaa = azide-containing amino acid residue with four carbons in the azide-terminated side chain
FSGKRLXDTFVLTLSD

SEQ ID NO: 26 - protein, 16 aa, Artificial Sequence
  Synthetic Peptide
  Xaa (8)..(8): Xaa = azide-containing amino acid residue with four carbons in the azide-terminated side chain
SALSNSIXYFSHFVPV

SEQ ID NO: 27 - protein, 5 aa, Artificial Sequence
  Synthetic Peptide
  D-amino acid (1)..(5)
KRSAH

SEQ ID NO: 28 - protein, 5 aa, Artificial Sequence
  Synthetic Peptide
  D-amino acid (1)..(5)
SNPRK

SEQ ID NO: 29 - protein, 5 aa, Artificial Sequence
  Synthetic Peptide
  D-amino acid (1)..(5)
NFPKK

SEQ ID NO: 30 - protein, 5 aa, Artificial Sequence
  Synthetic Peptide
  D-amino acid (1)..(5)
VLYRR

SEQ ID NO: 31 - protein, 5 aa, Artificial Sequence
  Synthetic Peptide
  D-amino acid (1)..(5)
YRPFY

SEQ ID NO: 32 - protein, 5 aa, Artificial Sequence
  Synthetic Peptide
  D-amino acid (1)..(5)
NFYRR

SEQ ID NO: 33 - protein, 5 aa, Artificial Sequence
  Synthetic Peptide
  D-amino acid (1)..(5)
YFRSR

SEQ ID NO: 34 - protein, 5 aa, Artificial Sequence
  Synthetic Peptide
  D-amino acid (1)..(5)
YRSNY

SEQ ID NO: 35 - protein, 5 aa, Artificial Sequence
  Synthetic Peptide
  D-amino acid (1)..(5)
RPYAY

SEQ ID NO: 36 - protein, 5 aa, Artificial Sequence
  Synthetic Peptide
  D-amino acid (1)..(5)
AYKFN

SEQ ID NO: 37 - protein, 5 aa, Artificial Sequence
  Synthetic Peptide
  D-amino acid (1)..(5)
RFTAF

SEQ ID NO: 38 - protein, 5 aa, Artificial Sequence
  Synthetic Peptide
HGSYG

SEQ ID NO: 39 - protein, 5 aa, Artificial Sequence
  Synthetic Peptide
KRLGA

SEQ ID NO: 40 - protein, 5 aa, Artificial Sequence
  Synthetic Peptide
AKYRG

SEQ ID NO: 41 - protein, 5 aa, Artificial Sequence
  Synthetic Peptide
  D-amino acid (1)..(5)
HALLW

SEQ ID NO: 42 - protein, 5 aa, Artificial Sequence
  Synthetic Peptide
  D-amino acid (1)..(2)
  D-amino acid (4)..(5)
LRGYW

SEQ ID NO: 43 - protein, 5 aa, Artificial Sequence
  Synthetic Peptide
  D-amino acid (1)..(5)
VASHF

SEQ ID NO: 44 - protein, 5 aa, Artificial Sequence
  Synthetic Peptide
  D-amino acid (1)..(1)
  D-amino acid (3)..(5)
NGNVH

SEQ ID NO: 45 - protein, 5 aa, Artificial Sequence
  Synthetic Peptide
  D-amino acid (1)..(5)
WPLRF

SEQ ID NO: 46 - protein, 5 aa, Artificial Sequence
  Synthetic Peptide
  D-amino acid (1)..(5)
RWFNV

SEQ ID NO: 47 - protein, 5 aa, Artificial Sequence
  Synthetic Peptide
  D-amino acid (1)..(5)
HAVWH

SEQ ID NO: 48 - protein, 5 aa, Artificial Sequence
  Synthetic Peptide
  D-amino acid (1)..(5)
WVPLW

SEQ ID NO: 49 - protein, 5 aa, Artificial Sequence
  Synthetic Peptide
  D-amino acid (1)..(5)
FFRLY

SEQ ID NO: 50 - protein, 5 aa, Artificial Sequence
  Synthetic Peptide
  D-amino acid (1)..(3)
  D-amino acid (5)..(5)
WYYGF

SEQ ID NO: 51 - protein, 5 aa, Artificial Sequence
  Synthetic Peptide
AGDSW

SEQ ID NO: 52 - protein, 5 aa, Artificial Sequence
  Synthetic Peptide
HVRHG

SEQ ID NO: 53 - protein, 5 aa, Artificial Sequence
  Synthetic Peptide
HGRGH

SEQ ID NO: 54 - protein, 5 aa, Artificial Sequence
  Synthetic Peptide
THPTT

SEQ ID NO: 55 - protein, 5 aa, Artificial Sequence
  Synthetic Peptide
FAGYH

SEQ ID NO: 56 - protein, 5 aa, Artificial Sequence
  Synthetic Peptide
WTEHG

SEQ ID NO: 57 - protein, 5 aa, Artificial Sequence
  Synthetic Peptide
PWTHG

SEQ ID NO: 58 - protein, 5 aa, Artificial Sequence
  Synthetic Peptide
TNDFD

SEQ ID NO: 59 - protein, 5 aa, Artificial Sequence
  Synthetic Peptide
LFPFD

SEQ ID NO: 60 - protein, 5 aa, Artificial Sequence
  Synthetic Peptide
  D-amino acid (1)..(4)
SLRFG

SEQ ID NO: 61 - protein, 5 aa, Artificial Sequence
  Synthetic Peptide
  D-amino acid (1)..(3)
  D-amino acid (5)..(5)
YFRGS

SEQ ID NO: 62 - protein, 5 aa, Artificial Sequence
  Synthetic Peptide
  D-amino acid (1)..(4)
WNWVG

SEQ ID NO: 63 - protein, 5 aa, Artificial Sequence
  Synthetic Peptide
  D-amino acid (1)..(4)
VAWLG

SEQ ID NO: 64 - protein, 5 aa, Artificial Sequence
  Synthetic Peptide
  D-amino acid (1)..(4)
FHVHG

SEQ ID NO: 65 - protein, 5 aa, Artificial Sequence
  Synthetic Peptide
  D-amino acid (1)..(5)
WVSNV

SEQ ID NO: 66 - protein, 5 aa, Artificial Sequence
  Synthetic Peptide
  D-amino acid (1)..(5)
WSVNV

SEQ ID NO: 67 - protein, 5 aa, Artificial Sequence
  Synthetic Peptide
  D-amino acid (1)..(4)
LNSHG

SEQ ID NO: 68 - protein, 5 aa, Artificial Sequence
  Synthetic Peptide
  D-amino acid (1)..(1)
  D-amino acid (4)..(5)
YGGVR

SEQ ID NO: 69 - protein, 5 aa, Artificial Sequence
  Synthetic Peptide
  D-amino acid (1)..(4)
NSVHG

SEQ ID NO: 70 - protein, 5 aa, Artificial Sequence
  Synthetic Peptide
  D-amino acid (1)..(4)
TTVHG

SEQ ID NO: 71 - protein, 5 aa, Artificial Sequence
  Synthetic Peptide
  D-amino acid (1)..(3)
  D-amino acid (5)..(5)
FDVGH

SEQ ID NO: 72 - protein, 5 aa, Artificial Sequence
  Synthetic Peptide
  D-amino acid (1)..(2)
  D-amino acid (4)..(5)
RHGWK

SEQ ID NO: 73 - protein, 5 aa, Artificial Sequence
  Synthetic Peptide
  D-amino acid (2)..(5)
GHTWP

SEQ ID NO: 74 - protein, 5 aa, Artificial Sequence
  Synthetic Peptide
  D-amino acid (1)..(1)
  D-amino acid (3)..(3)
  D-amino acid (5)..(5)
HGRGH

SEQ ID NO: 75 - protein, 161 aa, Homo sapiens
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 76 - protein, 169 aa, Mus musculus
KPQAPELRIFPKKMDAELGQKVDLVCEVLGSVSQGCSWLFQNSSSKLPQPTFVVYMASSHNKITWDEKLNSSKLFSAMRDTNNKYVLTLNKFSKENEGYYFCSVISNSVMYFSSVVPVLQKVNSTTTKPVLRTPSPVHPTGTSQPQRPEDCRPRGSVKGTGLDFACDIY

SEQ ID NO: 77 - protein, 19 aa, Artificial Sequence
  Synthetic Peptide
TKKSPSPEFSGMPRISKLG

SEQ ID NO: 78 - protein, 22 aa, Artificial Sequence
  Synthetic Peptide
CPADYFAPGVKSYPDGWNLPGC

SEQ ID NO: 79 - protein, 25 aa, Artificial Sequence
  Synthetic Peptide
CGSAPPDSSWRGSLKVPYNVGPGFC

SEQ ID NO: 80 - protein, 24 aa, Artificial Sequence
  Synthetic Peptide
GCPADYFAPGVKSYPDGWNLPGCG

SEQ ID NO: 81 - protein, 27 aa, Artificial Sequence
  Synthetic Peptide
GCGSAPPDSSWRGSLKVPYNVGPGFCG

SEQ ID NO: 82 - protein, 5 aa, Artificial Sequence
  Synthetic Peptide
  D-amino acid (1)..(5)
KHYTN

SEQ ID NO: 83 - protein, 5 aa, Artificial Sequence
  Synthetic Peptide
  D-amino acid (1)..(5)
NSPRW

SEQ ID NO: 84 - protein, 5 aa, Artificial Sequence
  Synthetic Peptide
  D-amino acid (1)..(5)
HERLK

SEQ ID NO: 85 - protein, 5 aa, Artificial Sequence
  Synthetic Peptide
  D-amino acid (1)..(5)
AHYTN

SEQ ID NO: 86 - protein, 5 aa, Artificial Sequence
  Synthetic Peptide
  D-amino acid (1)..(5)
KAYTN

SEQ ID NO: 87 - protein, 5 aa, Artificial Sequence
  Synthetic Peptide
  D-amino acid (1)..(5)
KHATN

SEQ ID NO: 88 - protein, 5 aa, Artificial Sequence
  Synthetic Peptide
  D-amino acid (1)..(5)
KHYAN

SEQ ID NO: 89 - protein, 5 aa, Artificial Sequence
  Synthetic Peptide
  D-amino acid (1)..(5)
KHYTA

SEQ ID NO: 90 - protein, 5 aa, Artificial Sequence
  Synthetic Peptide
  D-amino acid (1)..(5)
  Xaa (3)..(3): Xaa = any amino acid
KHXTN

SEQ ID NO: 91 - protein, 5 aa, Artificial Sequence
  Synthetic Peptide
  D-amino acid (1)..(5)
  Xaa (2)..(2): Xaa = any amino acid
KXYTN

SEQ ID NO: 92 - protein, 5 aa, Artificial Sequence
  Synthetic Peptide
  D-amino acid (1)..(5)
  Xaa (5)..(5): Xaa = any amino acid
KHYTX

SEQ ID NO: 93 - protein, 25 aa, Artificial Sequence
  Synthetic Peptide
  Xaa (13)..(13): Xaa = azide-containing amino acid residue with four carbons in the azide-terminated side chain
SQFRVSPLDRTWXLGETVELKSQVL

SEQ ID NO: 94 - protein, 15 aa, Artificial Sequence
  Synthetic Peptide
  Xaa (6)..(6): Xaa = azide-containing amino acid residue with four carbons in the azide-terminated side chain
KCQVLXSNPTSGCSW

SEQ ID NO: 95 - protein, 17 aa, Artificial Sequence
  Synthetic Peptide
  Xaa (8)..(8): Xaa = azide-containing amino acid residue with four carbons in the azide-terminated side chain
RFSGKRLXDTFVLTLSD

SEQ ID NO: 96 - protein, 15 aa, Artificial Sequence
  Synthetic Peptide
  Xaa (10)..(10): Xaa = azide-containing amino acid residue with four carbons in the azide-terminated side chain
QNKPKAAEGXDTQRF

SEQ ID NO: 97 - protein, 25 aa, Artificial Sequence
  Synthetic Peptide
SQFRVSPLDRTWNLGETVELKSQVL

SEQ ID NO: 98 - protein, 17 aa, Artificial Sequence
  Synthetic Peptide
RFSGKRLGDTFVLTLSD

SEQ ID NO: 99 - protein, 15 aa, Artificial Sequence
  Synthetic Peptide
QNKPKAAEGLDTQRF

(19) United States
(12) Patent Application Publication
(10) Pub. No.: US 2020/0291391 A1
(43) Pub. Date: Sep. 17, 2020

(54) CROSS-LINKED EPITOPES AND METHODS OF USE THEREOF

(71) Applicant: Indi Molecular, Inc., Culver City, CA (US)

(72) Inventors: Anders Eliasen, Culver City, CA (US); Bert Tsunyin Lai, Culver City, CA (US)

(21) Appl. No.: 16/817,458

(22) Filed: Mar. 12, 2020

Related U.S. Application Data
(60) Provisional application No. 62/817,020, filed on Mar. 12, 2019.
(60) Provisional application No. 62/933,319, filed on Nov. 8, 2019.

(51) Int. Cl. C12N 15/10; C07K 16/28; G01N 33/68

(52) U.S. Cl. CPC C12N 15/1055; G01N 33/6818; C07K 16/2815

(57) ABSTRACT

Disclosed are peptides and methods useful for identifying and producing capture agents based on epitopes of a target. In particular, disclosed are peptides comprising an epitope, where the peptide is cross-linked, where the epitope corresponds to an epitope of a target, wherein the peptide does not include the entire target, and where the cross-link is a disulfide, preferably a disulfide that naturally occurs in the target or between added or substitute cysteines. Also disclosed are methods of preparing such peptides and methods of using such peptides. In particular, in methods of identifying a target binding compound.

### CROSS REFERENCE TO RELATED APPLICATIONS

This application claims benefit of and priority to U.S. Provisional Application No. 62/817,020, filed Mar. 12, 2019, and U.S. Provisional Application No. 62/933,319, filed Nov. 8, 2019, which are hereby incorporated herein by reference in their entirety.

### FIELD OF THE INVENTION

The disclosed invention is generally in the field of targets for generating capture agents and specifically in the area of cross-linked epitopes for the selection of protein binding agents.

### BACKGROUND OF THE INVENTION

Detection of disease at the earliest stages requires multiplex measurements of key protein biomarkers in biological samples. The availability of high-affinity, highly selective compositions that recognize biomarkers from complex biological mixtures is a critical component for accurate detection of proteins that may indicate disease or changes in health. Peptide affinity agents have been suggested for use as agents for in vitro and/or in vivo detection of disease causing proteins.

Peptide affinity agents that bind to various targets (e.g. proteins) may be identified by screening large peptide libraries, and then using various techniques to identify which peptide library elements exhibit the desired interaction with the target. Those peptide libraries may be biologically synthesized (e.g. bacterial or viral phage display), or they may be chemically synthesized (e.g. one-bead-one-compound (OBOC) libraries). For chemically synthesized libraries, a candidate peptide binder is often first identified using a chemical label. For example, if a protein binds to a particular peptide sequence on a particular bead, then labeling that protein with a fluorescent molecule, or using a similarly labeled antibody to detect the bead-bound protein, can be used to identify the bead that contains the peptide of interest.

Regardless of their preparation method, the sequence of the peptide of interest must then be determined. Typical methods for determining that sequence include mass spectrometric sequencing, or Edman degradation. Thus, peptide libraries for identification of protein affinity agents are preferably readily sequencable by common techniques.

Peptide affinity agents can be targeted to portions of larger protein targets. Use of such protein fragments can be useful to focus hits to target regions and target epitopes of interest. However, target protein fragments isolated from the larger target can be less structured, either forming non-natural structures or presenting multiple less relevant conformations, which can lead to non-productive hits in screens for peptide affinity agents.

Accordingly, there is a need for improved targets for screening for peptide affinity agents. In particular, the is a need for targets that are more structured and more similar to the conformation of the target when in the context of the larger target from which it is derived.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### BRIEF SUMMARY OF THE INVENTION

Disclosed are peptides and methods useful for identifying and producing capture agents based on epitopes of a target. In particular, disclosed are peptides comprising an epitope, where the peptide is cross-linked, where the epitope corresponds to an epitope of a target, wherein the peptide does not include the entire target, and where the cross-link is a disulfide, preferably a disulfide that naturally occurs in the target. The cross-link can also be via added or substitute cysteines or other added or substituted cross-linkable moieties. Also disclosed are methods of preparing a peptide comprising an epitope, where the epitope corresponds to an epitope of a target, where the peptide does not include the entire target, the method comprising cross-linking the peptide, where the cross-link is a disulfide, preferably a disulfide that naturally occurs in the target. The cross-link can also be via added or substitute cysteines or other added or substituted cross-linkable moieties.

In some forms, the target is a target protein. In some forms, the target protein is CD8. In some forms, the epitope comprises amino acids 24 to 33 of CD8. In some forms, the disulfide is between amino acids C22 and C33 of CD8. In some forms, the peptide comprises amino acids 21 to 35 of CD8. In some forms, the epitope comprises amino acids S1 to L25 of CD8 with C22 replaced with S. In some forms, the peptide further comprises an alkyne moiety, an azide moiety, a reporter moiety, or combinations thereof. In some forms, the azide moiety and the alkyne moiety are comprised in an artificial amino acid. In some forms, the artificial amino acid is propargylglycine (Fra). In some forms the artificial amino acid replaces L26 of CD8. In some forms, the reporter moiety is biotin.

In some forms, the peptide further comprises an alkyne moiety, an azide moiety, a reporter moiety, or combinations thereof, where the epitope comprises a phosphorylated amino acid, where the azide moiety and the alkyne moiety of the target peptide are metalorganic molecules that selectively bind to the phospho group on the phosphorylated amino acid. In some forms, the target is a target protein, where the target protein is CD8, where the phosphorylated amino acid is Ser235 or Ser236 of CD8. In some forms, the metalorganic molecule comprises the reporter moiety. In some forms, the reporter moiety is biotin.

Also disclosed are methods for identifying a target binding compound, the method comprising:

(A) contacting a first peptide library with a target peptide comprising an epitope,

where the first peptide library comprises a plurality of first peptide library members, where the first peptide library members optionally individually comprise an alkyne, azide, reporter moiety, or combinations thereof,

where the target peptide is cross-linked, where the epitope corresponds to an epitope of a target, where the target peptide does not include the entire target, where the cross-link is a disulfide, preferably a disulfide that naturally occurs in the target or between added or substitute cysteines, where the target peptide optionally comprises an alkyne moiety, an azide moiety, a reporter moiety, or combinations thereof; and

(B) identifying a first peptide library member with affinity for a first binding site on the epitope.

In some forms, the method can further comprise:

(C) contacting a second peptide library with a composition comprising (i) the target or the target peptide and (ii) the first peptide library member of step B, where, prior to contacting, the first peptide library member of step B is modified to include an alkyne moiety or an azide moiety,

where the second peptide library comprises a plurality of second peptide library members, where the second peptide library members each comprise an azide moiety, an alkyne moiety, or both,

whereby a triazole-linked conjugate is formed between the first peptide library member of step B and a second peptide library member, whereby the triazole-linked second peptide library member is identified as having affinity for a second binding site on the target or the target peptide.

In some forms, the epitope is a distinct molecular surface of the target. In some forms, the target peptide provides a catalytic scaffold for promoting the covalent coupling of the azide moiety and the alkyne moiety to form the triazole linkage. In some forms, the azide moiety and the alkyne moiety of the first peptide library member of step B are comprised in an artificial amino acid. In some forms, the artificial amino acid is propargylglycine (Pra).

In some forms, the epitope comprises a phosphorylated amino acid, where the azide moiety and the alkyne moiety of the target peptide are metalorganic molecules that selectively bind to the phospho group on the phosphorylated amino acid. In some forms, the metalorganic molecule comprises the reporter moiety. In some forms, the metalorganic molecule comprises an azide moiety. In some forms, the reporter moiety is biotin.

In some forms, the method can further comprise:

(D) contacting a third peptide library with a composition comprising (i) the target or the target peptide and (ii) the triazole-linked conjugate formed in step C, wherein, prior to contacting, the triazole-linked conjugate formed in step C is modified to include an alkyne moiety or an azide moiety,

where the third peptide library comprises a plurality of third peptide library members, where the third peptide library members each comprise an azide moiety, an alkyne moiety, or both,

whereby a triazole-linked conjugate is formed between the triazole-linked conjugate formed in step C and a third peptide library member, whereby the triazole-linked third peptide library member is identified as having affinity for a third binding site on the target or the target peptide.

In some forms, the method can further comprise:

(E) contacting a fourth peptide library with a composition comprising (i) the target or the target peptide and (ii) the triazole-linked conjugate formed in step D, where, prior to contacting, the triazole-linked conjugate formed in step D is modified to include an alkyne moiety or an azide moiety,

where the fourth peptide library comprises a plurality of fourth peptide library members, where the fourth peptide library members each comprise an azide moiety, an alkyne moiety, or both,

whereby a triazole-linked conjugate is formed between the triazole-linked conjugate formed in step D and a fourth peptide library member, whereby the triazole-linked fourth peptide library member is identified as having affinity for a fourth binding site on the target or the target peptide.

In some forms, the method can further comprise:

(F) contacting an nth peptide library with a composition comprising (i) the target or the target peptide and (ii) the triazole-linked conjugate formed in the immediately prior contacting step, where, prior to contacting, the triazole-linked conjugate formed in he immediately prior contacting step is modified to include an alkyne moiety or an azide moiety,

where the nth peptide library comprises a plurality of nth peptide library members, where the nth peptide library members each comprise an azide moiety, an alkyne moiety, or both,

whereby a triazole-linked conjugate is formed between the triazole-linked conjugate formed in the immediately prior contacting step and an nth peptide library member, whereby the triazole-linked nth peptide library member is identified as having affinity for an nth binding site on the target or the target peptide.

In some forms, the method can further comprise repeating step F one or more times.

In some forms, the first peptide library member of step B is identified by selecting a first peptide library member linked to the target peptide via a triazole linkage.

In some forms, the second peptide library member of step C is identified by selecting a second peptide library member linked to the first peptide library member via a triazole linkage. In some forms, the third peptide library member of step D is identified by selecting a third peptide library member linked to the triazole-linked conjugate formed in step C via a triazole linkage. In some forms, the fourth peptide library member of step E is identified by selecting a fourth peptide library member linked to the triazole-linked conjugate formed in step D via a triazole linkage. In some forms, the nth peptide library member of step F is identified by selecting an nth peptide library member linked to the triazole-linked conjugate formed in the immediately prior contacting step via a triazole linkage.

In some forms, one or more or the triazole-linked peptide library members are selected by selecting a peptide library member labeled with the reporter moiety.

In some forms, the target is a target protein. In some forms, the method can further comprise testing the first peptide library member of step B for binding to the target protein. In some forms, the method can further comprise testing the triazole-linked conjugate formed in step C for binding to the target protein. In some forms, the method can further comprise testing the triazole-linked conjugate formed in step D for binding to the target protein. In some forms, the method can further comprise testing the triazole-linked conjugate formed in step E for binding to the target protein. In some forms, the method can further comprise testing the triazole-linked conjugate formed in the immediately prior contacting step for binding to the target protein.

In some forms, the second peptide library is contacted with a composition comprising the target peptide and the first peptide library member of step B. In some forms, the second peptide library is contacted with a composition comprising the target and the first peptide library member of step B. In some forms, the third peptide library is contacted with a composition comprising the target peptide and the triazole-linked conjugate formed in step C. In some forms, the third peptide library is contacted with a composition comprising the target and the triazole-linked conjugate formed in step C. In some forms, the fourth peptide library is contacted with a composition comprising the target peptide and the triazole-linked conjugate formed in step D. In some forms, the fourth peptide library is contacted with a composition comprising the target and the triazole-linked conjugate formed in step D. In some forms, the nth peptide library is contacted with a composition comprising the target peptide and the triazole-linked conjugate formed in the immediately prior contacting step. In some forms, the nth peptide library is contacted with a composition comprising the target and the triazole-linked conjugate formed in the immediately prior contacting step.

In some forms, the epitope is a distinct molecular surface of the target.

In some forms, the method can further comprise determining the peptide sequence of the first peptide library member of step B. In some forms, the method can further comprise determining the peptide sequence of the second peptide library member of step C. In some forms, the method can further comprise determining the peptide sequence of the third peptide library member of step D. In some forms, the method can further comprise determining the peptide sequence of the fourth peptide library member of step E. In some forms, the method can further comprise determining the peptide sequence of the nth peptide library member of step F. In some forms, the peptide sequence of one or more of the peptide library members is determined by Edman degradation.

In some forms, the method can further comprise modifying the triazole linked conjugate formed in step C to contain a triazole or alkyne and contacting the modified conjugate with the target or the target peptide and a third peptide library, the third peptide library comprising a plurality of third peptide library members, each third peptide library member comprising an azide or alkyne, thereby forming a triazole linkage between the modified conjugate and a member of the third peptide library, wherein the third peptide library member is identified as having affinity for a third binding site on the target or the target peptide.

In some forms, the method can further comprise modifying the triazole linked conjugate formed between the modified conjugate and the member of the third peptide library to contain a triazole or alkyne and contacting the modified conjugate with the target or the target peptide and a fourth peptide library, the fourth peptide library comprising a plurality of fourth peptide library members, each fourth peptide library member comprising an azide or alkyne, thereby forming a triazole linkage between the modified conjugate and a member of the fourth peptide library, wherein the fourth peptide library member is identified as having affinity for a fourth binding site on the target or the target peptide.

In some forms, the method can further comprise modifying the triazole linked conjugate formed between the modified conjugate and the identified fourth peptide library member to contain a triazole or alkyne and contacting the modified conjugate with the target or the target peptide and an nth peptide library, the nth peptide library comprising a plurality of nth peptide library members, each nth peptide library member comprising an azide or alkyne, thereby forming a triazole linkage between the modified conjugate and a member of the nth peptide library, wherein the nth peptide library member is identified as having affinity for an nth binding site on the target or the target peptide.

In some forms, the method can further comprise:

(i) modifying the triazole linked conjugate formed between the modified conjugate and the identified nth peptide library member to contain a triazole or alkyne and

(ii) contacting the modified conjugate with the target or the target peptide and an N+1th peptide library, the N+1th peptide library comprising a plurality of N+1th peptide library members, each n+1th peptide library member comprising an azide or alkyne, thereby forming a triazole linkage between the modified conjugate and a member of the N+1th peptide library, the n+1th peptide library member having affinity for an n+1th binding site on the target or the target peptide.

In some forms, the method can further comprise repeating steps (i) and (ii) one or more times.

In some forms, the first binding site is an epitope. In some forms, the second binding site is a second epitope. In some forms, the third binding site is a third epitope. In some forms, the fourth binding site is a fourth epitope. In some forms, the target is a protein. In some forms, the protein is an enzyme or cell surface protein. In some forms, the protein is CD8.

In some forms, the target peptide comprises a linkage to a reporter moiety, wherein the reporter moiety comprises polyethylene glycol (PEG), biotin, thiol, a fluorophore, or combinations thereof. In some forms, the fluorophore is carboxyfluorescein (FAM), fluorescein isothiocyanate (FITC), Cyanine-5 (Cy5), tetramethylrhodamine (TRITC) or Carboxytetramethylrhodamine (TAMRA).

Also disclosed are ligands and multiligands (for example, biligands, triligands, quadligands, etc.) identified and/or produced by the disclosed methods.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIG. 1 is a diagram of a CD8 homodimer interacting with an MHC domain. Epitope B2 is shown as the white space filling structure. The equilibrated CD8 homodimer is shown as a grey space filling structure. The equilibrated MHC domain is shown as a wire structure.

FIG. 2 is a schematic providing an illustration of one form of epitope targeting.

FIGS. 3A-3D are schematics providing an illustration of one form of screening for molecules that bind to the targeted epitope. FIG. 3A depicts incubation of the polypeptide fragment containing the epitope and the substituted or altered amino acid with a large molecular library. FIG. 3B shows the incubation step. FIG. 3C shows use of the label on the polypeptide fragment to generate a signal that discriminates those elements of the molecular library that are not. FIG. 3D shows separation of molecular library elements that are covalently coupled to the peptide from those that are not. FIGS. 4A-4D are schematics of epitopes (light structure) on a CD8 homodimer (grey structure). FIG. 4A illustrates epitope A, covering S1 to R10 of CD8 (SQFRVSPLDR; SEQ ID NO:2). This epitope interacts with the MHC domain. FIG. 4B illustrates epitope B, covering V24 to C33 of CD8 (VLLSNPTSGC; SEQ ID NO:3). This epitope interacts with the MHC domain. FIG. 4C illustrates epitopes F (F1/F2), covering F37 to L49 of CD8 (FQPRGAAASPTFL; SEQ ID NO:7). This epitope is located adjacent to the interface of the CD8a homodimer, forming a cavity with sufficient volume to accommodate a PCC. FIG. 4D illustrates epitope G, covering L50 to A60 of CD8 (LYLSQNKPKAA; SEQ ID NO:8). This epitope interacts with the MHC domain. Epitopes F (F1/F2) can be found adjacent to the interface of the CD8a homodimer. Epitopes A, B, and G interacts with the MHC. The desired screening order: Epitope G, Epitope F (F1/F2), Epitope A, Epitope B. All of these epitopes provide a large solvent accessible surface, which makes for preferred epitopes.

FIG. 5 is a ribbon diagram of epitopes B2 and E on a CD8 homodimer. The B2 epitope is (a) the top half of the third from the back of the four B-sheet ribbons on the left, (b) the top half of the second from the back of the four B-sheet ribbons to the right of the leftmost four B-sheet ribbons on the left, and (c) the unstructured loop connecting the two B-sheet portions. The E epitope is (a) the top half of the third from the back of the four B-sheet ribbons to the right of the leftmost four B-sheet ribbons, (b) the front B-sheet ribbon (which is interrupted by an unstructured section) of the four B-sheet ribbons to the right of the leftmost four B-sheet ribbons, and (c) the unstructured loop connecting the two B-sheet portions.

FIGS. 6A and 6B are graphs of binding of hits from a constrained epitope B2 screen to CD8a or non-specific binding to PSMA by ELISA using three different PCC concentrations (4000 nM, 400 nM, 10 nM).

FIG. 7 is a diagram of khytn ligand (SEQ ID NO:82).

FIG. 8 is a diagram of the alkyne-containing tfpkk peptide (SEQ ID NO:21) coupled to a bead.

FIG. 9 is a diagram of azide- and biotin-containing epitopes.

FIG. 10 is a diagram of tfpkk ligand (SEQ ID NO:21).

FIG. 11 is a graph of flow cytometry binding of tfpkk (SEQ ID NO:21), khytn (SEQ ID NO:82), nsprw (SEQ ID NO:83), and herlk (SEQ ID NO:84) ligands to SupT1 (CD8+) cells and Jurkat (CD8-) cells.

FIG. 12 is a graph of flow cytometry binding of ahytn (SEQ ID NO:85), kaytn (SEQ ID NO:86), khatn (SEQ ID NO:87), khyan (SEQ ID NO:88), khyta (SEQ ID NO:89), and khytn (SEQ ID NO:82) to SupT1 (CD8+) cells and Jurkat (CD8-) cells.

FIG. 13 is a diagram of screening a peptide library with a cocktail of four CD8a epitopes to discover CD8 ligands.

### DETAILED DESCRIPTION OF THE INVENTION

The disclosed method and compositions can be understood more readily by reference to the following detailed description of particular forms and the Example included therein and to the Figures and their previous and following description.

In screening for peptide affinity agents, such as capture agents and protein-catalyzed capture (PCC) agents, it was observed that hits that result from epitope screens (e.g., epitope targeting) may not bind efficiently to the full-length target (e.g., target protein). It was realized that the relatively short epitope peptides used as target peptides may not adopt topology or tertiary structures (i.e., conformation) that mimic the full-length target. It was realized that constructing synthetic epitopes (as target peptides) that adopt secondary or tertiary structures that mimic (or more closely mimic) their conformation in the full-length target could improve the fraction and quality of hits in screening for agents that bind that epitope and the full-length target.

By producing such synthetic epitopes it was discovered that screens using the synthetic epitopes identified hits more efficiently and provided high affinity and high specificity hits. In particular, a contiguous sequence present in CD8, which incorporates a naturally occurring disulfide was identified. A synthetic epitope based on this CD8 sequence was produced in which the disulfide is formed in the synthetic epitope as a cross-link. This cross-linked synthetic epitope more closely resembles the solvent exposed surface of the CD8 protein. Synthetic epitopes based on PSMA sequences in which cysteines have been introduced produce epitopes cross-linked via the cysteines.

Disclosed are peptides and methods useful for identifying and producing capture agents based on epitopes of a target. In particular, disclosed are peptides comprising an epitope, where the peptide is cross-linked, where the epitope corresponds to an epitope of a target, wherein the peptide does not include the entire target, and where the cross-link is a disulfide, preferably a disulfide that naturally occurs in the target or between added or substitute cysteines. Also disclosed are methods of preparing a peptide comprising an epitope, where the epitope corresponds to an epitope of a target, where the peptide does not include the entire target, the method comprising cross-linking the peptide, where the cross-link is a disulfide, preferably a disulfide that naturally occurs in the target or between added or substitute cysteines.

In some forms, the target is a target protein. In some forms, the target protein is CD8. In some forms, the epitope comprises amino acids 24 to 33 of CD8. In some forms, the disulfide is between amino acids C22 and C33 of CD8. In some forms, the peptide comprises amino acids 21 to 35 of CD8. In some forms, the epitope comprises amino acids S1 to L25 of CD8 with C22 replaced with S. In some forms, the peptide further comprises an alkyne moiety, an azide moiety, a reporter moiety, or combinations thereof. In some forms, the azide moiety and the alkyne moiety are comprised in an artificial amino acid. In some forms, the artificial amino acid is propargylglycine (Fra). In some forms the artificial amino acid replaces L26 of CD8. In some forms, the reporter moiety is biotin.

In some forms, the peptide further comprises an alkyne moiety, an azide moiety, a reporter moiety, or combinations thereof, where the epitope comprises a phosphorylated amino acid, where the azide moiety and the alkyne moiety of the target peptide are metalorganic molecules that selectively bind to the phospho group on the phosphorylated amino acid. In some forms, the target is a target protein, where the target protein is CD8, where the phosphorylated amino acid is Ser235 or Ser236 of CD8. In some forms, the metalorganic molecule comprises the reporter moiety. In some forms, the reporter moiety is biotin.

Also disclosed are methods for identifying a target binding compound, the method comprising:

(A) contacting a first peptide library with a target peptide comprising an epitope,

where the first peptide library comprises a plurality of first peptide library members, where the first peptide library members optionally individually comprise an alkyne, azide, reporter moiety, or combinations thereof,

where the target peptide is cross-linked, where the epitope corresponds to an epitope of a target, where the target peptide does not include the entire target, where the cross-link is a disulfide, preferably a disulfide that naturally occurs in the target or between added or substitute cysteines, where the target peptide optionally comprises an alkyne moiety, an azide moiety, a reporter moiety, or combinations thereof; and

(B) identifying a first peptide library member with affinity for a first binding site on the epitope.

In some forms, the method can further comprise:

(C) contacting a second peptide library with a composition comprising (i) the target or the target peptide and (ii) the first peptide library member of step B, where, prior to contacting, the first peptide library member of step B is modified to include an alkyne moiety or an azide moiety,

where the second peptide library comprises a plurality of second peptide library members, where the second peptide library members each comprise an azide moiety, an alkyne moiety, or both,

whereby a triazole-linked conjugate is formed between the first peptide library member of step B and a second peptide library member, whereby the triazole-linked second peptide library member is identified as having affinity for a second binding site on the target or the target peptide.

In some forms, the epitope is a distinct molecular surface of the target. In some forms, the target peptide provides a catalytic scaffold for promoting the covalent coupling of the azide moiety and the alkyne moiety to form the triazole linkage. In some forms, the azide moiety and the alkyne moiety of the first peptide library member of step B are comprised in an artificial amino acid. In some forms, the artificial amino acid is propargylglycine (Fra).

In some forms, the epitope comprises a phosphorylated amino acid, where the azide moiety and the alkyne moiety of the target peptide are metalorganic molecules that selectively bind to the phospho group on the phosphorylated amino acid. In some forms, the metalorganic molecule comprises the reporter moiety. In some forms, the metalorganic molecule comprises an azide moiety. In some forms, the reporter moiety is biotin.

In some forms, the method can further comprise:

(D) contacting a third peptide library with a composition comprising (i) the target or the target peptide and (ii) the triazole-linked conjugate formed in step C, wherein, prior to contacting, the triazole-linked conjugate formed in step C is modified to include an alkyne moiety or an azide moiety,

where the third peptide library comprises a plurality of third peptide library members, where the third peptide library members each comprise an azide moiety, an alkyne moiety, or both,

whereby a triazole-linked conjugate is formed between the triazole-linked conjugate formed in step C and a third peptide library member, whereby the triazole-linked third peptide library member is identified as having affinity for a third binding site on the target or the target peptide.

In some forms, the method can further comprise:

(E) contacting a fourth peptide library with a composition comprising (i) the target or the target peptide and (ii) the triazole-linked conjugate formed in step D, where, prior to contacting, the triazole-linked conjugate formed in step D is modified to include an alkyne moiety or an azide moiety,

where the fourth peptide library comprises a plurality of fourth peptide library members, where the fourth peptide library members each comprise an azide moiety, an alkyne moiety, or both,

whereby a triazole-linked conjugate is formed between the triazole-linked conjugate formed in step D and a fourth peptide library member, whereby the triazole-linked fourth peptide library member is identified as having affinity for a fourth binding site on the target or the target peptide.

In some forms, the method can further comprise:

(F) contacting an nth peptide library with a composition comprising (i) the target or the target peptide and (ii) the triazole-linked conjugate formed in the immediately prior contacting step, where, prior to contacting, the triazole-linked conjugate formed in he immediately prior contacting step is modified to include an alkyne moiety or an azide moiety,

where the nth peptide library comprises a plurality of nth peptide library members, where the nth peptide library members each comprise an azide moiety, an alkyne moiety, or both,

whereby a triazole-linked conjugate is formed between the triazole-linked conjugate formed in the immediately prior contacting step and an nth peptide library member, whereby the triazole-linked nth peptide library member is identified as having affinity for an nth binding site on the target or the target peptide.

In some forms, the method can further comprise repeating step F one or more times.

In some forms, the first peptide library member of step B is identified by selecting a first peptide library member linked to the target peptide via a triazole linkage. In some forms, the second peptide library member of step C is identified by selecting a second peptide library member linked to the first peptide library member via a triazole linkage. In some forms, the third peptide library member of step D is identified by selecting a third peptide library member linked to the triazole-linked conjugate formed in step C via a triazole linkage. In some forms, the fourth peptide library member of step E is identified by selecting a fourth peptide library member linked to the triazole-linked conjugate formed in step D via a triazole linkage. In some forms, the nth peptide library member of step F is identified by selecting an nth peptide library member linked to the triazole-linked conjugate formed in the immediately prior contacting step via a triazole linkage.

In some forms, one or more or the triazole-linked peptide library members are selected by selecting a peptide library member labeled with the reporter moiety.

In some forms, the target is a target protein. In some forms, the method can further comprise testing the first peptide library member of step B for binding to the target protein. In some forms, the method can further comprise testing the triazole-linked conjugate formed in step C for binding to the target protein. In some forms, the method can further comprise testing the triazole-linked conjugate formed in step D for binding to the target protein. In some forms, the method can further comprise testing the triazole-linked conjugate formed in step E for binding to the target protein. In some forms, the method can further comprise testing the triazole-linked conjugate formed in the immediately prior contacting step for binding to the target protein.

In some forms, the second peptide library is contacted with a composition comprising the target peptide and the first peptide library member of step B. In some forms, the second peptide library is contacted with a composition comprising the target and the first peptide library member of step B. In some forms, the third peptide library is contacted with a composition comprising the target peptide and the triazole-linked conjugate formed in step C. In some forms, the third peptide library is contacted with a composition comprising the target and the triazole-linked conjugate formed in step C. In some forms, the fourth peptide library is contacted with a composition comprising the target peptide and the triazole-linked conjugate formed in step D. In some forms, the fourth peptide library is contacted with a composition comprising the target and the triazole-linked conjugate formed in step D. In some forms, the nth peptide library is contacted with a composition comprising the target peptide and the triazole-linked conjugate formed in the immediately prior contacting step. In some forms, the nth peptide library is contacted with a composition comprising the target and the triazole-linked conjugate formed in the immediately prior contacting step.

In some forms, the epitope is a distinct molecular surface of the target.

In some forms, the method can further comprise determining the peptide sequence of the first peptide library member of step B. In some forms, the method can further comprise determining the peptide sequence of the second peptide library member of step C. In some forms, the method can further comprise determining the peptide sequence of the third peptide library member of step D. In some forms, the method can further comprise determining the peptide sequence of the fourth peptide library member of step E. In some forms, the method can further comprise determining the peptide sequence of the nth peptide library member of step F. In some forms, the peptide sequence of one or more of the peptide library members is determined by Edman degradation.

In some forms, the method can further comprise modifying the triazole linked conjugate formed in step C to contain a triazole or alkyne and contacting the modified conjugate with the target or the target peptide and a third peptide library, the third peptide library comprising a plurality of third peptide library members, each third peptide library member comprising an azide or alkyne, thereby forming a triazole linkage between the modified conjugate and a member of the third peptide library, wherein the third peptide library member is identified as having affinity for a third binding site on the target or the target peptide.

In some forms, the method can further comprise modifying the triazole linked conjugate formed between the modified conjugate and the member of the third peptide library to contain a triazole or alkyne and contacting the modified conjugate with the target or the target peptide and a fourth peptide library, the fourth peptide library comprising a plurality of fourth peptide library members, each fourth peptide library member comprising an azide or alkyne, thereby forming a triazole linkage between the modified conjugate and a member of the fourth peptide library, wherein the fourth peptide library member is identified as having affinity for a fourth binding site on the target or the target peptide.

In some forms, the method can further comprise modifying the triazole linked conjugate formed between the modified conjugate and the identified fourth peptide library member to contain a triazole or alkyne and contacting the modified conjugate with the target or the target peptide and an nth peptide library, the nth peptide library comprising a plurality of nth peptide library members, each nth peptide library member comprising an azide or alkyne, thereby forming a triazole linkage between the modified conjugate and a member of the nth peptide library, wherein the nth peptide library member is identified as having affinity for an nth binding site on the target or the target peptide.

In some forms, the method can further comprise:

(i) modifying the triazole linked conjugate formed between the modified conjugate and the identified nth peptide library member to contain a triazole or alkyne and

(ii) contacting the modified conjugate with the target or the target peptide and an N+1th peptide library, the N+1th peptide library comprising a plurality of N+1th peptide library members, each N+1th peptide library member comprising an azide or alkyne, thereby forming a triazole linkage between the modified conjugate and a member of the N+1th peptide library, the N+1th peptide library member having affinity for an N+1th binding site on the target or the target peptide.

In some forms, the method can further comprise repeating steps (i) and (ii) one or more times.

In some forms, the first binding site is an epitope. In some forms, the second binding site is a second epitope. In some forms, the third binding site is a third epitope.

In some forms, the fourth binding site is a fourth epitope. In some forms, the target is a protein. In some forms, the protein is an enzyme or cell surface protein. In some forms, the protein is CD8.

In some forms, the target peptide comprises a linkage to a reporter moiety, wherein the reporter moiety comprises polyethylene glycol (PEG), biotin, thiol, a fluorophore, or combinations thereof. In some forms, the fluorophore is carboxyfluorescein (FAM), fluorescein isothiocyanate (FITC), Cyanine-5 (Cy5), tetramethylrhodamine (TRITC) or Carboxytetramethylrhodamine (TAMRA).

Also disclosed are ligands and multiligands (for example, biligands, triligands, quadligands, etc.) identified and/or produced by the disclosed methods. Examples of such ligands include hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), and teGwf (SEQ ID NO:20). Such ligands can be used together, such as in multiligands, with other ligands that have not been identified and/or produced by the disclosed methods. Examples of such ligands include krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID

NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), and hGrGh (SEQ ID NO:74).

Examples of preferred ligands include tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), khyta (SEQ ID NO:89), nsprw (SEQ ID NO:83), and herlk (SEQ ID NO:84).

Linkers between ligands in multiligands can be chosen based on the distance between the epitopes recognized by the ligands, by screening for target catalyzed coupling of ligands in a mix with linkers of different linkers, or a combination.

Also disclosed are ligands and multiligands identified and/or produced by the disclosed methods. For example, disclosed are ligands identified and/or produced by:

(A) contacting a first peptide library with a target peptide comprising an epitope,

where the first peptide library comprises a plurality of first peptide library members, where the first peptide library members optionally individually comprise an alkyne, azide, reporter moiety, or combinations thereof,

where the target peptide is cross-linked, where the epitope corresponds to an epitope of a target, where the target peptide does not include the entire target, where the cross-link is a disulfide, preferably a disulfide that naturally occurs in the target or between added or substitute cysteines, where the target peptide optionally comprises an alkyne moiety, an azide moiety, a reporter moiety, or combinations thereof; and

(B) identifying a first peptide library member with affinity for a first binding site on the epitope, where the identified first peptide library member is the ligand identified and/or produced.

In some forms, other ligands and multiligands can be identified and/or produced by:

(C) contacting a second peptide library with a composition comprising (i) the target or the target peptide and (ii) the first peptide library member of step B, where, prior to contacting, the first peptide library member of step B is modified to include an alkyne moiety or an azide moiety,

where the second peptide library comprises a plurality of second peptide library members, where the second peptide library members each comprise an azide moiety, an alkyne moiety, or both,

whereby a triazole-linked conjugate is formed between the first peptide library member of step B and a second peptide library member, whereby the triazole-linked second peptide library member is identified as having affinity for a second binding site on the target or the target peptide, where the identified second peptide library member is another ligand identified and/or produced, where the triazole-linked conjugate is a biligand identified and/or produced.

In some forms, the epitope is a distinct molecular surface of the target. In some forms, the target peptide provides a catalytic scaffold for promoting the covalent coupling of the azide moiety and the alkyne moiety to form the triazole linkage. In some forms, the azide moiety and the alkyne moiety of the first peptide library member of step B are comprised in an artificial amino acid. In some forms, the artificial amino acid is propargylglycine (Fra).

In some forms, the epitope comprises a phosphorylated amino acid, where the azide moiety and the alkyne moiety of the target peptide are metalorganic molecules that selectively bind to the phospho group on the phosphorylated amino acid. In some forms, the metalorganic molecule comprises the reporter moiety. In some forms, the metalorganic molecule comprises an azide moiety. In some forms, the reporter moiety is biotin.

In some forms, other ligands and multiligands can be identified and/or produced by:

(D) contacting a third peptide library with a composition comprising (i) the target or the target peptide and (ii) the triazole-linked conjugate formed in step C, wherein, prior to contacting, the triazole-linked conjugate formed in step C is modified to include an alkyne moiety or an azide moiety,

where the third peptide library comprises a plurality of third peptide library members, where the third peptide library members each comprise an azide moiety, an alkyne moiety, or both,

whereby a triazole-linked conjugate is formed between the triazole-linked conjugate formed in step C and a third peptide library member, whereby the triazole-linked third peptide library member is identified as having affinity for a third binding site on the target or the target peptide, where the identified third peptide library member is another ligand identified and/or produced, where the triazole-linked conjugate is a biligand identified and/or produced.

In some forms, other ligands and multiligands can be identified and/or produced by:

(E) contacting a fourth peptide library with a composition comprising (i) the target or the target peptide and (ii) the triazole-linked conjugate formed in step D, where, prior to contacting, the triazole-linked conjugate formed in step D is modified to include an alkyne moiety or an azide moiety,

where the fourth peptide library comprises a plurality of fourth peptide library members, where the fourth peptide library members each comprise an azide moiety, an alkyne moiety, or both,

whereby a triazole-linked conjugate is formed between the triazole-linked conjugate formed in step D and a fourth peptide library member, whereby the triazole-linked fourth peptide library member is identified as having affinity for a fourth binding site on the target or the target peptide, where the identified fourth peptide library member is another ligand identified and/or produced, where the triazole-linked conjugate is a biligand identified and/or produced.

In some forms, other ligands and multiligands can be identified and/or produced by:

(F) contacting an nth peptide library with a composition comprising (i) the target or the target peptide and (ii) the triazole-linked conjugate formed in the immediately prior contacting step, where, prior to contacting, the triazole-linked conjugate formed in he immediately prior contacting step is modified to include an alkyne moiety or an azide moiety,

where the nth peptide library comprises a plurality of nth peptide library members, where the nth peptide library members each comprise an azide moiety, an alkyne moiety, or both,

whereby a triazole-linked conjugate is formed between the triazole-linked conjugate formed in the immediately prior contacting step and an nth peptide library member, whereby the triazole-linked nth peptide library member is identified as having affinity for an nth binding site on the target or the target peptide, where the identified nth peptide library member is another ligand identified and/or produced, where the triazole-linked conjugate is a biligand identified and/or produced.

In some forms, other ligands and multiligands can be identified and/or produced by repeating step F one or more times, where the identified nth peptide library member is another ligand identified and/or produced, where the triazole-linked conjugate is a biligand identified and/or produced.

The term “epitope” as used herein refers to a distinct molecular surface of a target protein capable of catalyzing the assembly of a PCC from a library of molecular building blocks. Typically, the epitope is a polypeptide and it can act on its own as a finite sequence of 20-40 amino acids.

The term “epitope targeting” as used herein refers to a process by which an anchor ligand is selected by an epitope-catalyzed process where a synthetic polypeptide epitope presenting a first functional group interacts with a library of possible anchor ligands presenting a second functional group to result in the formation of a covalent linkage between the polypeptide and anchor ligand. The selected anchor ligand displays affinity toward both the polypeptide epitope and the full-length (native) target protein. The polypeptide epitope dictates the sequence and binding site of the anchor ligand, and ultimately the capture agent or PCC.

The same epitope, now existing as part of the larger protein, can be involved in catalyzing the assembly of a PCC biligand from the previously selected anchor ligand (modified with a second functional group) and a library of molecular building blocks (modified with a first functional group) in a protein-catalyzed process. This protein-catalyzed process can then repeated to assemble a PCC triligand from the previously selected biligand (modified with a third functional group) and a library of molecular building blocks (modified with a fourth functional group).

Large biomolecules, such as proteins, can be characterized by a diverse landscape of chemical properties that vary significantly across different parts of the molecule. Specific regions of a biomolecule surface are referred to as epitopes. It is often desirable to develop molecules that bind specifically to one epitope on a protein, but not to other epitopes on that protein, or to other proteins. Monoclonal antibodies, which are biological products, are developed to bind to specific epitopes on specific proteins. However, there is not a good way, using chemical synthesis approaches, to target a particular epitope on a protein, unless that epitope also happens to fit very special criteria—i.e. the epitope contains a small molecule binding pocket, and so provides a unique energy well for attracting small molecule binders, relative to the rest of the protein. The vast majority of protein epitopes do not fit these special criteria. Described is an approach that can guide the development of highly specific molecular binders to general classes of protein epitopes.

An approach for synthesizing molecules that bind to specific parts (epitopes) of large protein biomolecules is described and demonstrated. The disclosed method includes first preparing a peptide or polypeptide fragment of a specific protein. That polypeptide can be site-specifically modified near the region of the epitope of interest, by either substituting one of the naturally occurring amino acids for an artificial amino acid, or the polypeptide fragment is modified after synthesis by chemically altering a specific amino acid. In both cases, the modification results in the presentation of either an acetylene or an azide chemical group near the site-specific modification. That azide (acetylene) containing fragment is then incubated with a very large molecular library. This library, while typically chemically diverse, is also characterized by the fact that each element contains an acetylene (or, instead, each element contains an azide) group. The incubation can be done under conditions that the modified polypeptide fragment can provide a catalytic scaffold for promoting the covalent coupling between select library elements and the polypeptide fragment. In some forms, it promotes this coupling by catalyzing the formation of a triazole linkage that is the reaction product of the acetylene and azide groups. In some forms, the selectivity of this catalyzed process is very high. This means that only a very small fraction of the elements in the molecular library will be coupled. Those elements are identified through analytical techniques, and then tested for binding to the polypeptide fragment, or to the entire protein biomolecule from which the polypeptide fragment was extracted. This approach provides a route towards identifying molecules that selectively bind to the intended epitope of the protein target. Approaches known in the art can then be utilized to increase the selectivity and the affinity of the identified binders, without sacrificing their epitope selective binding characteristics.

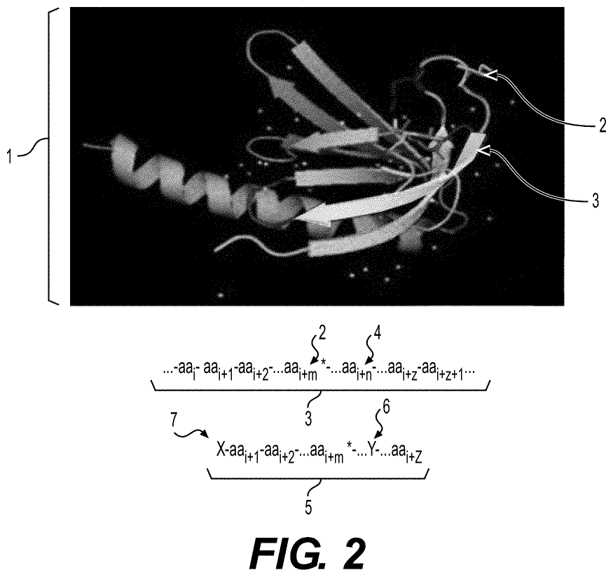

FIG. 2 provides an illustration of one form of the epitope targeting process. A protein target (1) is selected. The protein target (1) has a specific epitope (2) that is of interest for developing capture agent molecule that will bind to that location. That epitope can be a specific amino acid residue (2) associated with a particular peptide or polypeptide fragment (3) of the entire protein (1), or it can be a larger region of the protein (1) containing several amino acids. The epitope is located within a region of the protein that is characterized by a known sequence of amino acids (3). An amino acid near (or within) the epitope (4) is identified for either substitution with an artificial amino acid, or some other specific chemical modification to introduce an azide or acetylene group onto that site. A polypeptide fragment (5) of the protein that contains the targeted epitope is synthesized, but with two modifications. First, (4) is either substituted or chemically modified so as to provide an azide or acetylene group. Second, a site on the polypeptide is modified (7) with a label (a fluorophore or biotin group, for example) for use during the screening steps. There are many ways through which this label can be introduced.

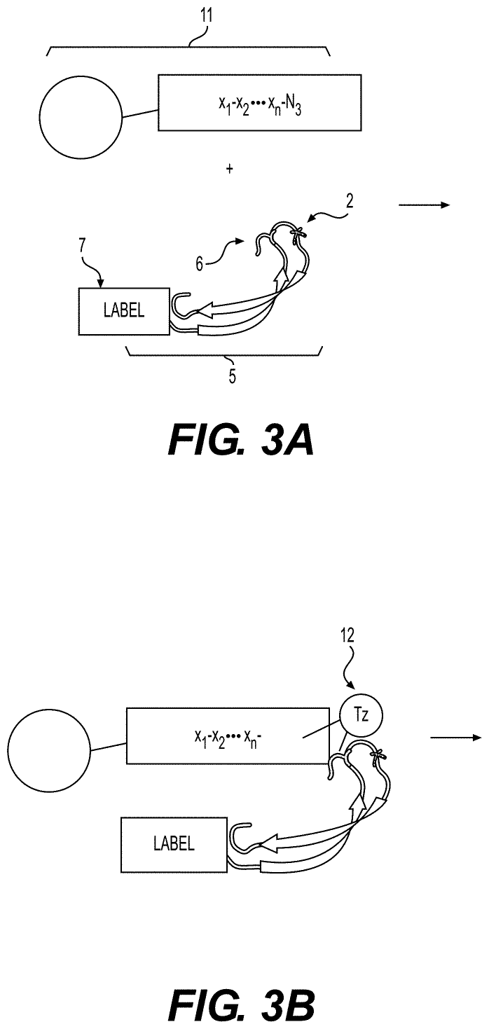

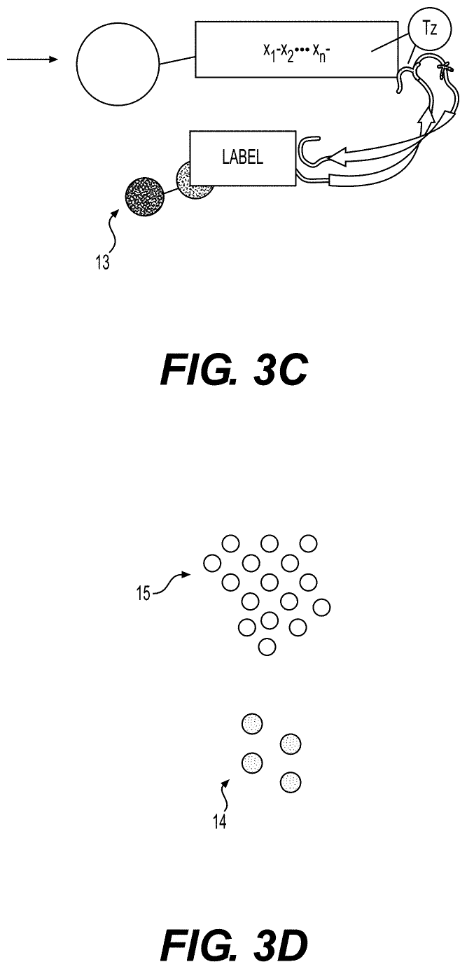

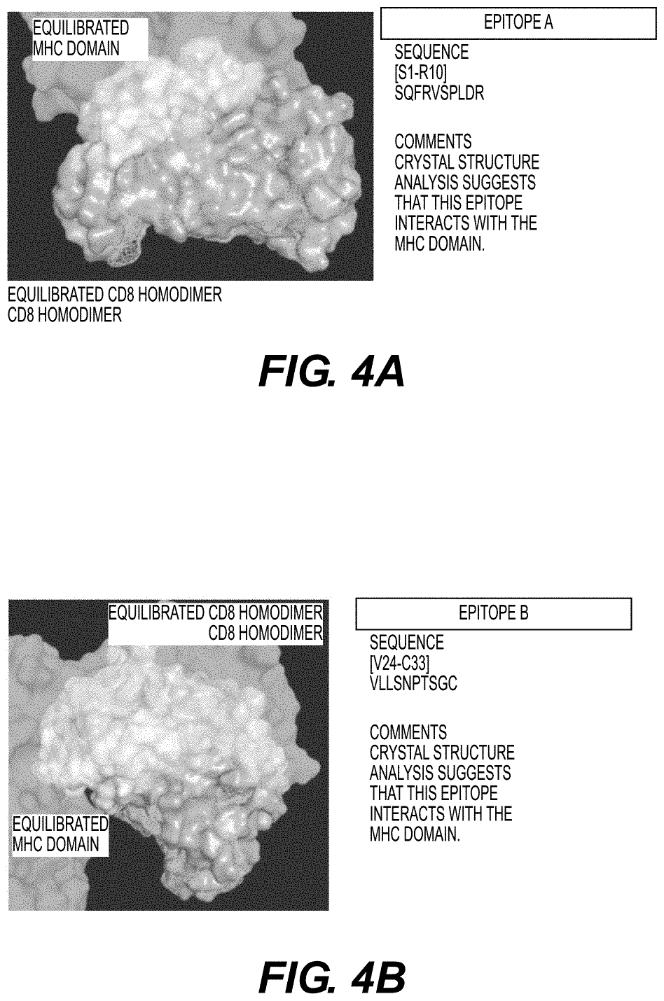

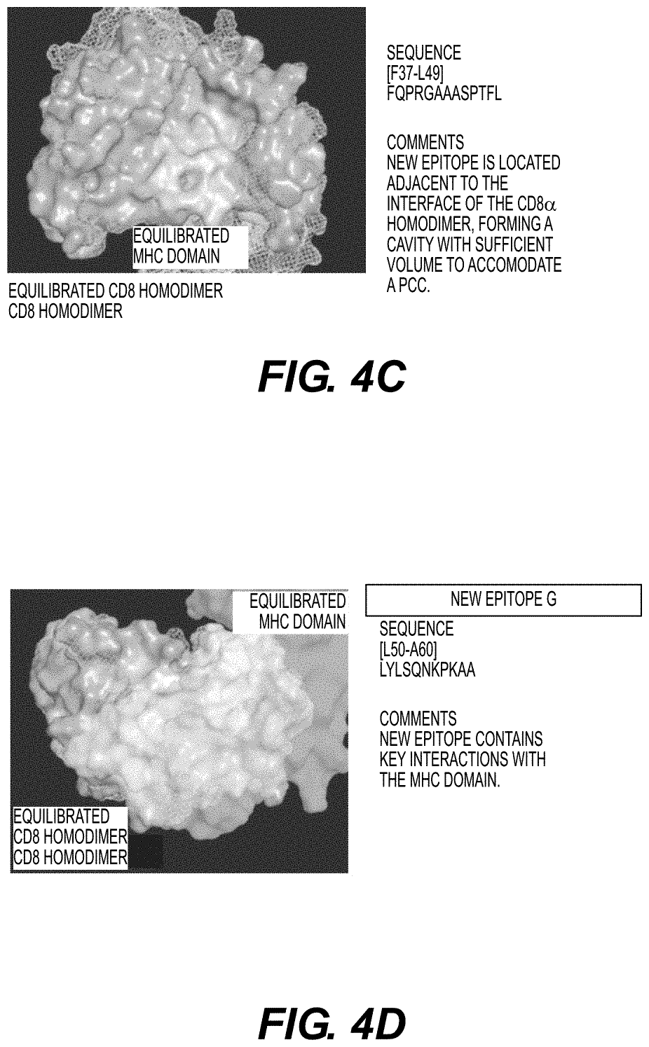

FIG. 3 provides an illustration of one form of screening for molecules that bind to the targeted epitope. FIG. 3A shows the polypeptide fragment (5) containing the epitope (2), the substituted or altered amino acid (6), and the label (7) being incubated with a large molecular library (11). In this instance, the library is shown presenting an azide group, which would imply that the polypeptide fragment would present an acetylene group at (6). In this instance, the azide group is at the n-terminus of the molecule, but this is not a requirement. In this instance, the molecular library is also represented as a bead-based library, but this is also not a requirement. FIG. 3B shows that during the incubation step, the polypeptide fragment provides a catalytic scaffold for promoting the covalent coupling of the azide and acetylene groups to form a triazole linkage (12), so that the polypeptide fragment is now covalently bonded to very specific elements of the molecular library. At this point, the molecular library is cleared of all free polypeptide via standard washing steps. FIG. 3C shows that the label on the polypeptide fragment can be utilized to generate a signal (13) that discriminates those elements of the molecular library that are covalently coupled to the polypeptide fragment, from those library elements that are not. FIG. 3D shows that the molecular library elements that are covalently coupled to the peptide (14) can be separated from those library elements that are not (15), and subjected to analysis to identify which molecules are potential binders.

The result of the steps described in FIGS. 2 and 3 is the identification of a small number of molecules that potentially are selective binders to the epitope of interest. These are referred to herein as “hits.” Those hits, or a representative set of those hits, can then tested in standard biological assays, such as immunoprecipitation assays, for binding to the protein target of interest. If no binders are identified, then there are several options, which can be tested separately, or in combination. Those options include the following. The process described in FIG. 3 can be repeated, but with a higher concentration of the modified polypeptide fragment (5) present during the incubation step. The process described in FIG. 3 can be repeated, but using a larger (more chemically diverse) molecular library (11). The polypeptide fragment (5) can be modified in a different way in preparation for the screen, and then the steps of FIG. 3 repeated.

The term “capture agent” as used herein refers to a protein-catalyzed capture (PCC) agent that comprises one or more target-binding moieties and which specifically binds to a target, such as a target protein, via those target-binding moieties. Each target-binding moiety exhibits binding affinity for the target, either individually or in combination with other target-binding moieties. In some forms, each target-binding moiety binds to the target, via one or more non-covalent interactions, including for example hydrogen bonds, hydrophobic interactions, and van der Waals interactions. A capture agent can comprise one or more organic molecules, including for example polypeptides, peptides, polynucleotides, and other non-polymeric molecules.

The terms “polypeptide,” “peptide,” and “protein” are used interchangeably herein to refer to an amino acid sequence comprising a polymer of amino acid residues. The terms apply to amino acid polymers in which one or more amino acid residues is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids, and isomers thereof. Naturally occurring amino acids are those encoded by the genetic code, as well as those amino acids that are later modified, e.g., hydroxyproline, carboxyglutamate, 0-phosphoserine, and isomers thereof. The term “amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” refers to chemical compounds that have a structure that is different from the general chemical structure of an amino acid, but that functions in a manner similar to a naturally occurring amino acid Amino acids can be referred to herein by either their commonly known three letter symbols or by the one-letter symbols recommended by the IUPAC-IUB Biochemical Nomenclature Commission.

The term “non-natural amino acid” as used herein refers to an amino acid that is different from the twenty naturally occurring amino acids (alanine, arginine, glycine, asparagine, aspartic acid, cysteine, glutamine, glutamic acid, serine, threonine, histidine, lysine, methionine, proline, valine, isoleucine, leucine, tyrosine, tryptophan, phenylalanine) in its side chain functionality. The non-natural amino acid can be a close analog of one of the twenty natural amino acids, or it can introduce a completely new functionality and chemistry, as long as the hydrophobicity of the non-natural amino acid is either equivalent to or greater than that of the natural amino acid. The non-natural amino acid can either replace an existing amino acid in a protein (substitution), or be an addition to the wild type sequence (insertion). The incorporation of non-natural amino acids can be accomplished by known chemical methods including solid-phase peptide synthesis or native chemical ligation, or by biological methods.

The terms “specific binding,” “selective binding,” “selectively binds,” or “specifically binds” as used herein refer to non-random binding of a binding agent (target binding compound) such as a capture agent to an epitope on a predetermined antigen.

Binding agents (e.g., peptides) which specifically bind to a target are also referred to as having affinity for the target, or a binding site thereon. Typically, the binding agent binds with an affinity (KD) of approximately less than 10^{31 7} M, such as approximately less than 10⁻⁸ M, 10⁻⁹ M or 10⁻¹⁹ M or even lower.

The term “KD” as used herein refers to the dissociation equilibrium constant of a particular interaction between a binding agent such as a capture agent and its antigen. Typically, the disclosed binding agents bind to a target (e.g., CD8) with a dissociation equilibrium constant (KD) of less than approximately 10⁻⁷ M, such as less than approximately 10⁻⁸ M, 10⁻⁹ M or 10⁻¹⁹ M or even lower, for example, as determined using surface plasmon resonance (SPR) technology in a Biacore instrument using the antigen as the ligand and the capture agent as the analyte, and binds to the predetermined antigen with an affinity corresponding to a KD that is at least ten-fold lower, such as at least 100 fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the KD of the capture agent, so that when the KD of the capture agent very low (that is, the capture agent is highly specific), then the amount with which the affinity for the antigen is lower than the affinity for a non-specific antigen can be at least 10,000 fold.

The term “kd” (sec′) as used herein refers to the dissociation rate constant of a particular binding agent-antigen interaction. Said value is also referred to as the koff value.

The term “ka” (M-‘xsec ’) as used herein refers to the association rate constant of a particular binding agent-antigen interaction.

The term “KD” (M) as used herein refers to the dissociation equilibrium constant of a particular binding agent-antigen interaction.

The term “KA” (M-′) as used herein refers to the association equilibrium constant of a particular binding agent-antigen interaction and is obtained by dividing the ka by the kd.

The term “condition” as used herein refers generally to a disease, event, or a change in health status. A change in health status may be associated with a particular disease or event, in which case the change may occur simultaneously with or in advance of the disease or event. In those cases where the change in health status occurs in advance of a disease or event, the change in health status may serve as a predictor of the disease or event. For example, a change in health status may be an alteration in the expression level of a particular gene associated with a disease or event. Alternatively, a change in health status may not be associated with a particular disease or event.

The term “antibody” as used herein refers to a protein of the kind that is produced by activated B cells after stimulation by an antigen and can bind specifically to the antigen promoting an immune response in biological systems. Full antibodies typically consist of four subunits including two heavy chains and two light chains. The term antibody includes natural and synthetic antibodies, including but not limited to monoclonal antibodies, polyclonal antibodies or fragments thereof. Exemplary antibodies include IgA, IgD, IgG1, IgG2, IgG3, IgM and the like. Exemplary fragments include Fab, Fv, Fab′, F(ab′)2 and the like. A monoclonal antibody is an antibody that specifically binds to and is thereby defined as complementary to a single particular spatial and polar organization of another biomolecule which is termed an “epitope.” In some forms, monoclonal antibodies can also have the same structure. A polyclonal antibody refers to a mixture of different monoclonal antibodies. In some forms, polyclonal antibodies can be a mixture of monoclonal antibodies where at least two of the monoclonal antibodies binding to a different antigenic epitope. The different antigenic epitopes can be on the same target, different targets, or a combination. Antibodies can be prepared by techniques that are well known in the art, such as immunization of a host and collection of sera (polyclonal) or by preparing continuous hybridoma cell lines and collecting the secreted protein (monoclonal).

The term “stable” as used herein with regard to the disclosed peptides or pharmaceutical formulation thereof means that the agent or formulation maintains structural and functional integrity for a sufficient period of time to be useful in the methods described herein.

The term “synthetic” as used herein with regard to the disclosed peptides means that the capture agent has been generated by chemical rather than biological means. Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which can be homopolymeric tracts, short-period repeats, or regions enriched in one or more amino acids. Such low-complexity regions can be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity can be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantially identical” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

“Prodrug” is meant to indicate a compound that can be converted under physiological conditions or by solvolysis to a biologically active compound, such as the disclosed compounds. Thus, the term “prodrug” refers to a metabolic precursor of a compound that is pharmaceutically acceptable. A prodrug can be inactive when administered to a subject in need thereof, but is converted in vivo to an active compound. Prodrugs are typically rapidly transformed in vivo to yield the parent compound, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of a compound can be prepared by modifying functional groups present in the compound in such a way that the modifications are cleaved, either in routine manipulation or in vivo, to the parent compound. Prodrugs include compounds wherein a hydroxy, amino or mercapto group is bonded to any group that, when the prodrug of the compound is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds and the like.

The disclosed peptides are also meant to encompass all pharmaceutically acceptable forms of the peptides being isotopically-labelled by having one or more atoms replaced by an atom having a different atomic mass or mass number are intended to be encompas. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled peptides, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled peptides can generally be prepared by conventional techniques known to those skilled in the art or by processes analogous to those described in the Preparations and Examples as set out below using an appropriate isotopically-labeled reagent in place of the non-labeled reagent previously employed.

The disclosed peptides are also meant to encompass the in vivo metabolic products of the disclosed peptides. Such products can result from, for example, the oxidation, reduction, hydrolysis, amidation, esterification, and the like of the administered compound, primarily due to enzymatic processes. Accordingly, disclosed are compounds produced by a process comprising administering a compound to a mammal for a period of time sufficient to yield a metabolic product thereof. Such products are typically identified by administering a radiolabelled compound in a detectable dose to an animal, such as rat, mouse, guinea pig, monkey, or to human, allowing sufficient time for metabolism to occur, and isolating its conversion products from the urine, blood or other biological samples.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts. “Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound. As used herein, the term “solvate” refers to an aggregate that comprises one or more molecules of a compound with one or more molecules of solvent. The solvent can be water, in which case the solvate can be a hydrate. Alternatively, the solvent can be an organic solvent. Thus, the disclosed compounds may exist as a hydrate, including a monohydrate, dihydrate, hemihydrate, sesquihydrate, trihydrate, tetrahydrate and the like, as well as the corresponding solvated forms. The compound may be true solvates, while in other cases, the compound may merely retain adventitious water or be a mixture of water plus some adventitious solvent.

A “pharmaceutical composition” refers to a formulation of a compound and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a peptide as disclosed that, when administered to a mammal, preferably a human, is sufficient to effect treatment of a disease or condition in the mammal, preferably a human. The amount of a compound that constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” can be used interchangeably or can be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds (peptides) as disclosed, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. All such possible isomers, as well as their racemic and optically pure forms, are intended to be encompassed. Optically active (+) and (-), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. Various stereoisomers and mixtures thereof are contemplated and includes “enantiomers,” which refers to two stereoisomers whose molecules are nonsuperimposeable mirror images of one another.

A “tautomer” refers to a proton shift from one atom of a molecule to another atom of the same molecule. Tautomers of any said compounds are also contemplated.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular forms only and is not intended to be limiting.

In situ click chemistry (J. Am. Chem. Soc. 126:12809 (2004); Angew. CHem. Int. Ed. Engl. 44:116 (2004); Angew. Chem. Int. Ed. Engl. 45:1435 (2006)) is a technique in which a small molecule enzymatic inhibitor is separated into two moieties, each of which is then expanded into a small library—one containing acetylene functionalities, and the other containing azide groups. The enzyme itself then assembles the ‘best fit’ inhibitor from these library components by selectively promoting 1,3-dipolar cycloaddition between the acetylene and azide groups to form a triazole linkage (the ‘click’ reaction). The enzyme promotes the click reaction only between those library components that bind to the protein in the right orientation. The resultant inhibitor can exhibit far superior affinity characteristics relative to the initial inhibitor that formed the basis of the two libraries (Proc. Natl. Acad. Sci. USA 97:9367 (1981); J. Comput. Aided. Mol. Des. 16:741 (2002)).

Sequential in situ click chemistry extends the in situ click chemistry concept to enable the discovery of multiligand capture agents. This process was used previously to produce a triligand capture agent against the model protein carbonic anhydrase II (CAII) (Angew. Chem. Int. Ed. Engl. 48:4944 (2009)). Sequential in situ click chemistry has several advantages. First, structural information about the protein target is replaced by the ability to sample a very large chemical space to identify the ligand components of the capture agent. For example, an initial ligand can be identified by screening the protein against a large (>10⁶ element) one-bead-one-compound (OBOC) (Nature 354:83 (1991)) peptide library, where the peptides themselves can be comprised of natural, non-natural, and/or artificial amino acids. The resultant anchor ligand is then utilized in an in situ click screen, again using a large OBOC library, to identify a biligand binder. A second advantage is that the process can be repeated, so that the biligand is used as an anchor to identify a triligand, and so forth. The final capture agent can then be scaled up using relatively simple and largely automated chemistries, and it can be developed with a label, such as a biotin group, as an intrinsic part of its structure. This approach permits the exploration of branched, cyclic, and linear capture agent architectures. While many strategies for protein-directed multiligand assembly have been described (Science 274:1531 (1996); Proc. Natl. Acad. Sci. USA 97:9367 (2000)), most require detailed structural information on the target to guide the screening strategy, and most (such as the original in situ click approach), are optimized for low- diversity small molecule libraries.

In some forms, the cyclic peptides (also referred to herein as capture agents or binding agents) provided herein have a shelf-life of greater than six months, meaning that they are stable in storage for greater than six months. In some forms, the capture agents have a shelf-life of one year or greater, two years or greater, or more than three years. In some forms, the capture agents are stored as a lyophilized powder. In some forms, the capture agents provided herein have a longer shelf-life than a biologic binding to the same target, such as a target protein.

In some forms, the capture agents provided herein are stable at temperatures ranging from about −80° C. to about 120° C. In some forms, the capture agents are stable within a temperature range of −80° C. to −40° C.; −40° C. to −20° C.; −20° C. to 0° C.; 0° C. to 20° C.; 20° C. to 40° C.; 40° C. to 60° C.; 60° C. to 80° C.; and/or 80° C. to 120° C. In some forms, the capture agents provided herein are stable across a wider range of temperatures than a biologic binding to the same target, such as a target protein, and/or remain stable at a specific temperature for a longer time period than a biologic binding to the same target.

In some forms, the pH of a capture agent provided herein is in the range of about 3.0 to about 12.0. In some forms, the pH of the capture agent is in the range of about 5.0 to about 9.0. The pH of a capture agent can be adjusted to a physiologically compatible range using methods known in the art. For example, in some forms the pH of the capture agent can be adjusted to the range of about 6.5 to about 8.5.

In some forms, the capture agents provided herein are stable in blood serum for more than 12 hours. In some forms, the capture agents are stable in blood serum for more than 18 hours, more than 24 hours, more than 36 hours, more than 48 hours, or more than 96 hours. In some forms, the capture agents provided herein are stable for a longer period of time in blood serum than a biologic binding to the same target, such as a target protein.

In some forms, the capture agents provided herein can comprise one or more detection labels (reporter group), including for example biotin, copper-1,4,7,10-tetraazacyclododecane-1,4,7,10-tetraaceticacid (copper-DOTA), desferrioxamine B (DFO), a ligand for radiolabeling with ⁶⁸Ga, or other radiolabeled products that can include gamma emitters, proton emitters, positron emitters, tritium, or covered tags detectable by other methods (i.e., gadolinium) among others. In some forms, the capture agents provided herein comprise one or more detectable labels. In some forms, the label is copper-DOTA. In other forms, the detectable label is selected from ⁶⁴Cu DOTA, ⁶⁸Ga DOTA, ¹⁸F, ⁶⁴Cu,r, ⁸⁶Y, ^{94m}Tc, ^{110m}In, ¹¹C and ⁷⁶Br. In other forms, the detectable label is selected from ¹²³I, ¹³¹I, ⁶⁷Ga, ¹¹¹In and ⁹⁹mTc. In some forms, the label is a fluorescent label. In some forms, the cyclic peptide comprises a linkage to a reporter moiety, the reporter moiety selected from polyethylene glycol (PEG), biotin, thiol and fluorophores. For example, in some forms the fluorophores are selected from FAM, FITC, Cy5, TRITC, TAMRA.

Table 1 provides reporter moieties useful in various different applications of the cyclic peptides. Other useful reporter moieties can be derived by one of skill in the art.

TABLE 1
Reporter Moieties

| Application | Reporter |
|---|---|
| ELISA: microtiter plate | Biotin |
| ELISA: lateral flow test | Biotin |
| Immunoprecipitation (and | Biotin, thiol |
| other bead-based assays) | |
| Dot blot | Biotin |
| Cell-based assay | Biotin, fluorophore |
| IHC | Biotin, fluorophore |
| In vivo imaging: PET | Radioisotopes including ¹⁸F, |
| | ⁶⁸Ga, ⁶⁴Cu, ⁸⁹Zr, ¹²⁴I |
| In vivo imaging: SPECT | Radioisotopes including ¹¹¹In, |
| | ⁹⁰Y, ^{99m}Tc |
| In vivo imaging: MR | Gd³⁺ |

### Table 1. Reporter Moieties

Application Reporter

ELISA: microtiter plate Biotin

ELISA: lateral flow test Biotin

Immunoprecipitation (and other Biotin, thiol

bead-based assays)

Dot blot Biotin

Cell-based assay Biotin, fluorophore

IHC Biotin, fluorophore

In vivo imaging: PET Radioisotopes including ¹⁸F,

⁶⁸Ga, ⁶⁴Cu, ¹¹¹Zr, ¹²⁴I

In vivo imaging: SPECT Radioisotopes including ¹¹¹In,

⁹⁹Y, ⁹⁹mTc

In vivo imaging: MR Gd³⁺

In some forms, the capture agents provided herein can be modified to obtain a desired chemical or biological activity. Examples of desired chemical or biological activities include, without limitation, improved solubility, stability, bioavailability, detectability, or reactivity. Examples of specific modifications that can be introduced to a capture agent include, but are not limited to, cyclizing the capture agent through formation of a disulfide bond; modifying the capture agent with other functional groups or molecules. Similarly, a capture agent can be synthesized to bind to non-canonical or non-biological epitopes on proteins, thereby increasing their versatility. In some forms, the capture agent can be modified by modifying the synthesis blocks of the target-binding moieties before the coupling reaction.

Disclosed are pharmaceutical formulations comprising one or more of the capture agents provided herein. In some forms, these pharmaceutical formulations comprise one or more pharmaceutically acceptable carriers, excipients, or diluents. These carriers, excipients, or diluents can be selected based on the intended use and/or route of administration of the formulation.

Disclosed are kits comprising one or more of the capture agents disclosed herein. In some forms, the kits provided herein can further comprise instructions for suitable operational parameters in the form of a label or a separate insert. For example, the kit can have standard instructions informing a consumer/kit user how to wash the probe after a sample of plasma or other tissue sample is contacted on the probe.

It is understood that any form of the disclosed peptides can be independently combined with other forms and/or substituents of the disclosed peptides to produce forms not specifically set forth above. In addition, in the event that a list of substituents is listed for any particular variable in a particular form and/or claim, it is understood that each individual substituent can be deleted from the particular form and/or claim and that the remaining list of substituents will be considered to be within the scope of the disclosed compounds and methods.

For the purposes of administration, the disclosed peptides can be administered as a raw chemical or can be formulated as pharmaceutical compositions. Pharmaceutical compositions as disclosed can comprise a disclosed peptide and a pharmaceutically acceptable carrier, diluent or excipient. The disclosed peptide is present in the composition in an amount which is effective to treat a particular disease or condition of interest—that is, and preferably with acceptable toxicity to the patient. Activity of compounds of the peptides can be determined by one skilled in the art. Appropriate concentrations and dosages can be readily determined by one skilled in the art.

Administration of the disclosed compounds, or their pharmaceutically acceptable salts, in pure form or in an appropriate pharmaceutical composition, can be carried out via any of the accepted modes of administration of agents for serving similar utilities. The pharmaceutical compositions as disclosed can be prepared by combining a compound as disclosed with an appropriate pharmaceutically acceptable carrier, diluent or excipient, and can be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions as disclosed can be formulated so as to allow the active ingredients contained therein to be bioavailable upon administration of the composition to a patient. Compositions that will be administered to a subject or patient take the form of one or more dosage units, where for example, a tablet can be a single dosage unit, and a container of a compound as disclosed in aerosol form can hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound as disclosed, or a pharmaceutically acceptable salt thereof, for treatment of a disease or condition of interest in accordance with the teachings herein.

A pharmaceutical composition of the disclosed compounds can be in the form of a solid or liquid. In one aspect, the carrier(s) are particulate, so that the compositions are, for example, in tablet or powder form. The carrier(s) can be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition can be formulated into a powder, granule, compressed tablet, pill, capsule, chewing gum, wafer or the like form. Such a solid composition will typically contain one or more inert diluents or edible carriers. In addition, one or more of the following can be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it can contain, in addition to materials of the above type, a liquid carrier such as polyethylene glycol or oil.

The pharmaceutical composition can be in the form of a liquid, for example, an elixir, syrup, solution, emulsion or suspension. The liquid can be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent can be included.

The liquid pharmaceutical compositions as disclosed, whether they be solutions, suspensions or other like form, can include one or more of the following adjuvants: sterile diluents such as water for injection, saline solution, preferably physiological saline, Ringer′s solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a compound such that a suitable dosage will be obtained.

The pharmaceutical composition can be intended for topical administration, in which case the carrier can suitably comprise a solution, emulsion, ointment or gel base. The base, for example, can comprise one or more of the following: petrolatum, lanolin, polyethylene glycols, bee wax, mineral oil, diluents such as water and alcohol, and emulsifiers and stabilizers. Thickening agents can be present in a pharmaceutical composition for topical administration. If intended for transdermal administration, the composition can include a transdermal patch or iontophoresis device.

The pharmaceutical composition can be intended for rectal administration, in the form, for example, of a suppository, which will melt in the rectum and release the drug. The composition for rectal administration can contain an oleaginous base as a suitable nonirritating excipient. Such bases include, without limitation, lanolin, cocoa butter and polyethylene glycol.

The pharmaceutical composition can include various materials, which modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The pharmaceutical composition in solid or liquid form can include an agent that binds to the compound and thereby assists in the delivery of the compound. Suitable agents that can act in this capacity include a monoclonal or polyclonal antibody, a protein or a liposome.

The pharmaceutical composition can consist of dosage units that can be administered as an aerosol. The term aerosol is used to denote a variety of systems ranging from those of colloidal nature to systems consisting of pressurized packages. Delivery can be by a liquefied or compressed gas or by a suitable pump system that dispenses the active ingredients. Aerosols of compounds can be delivered in single phase, bi-phasic, or tri-phasic systems in order to deliver the active ingredient(s). Delivery of the aerosol includes the necessary container, activators, valves, subcontainers, and the like, which together can form a kit. One skilled in the art, without undue experimentation can determine preferred aerosols.

The pharmaceutical compositions can be prepared by methodology well known in the pharmaceutical art. For example, a pharmaceutical composition intended to be administered by injection can be prepared by combining a compound with sterile, distilled water so as to form a solution. A surfactant can be added to facilitate the formation of a homogeneous solution or suspension. Surfactants are compounds that non-covalently interact with the compound so as to facilitate dissolution or homogeneous suspension of the compound in the aqueous delivery system.

The compounds, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds as disclosed, or pharmaceutically acceptable derivatives thereof, can also be administered simultaneously with, prior to, or after administration of one or more other therapeutic agents. Such combination therapy includes administration of a single pharmaceutical dosage formulation which contains a compound as disclosed and one or more additional active agents, as well as administration of the compound and each active agent in its own separate pharmaceutical dosage formulation. For example, a compound and the other active agent can be administered to the patient together in a single oral dosage composition such as a tablet or capsule, or each agent administered in separate oral dosage formulations. Where separate dosage formulations are used, the compounds as disclosed and one or more additional active agents can be administered at essentially the same time, i.e., concurrently, or at separately staggered times, i.e., sequentially; combination therapy is understood to include all these regimens.

It is understood that in the present description, combinations of substituents and/or variables of the depicted formulae are permissible only if such contributions result in stable compounds.

It will also be appreciated by those skilled in the art that in the process described herein the functional groups of intermediate compounds can need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups can be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group can also be a polymer resin such as a Wang resin, Rink resin or a 2-chlorotrityl-chloride resin.

It will also be appreciated by those skilled in the art, although such protected derivatives of compounds may not possess pharmacological activity as such, they may be administered to a mammal and thereafter metabolized in the body to form compounds which are pharmacologically active. Such derivatives may therefore be described as “prodrugs.” All prodrugs of compounds as disclosed are contemplated.

Furthermore, all compounds as disclosed that exist in free base or acid form can be converted to their pharmaceutically acceptable salts by treatment with the appropriate inorganic or organic base or acid by methods known to one skilled in the art. Salts of the compounds can be converted to their free base or acid form by standard techniques.

The disclosed peptides can be prepared by procedures known to those of skill in the art. For example, the peptides can be prepared using standard solid-phase peptide synthesis techniques, and modifications thereof. Modified amino acids can be employed to incorporate amino acids comprising alkyne and/or azide moieties and/or alkene moieties useful for cyclization. Methods for cyclizing the peptides using azide/alkyne chemistry and Grubbs metathesis chemistry are well-known in the art. Such methods are described in more detail in the examples.

It is understood that one skilled in the art can make these compounds by similar methods or by combining other methods known to one skilled in the art. It is also understood that one skilled in the art would be able to make, in a similar manner as described below, other peptides not specifically illustrated in the examples below by using the appropriate starting components and modifying the parameters of the synthesis as needed. In general, starting components can be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein. Methods for Use of the peptides

Disclosed are methods for identification of cyclic peptides which are useful as binding agents for various targets. In general, the methods employ cyclic peptides, such as any of the cyclic peptides described herein above, in methods for identification of mono-, bi- and/or tri-ligand binding agents. Higher order binding agents (tetra, penta, and the like) are also contemplated.

In general, the disclosed methods can employ the cyclic peptides described herein. Accordingly, disclosed are methods for identifying a target binding compound (e.g., a protein capture agent), the method comprising

(a) providing a peptide library comprising a plurality of cyclic peptides;

(b) contacting the peptide library with a target or a synthetic epitope thereof, the target or a synthetic epitope thereof comprising a binding site and optionally an alkyne, azide or reporter moiety or combinations thereof, wherein the synthetic epitope is cross-linked;

(c) identifying a peptide library member with affinity for the binding site

In some forms, a method for identifying a target binding compound (e.g., a protein capture agent) is provided, the method comprising:

(a) providing a first peptide library comprising a plurality of first peptide library members, the first peptide library members optionally comprising an alkyne, azide or reporter moiety or combinations thereof;

(b) contacting the first peptide library with a target or a synthetic epitope thereof, the target or synthetic epitope thereof comprising a first binding site and optionally an alkyne, azide or reporter moiety or combinations thereof, wherein the synthetic epitope is cross-linked;

(c) identifying a first peptide library member with affinity for the first binding site and optionally modifying the first peptide library member to include an alkyne or azide moiety;

and optionally:

(d) providing a second peptide library comprising a plurality of second peptide library members, the second peptide library members comprising an azide or alkyne or both;

(e) contacting the second peptide library with a composition comprising the target or a synthetic epitope thereof and the first peptide library member of step C, wherein the synthetic epitope is cross-linked;

(f) forming a triazole-linked conjugate between the first peptide library member of step C and a second peptide library member, the second peptide library member having affinity for a second binding site on the target or a synthetic epitope thereof, wherein the synthetic epitope is cross-linked,

wherein the first peptide library, the second peptide library, or both, comprise cyclic peptides.

For purposes of clarity, it should be noted that steps (d)-(f) are optional and the above described method is not limited to methods which require conjugation of a second peptide. It is understood that when steps (d)-(f) are not performed, the first library comprises the cyclic peptide; however when steps (d)-(f) are performed the cyclic peptides can be a part of either the first, second or both libraries. It should also be emphasized that the methods are not limited to identification of mono or bi-ligand binding agents, and the methods described herein can be extrapolated to identification of tertiary, ternary and higher binding agents (e.g., by performing steps analogous to steps (d)-(f)). In general, any of the cyclic peptides described herein above can be employed in the above methods. Specific forms of the peptides useful in the some forms of the methods are illustrated herein.

Disclosed are peptides comprising an epitope, where the peptide is cross-linked, where the epitope corresponds to an epitope of a target, wherein the peptide does not include the entire target, and where the cross-link is a disulfide, preferably a disulfide that naturally occurs in the target or between added or substitute cysteines. Also disclosed are methods of preparing a peptide comprising an epitope, where the epitope corresponds to an epitope of a target, where the peptide does not include the entire target, the method comprising cross-linking the peptide, where the cross-link is a disulfide, preferably a disulfide that naturally occurs in the target or between added or substitute cysteines.

In some forms, the target is a target protein. In some forms, the target protein is CD8. In some forms, the epitope comprises amino acids 24 to 33 of CD8. In some forms, the disulfide is between amino acids C22 and C33 of CD8. In some forms, the peptide comprises amino acids 21 to 35 of CD8. In some forms, the peptide further comprises an alkyne moiety, an azide moiety, a reporter moiety, or combinations thereof. In some forms, the azide moiety and the alkyne moiety are comprised in an artificial amino acid. In some forms, the artificial amino acid is propargylglycine (Fra). In some forms the artificial amino acid replaces L26 of CD8. In some forms, the reporter moiety is biotin.

In some forms, the peptide further comprises an alkyne moiety, an azide moiety, a reporter moiety, or combinations thereof, where the epitope comprises a phosphorylated amino acid, where the azide moiety and the alkyne moiety of the target peptide are metalorganic molecules that selectively bind to the phospho group on the phosphorylated amino acid. In some forms, the target is a target protein, where the target protein is CD8, where the phosphorylated amino acid is Ser235 or Ser236 of CD8. In some forms, the metalorganic molecule comprises the reporter moiety. In some forms, the reporter moiety is biotin. Provided herein in some forms are methods of using the capture agents disclosed

herein to identify, detect, quantify, and/or separate targets, such as target proteins, in a biological sample. The capture agents disclosed herein can serve as a drop-in replacement for monoclonal antibodies in biochemical assays. Therefore, in some forms the methods provided herein utilize an immunoassay, with the capture agent replacing an antibody or its equivalent. In some forms, the immunoassay can be a Western blot, pull-down assay, dot blot, or ELISA.

A biological sample for use in the methods provided herein can be selected from the group consisting of organs, tissue, bodily fluids, and cells. Where the biological sample is a bodily fluid, the fluid can be selected from the group consisting of blood, blood serum, plasma, urine, sputum, saliva, stool, spinal fluid, cerebral spinal fluid, lymph fluid, skin secretions, respiratory secretions, intestinal secretions, genitourinary tract secretions, tears, and milk.

Disclosed are methods of identifying, detecting, quantifying, and/or localizing a target in vivo. In some forms, the capture agents can be used as an imaging agent. In these forms, the capture agents can comprise one or more detection labels as discussed above.

Disclosed are methods of using the capture agents disclosed herein to inhibit a target activity, such as a target protein activity. In some forms, the capture agents inhibit target activity by blocking binding of the target to its native substrate.

Provided herein in some forms are methods of using the capture agents disclosed herein to diagnose and/or classify (e.g., stage) a condition associated with increased target expression and/or activity, such as target protein expression and/or activity. In some forms, these methods comprise (a) obtaining a biological sample from a subject;

(b) measuring the presence or absence of target in the sample with the capture agent; (c) comparing the levels of target to a predetermined control range for target; and (d) diagnosing a condition associated with increased target expression based on the difference between target levels in the biological sample and the predetermined control.

In some forms of the diagnosis and/or classification methods provided herein, the capture agents can be used to diagnose a change in health status in a subject, wherein the change in health status is a predictor of a disease or event. In some forms, the methods can be utilized to predict the development of a disease or event in a subject who does not yet exhibit any symptoms of the disease or event. In some forms, the change in health status can be an increase in target levels, such as target protein levels.

Disclosed are methods of treating a condition associated with increased target expression and/or activity in a subject in need thereof by administering a therapeutically effective amount of one or more of the capture agents or pharmaceutical formulations disclosed herein. In some forms, the capture agent(s) can be linked to one or more additional therapeutic agents, including for example a chemotherapeutic agent. In preferred forms, the capture agent is administered as a pharmaceutical composition.

A capture agent or pharmaceutical formulation can be administered to a patient in need of treatment via any suitable route. Routes of administration can include, for example, parenteral administration (including subcutaneous, intramuscular, intravenous, by means of, for example a drip patch). Further suitable routes of administration include (but are not limited to) oral, rectal, nasal, topical (including buccal and sublingual), infusion, vaginal, intradermal, intraperitoneally, intracranially, intrathecal and epidural administration or administration via oral or nasal inhalation, by means of, for example a nebulizer or inhaler, or by an implant.

A capture agent or pharmaceutical formulation can also be administered via microspheres, liposomes, other microparticulate delivery systems or sustained release formulations placed in certain tissues including blood. Suitable examples of sustained release carriers include semi-permeable polymer matrices in the form of shared articles, e.g., suppositories or microcapsules. Examples of the techniques and protocols mentioned above and other techniques and protocols which can be used in accordance with the disclosed compounds, compositions, and methods can be found in Remington′s Pharmaceutical Sciences, 18th edition, Gennaro, A. R., Lippincott Williams & Wilkins; 20th edition (Dec. 15, 2000) ISBN 0- 912734-04-3 and Pharmaceutical Dosage Forms and Drug Delivery Systems; Ansel, N. C. et al. 7th Edition ISBN 0-683305-72-7, the entire disclosures of which are herein incorporated by reference.

Disclosed is the use of the disclosed capture agents in the preparation of a medicament for treating a condition associated with increased target expression and/or activity, such as a target protein expression and/or activity.

Also disclosed are methods of detecting a target, such as a target protein, in a sample, can comprise replacing an antibody or its equivalent in a cell-based or an immunoassay with any of the foregoing cyclic peptides. In some forms, the immunoassay is a Western blot, a pull-down assay, a dot blot or an ELISA.

Also disclosed are methods for inhibiting activity of a protein in a subject, the method comprising administering an effective amount of any of the foregoing cyclic peptides to a subject in need thereof.

Also disclosed are methods of purifying a target, the method comprising immobilizing any of the foregoing cyclic peptides in a column based format, contacting the column with a matrix containing the target, washing the column, and eluting the target.

Methods for imaging are also provided. For example, disclosed are methods of imaging in vivo target expression, the method comprising:

(a) providing any of the foregoing cyclic peptides, wherein SEQ is a peptide sequence having affinity for a location on or near a target expressing site in a subject, and modifying the cyclic peptide to include a small-molecule positron-emission-tomography ligand (PET ligand);

(b) administering the cyclic peptide of step (a) to the subject;

(c) measuring the positron emission from the PET ligand at a first time;

(d) measuring the positron emission from the PET ligand at a second time; and

(e) comparing the positron emission from the PET ligand at the first and second times.

In some forms of the foregoing, the PET ligand comprises a moiety selected from ⁶⁴Cu DOTA, ⁶⁸Ga DOTA, ⁶⁸Ga NOTA, ¹⁸F, ⁶⁴Cu, ⁶⁸Ga, ⁸⁹Zr , ¹²⁴I, ⁸⁶Y, ⁹⁴mTc, ^{110m}In, ¹¹C and ⁷⁶Br.

In some forms, the imaging method is a method of imaging in vivo target expression, the method comprising:

(a) providing any of the foregoing cyclic peptides, wherein SEQ is a peptide sequence having affinity for a location on or near a target expressing site in a subject, and modifying the cyclic peptide to include a small-molecule single-photon-emission-computed-tomography ligand (SPECT ligand);

(b) administering the cyclic peptide of step a) to the subject;

(c) measuring the photon emission from the SPECT ligand at a first time;

(d) measuring the photon emission from the SPECT ligand at a second time; and

(e) comparing the photon emission from the SPECT ligand at the first and second times.

In some forms of the foregoing, the SPECT ligand comprises a moiety selected from ¹¹¹In DOTA, ⁹⁰Y DOTA, ¹¹¹In, ⁹⁰Y and ⁹⁹mTc.

In some forms, a method of imaging in vivo target expression is provided, the method comprising:

(a) providing any of the foregoing cyclic peptides, wherein SEQ is a peptide sequence having affinity for a location on or near a target expressing site in a subject, and modifying the cyclic peptide to include a magnetic resonance ligand (MR ligand);

(b) administering the cyclic peptide of step a) to the subject;

(c) measuring the magnetic resonance from the MR ligand at a first time;

(d) measuring the magnetic resonance from the MR ligand at a second time; and

(e) comparing the magnetic resonance from the MR ligand at the first and second times.

In some forms, the MR ligand comprises Gd³⁺.

In some forms of the foregoing methods, the cyclic peptide comprises a linkage to a reporter moiety, the reporter moiety selected from polyethylene glycol (PEG), biotin, thiol and fluorophores. For example, in some forms the fluorophores are selected from FAM, FITC, Cy5, TRITC, TAMRA.

Human CD8 (cluster of differentiation 8) is a transmembrane glycoprotein that serves as a co-receptor for the T cell receptor (TCR). CD8 binds to the major peptide-histocompatibility complexes (pMHC) on antigen-presenting cells to result in antigen-specific activation of naive or effector CD8+ T cells (Kroger et al Immunology 2007). CD8+ cytotoxic T lymphocytes (CTLs) are an important part of the immune system because properly activated and non-inhibited CTLs can recognize tumor-associated antigens and kill tumor cells.

Immunotherapy is a type of cancer treatment that harnesses the immune system to combat cancer. For solid tumors, immunotherapy has had the greatest success in easily biopsied cancers such as melanoma and lymphoma. For those patients, pre-existing CD8+ T cells located at the invasive tumor margin were found to be associated with expression of the PD-1/PD-L1 immune inhibitory axis and may predict response to therapy (Tumeh et al. Nature 2014; doi: 10.1038/nature13954). As immunotherapy is extended to additional cancers, the ability to detect CD8+ T cells in vivo has tremendous promise for patient stratification, as well as promise for following therapy responses.

There remains a need for sensitive and selective tools for CD8 detection.

Disclosed are epitope-targeted macrocyclic peptide ligands and capture agents having an affinity for CD8. The ligands and capture agents are designed to bind to specific synthetic epitopes of CD8 in a manner reminiscent of monoclonal antibodies (mAbs), and were developed by in situ click screening of one-bead-one-compound (OBOC) peptide libraries. In some forms, the ligands and capture agents comprise cyclic peptides. Cyclic peptides have the ability to display protein-like epitopes with restricted conformational flexibility and thus often display enhanced bioavailability, increased stability towards metabolic degradation, and superior binding affinities as compared to their linear counterparts.

Disclosed are stable, synthetic capture agent that specifically binds CD8, wherein the capture agent comprises a first ligand having affinity for a first epitope on CD8, a second ligand having affinity for a second epitope on CD8, and a linker covalently connecting the first ligand to the second ligand. Also disclosed are compositions comprising one or more synthetic capture agents, as described herein, that specifically bind CD8.

In some forms, the first epitope is 5 to 30 amino acids long. In some forms, the first epitope is 8 to 20 amino acids long. In some forms, the first epitope is 7 to 13 amino acids long. In some forms, the first epitope is at most, 5, 6, 7, 8, 9, 10, 11, 12, 13, 14, 15, 16, 17, 18, 19 or 20 amino acids long. In some forms, the first epitope comprises the sequence KCQVLLSNPTSGCSW (SEQ ID NO:1; epitope B2; K21-W35). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, L26 is replaced with an azide-containing amino acid residue.

In some forms, the first and second epitopes are located in the CD8a ectodomain.

In some forms, the first and/or second epitopes are synthetic, having at least 90% homology to the corresponding natural epitope of the target protein, and having at least one amino acid comprising an azide or an acetylene group.

In some forms, the first ligand binds the first epitope (or a synthetic version thereof) in isolation and the second ligand binds the second epitope (or a synthetic version thereof) in isolation. In some forms, the first ligand and the second ligand cooperatively bind the first and second epitopes of CD8, respectively.

In some forms, the epitope can comprise the sequence epitope A SQFRVSPLDR (SEQ ID NO:2; epitope A; S1-R10). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, an azide-containing amino acid residue is added ahead of S1.

In some forms, the epitope can comprise the sequence epitope B VLLSNPTSGC (SEQ ID NO:3; epitope B; V24-C33). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, an azide-containing amino acid residue is added ahead of V24.

In some forms, the epitope can comprise the sequence KAAEGLDTQRFSGKRLGDTF (SEQ ID NO:4; epitope C; K58-F77). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, R67 is replaced with an azide-containing amino acid residue.

In some forms, the epitope can comprise the sequence FSGKRLGDTFVLTLSD (SEQ ID NO:5; epitope D; F68-D83). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, G74 is replaced with an azide-containing amino acid residue.

In some forms, the epitope can comprise the sequence SALSNSIMYFSHFVPV (SEQ ID NO:6; epitope E; S95-V110). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, M102 is replaced with an azide-containing amino acid residue.

In some forms, the epitope can comprise the sequence FQPRGAAASPTFL (SEQ ID NO:7; epitope F; F37-L49). For ligand coupling to the epitope, one of the amino acids can be replaced in the epitope with an azide-containing amino acid residue (for example, Az1, Az2, Az4, or Az8, where the number refers to the number of carbons in the azide-terminated side chain).

In some forms, the epitope can comprise the sequence LYLSQNKPKAA (SEQ ID NO:8; epitope G; L50-A60). For ligand coupling to the epitope, one of the amino acids can be replaced in the epitope with an azide-containing amino acid residue (for example, Az1, Az2, Az4, or Az8, where the number refers to the number of carbons in the azide-terminated side chain).

In some forms, the epitope can comprise the sequence SQFRVSPLDRTWNLGETVELKSQVL (SEQ ID NO:97; epitope A2; S1-L25 with C22 replaced with S). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, N13 is replaced with an azide-containing amino acid residue.

In some forms, the epitope can comprise the sequence RFSGKRLGDTFVLTLSD (SEQ ID NO:98; epitope H; R67-D83). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, G74 is replaced with an azide-containing amino acid residue.

In some forms, the epitope can comprise the sequence QNKPKAAEGLDTQRF (SEQ ID NO:99; epitope J; Q54-F68). For ligand coupling to the epitope, one of the amino acids can be replaced in the epitope with an azide-containing amino acid residue (for example, Az1, Az2, Az4, or Az8, where the number refers to the number of carbons in the azide-terminated side chain). Preferably, L63 is replaced with an azide-containing amino acid residue.

In some forms, the term “CD8” as used herein refers to human CD8. In some forms, CD8 comprises one of the following amino acid sequence or an amino acid sequence substantially identical to it.

Sequence of the ectodomain for human CD8a (SEQ ID NO:75):

SQFRVSPLDR TWNLGETVEL KCQVLLSNPT SGCSWLFQPR
GAAASPTFLL YLSQNKPKAA EGLDTQRFSG KRLGDTFVLT
LSDFRRENEG YYFCSALSNS IMYFSHFVPV FLPAKPTTTP
APRPPTPAPT IASQPLSLRP EACRPAAGGA
VHTRGLDFAC D

Sequence of the ectodomain for mouse CD8a (SEQ ID NO:76):

KPQAPELRIF PKKMDAELGQ KVDLVCEVLG SVSQGCSWLF
QNSSSKLPQP TFVVYMASSH NKITWDEKLN SSKLFSAMRD
TNNKYVLTLN KFSKENEGYY FCSVISNSVM YFSSVVPVLQ
KVNSTTTKPV LRTPSPVHPT GTSQPQRPED CRPRGSVKGT
GLDFACDIY

In some forms, provided herein is a stable, synthetic capture agent that specifically binds CD8, wherein the capture agent comprises two or more “anchor” ligands (also referred to as simply “ligands” herein) and a linker and wherein the ligands selectively bind CD8.

In some forms, a ligand comprises one or more polypeptides or peptides. In some forms, a target-binding moiety comprises one or more peptides comprising D-amino acids, L-amino acids, and/or amino acids substituted with functional groups selected from the group consisting of substituted and unsubstituted alkyl, substituted and unsubstituted azido, substituted and unsubstituted alkynyl, substituted and unsubstituted biotinyl, substituted and unsubstituted azidoalkyl, substituted and unsubstituted polyethyleneglycolyl, and substituted and unsubstituted 1,2,3-triazole.

In some forms, the ligands are linked to one another via a covalent linkage through a linker. In some forms, the ligand and linker are linked to one another via an amide bond or a 1,4-disubstituted-1,2,3-triazole linkage as shown below:

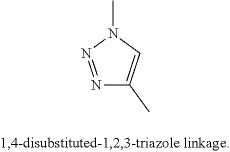

In those forms where the ligands and linker are linked to one another via a 1,4-disubstituted-1,2,3-triazole linkage, the 1,4-disubstituted-1,2,3-triazole linkage can be formed by Cu-Catalyzed Azide/Alkyne Cycloaddition (CuAAC).

In some forms, the ligands and linker are linked to one another by a Tz4 linkage having the following structure:

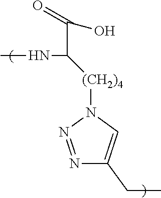

In some forms, the ligands and linker are linked to one another by a Tz5 linkage having the following structure:

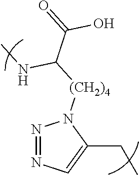

In those forms wherein one or more of the ligands and linker are linked to one another via amide bonds, the amide bond can be formed by coupling a carboxylic acid group and an amine group in the presence of a coupling agent (e.g., 0-(7-azabenzotriazol-1-yl)-N,N,N′,N-tetramethyluronium hexafluorophosphate (HATU), N-hydroxy-7-aza-benzotriazole (HOAt), or diisopropylethylamine (DIEA) in DMF).

In some forms, the capture agents provided herein are stable across a range of reaction conditions and/or storage times. A capture agent that is “stable” as used herein maintains the ability to specifically bind to a target protein. In some forms, the capture agents provided herein are more stable than an antibody binding to the same target protein under one or more reaction and/or storage conditions. For example, in some forms, the capture agents provided herein are more resistant to proteolytic degradation than an antibody binding to the same target protein.

In some forms, the capture agents provided herein have a shelf-life of greater than six months, meaning that they are stable in storage for greater than six months. In some forms, the capture agents have a shelf-life of one year or greater, two years or greater, or more than three years. In some forms, the capture agents are stored as a lyophilized powder. In some forms, the capture agents provided herein have a longer shelf-life than an antibody binding to the same target protein.

In some forms, the capture agents provided herein are stable at temperatures ranging from about −80° to about 120° C. In some forms, the capture agents are stable within a temperature range of −80° to −40° C.; −40° to −20° C.; −20° to 0° C.; 0° to 20° C.; 20° to 40° C.; 40° to 60° C.; 60° to 80° C.; and/or 80° to 120° C. In some forms, the capture agents provided herein are stable across a wider range of temperatures than an antibody binding to the same target protein, and/or remain stable at a specific temperature for a longer time period than an antibody binding to the same target protein.

In some forms, the capture agents provided herein are stable at a pH range from about 3.0 to about 8.0. In some forms, the range is about 4.0 to about 7.0. In some forms, the range is about 7.0 to about 8.0.

In some forms, the capture agents provided herein are stable in human serum for more than 12 hours. In some forms, the capture agents are stable in human serum for more than 18 hours, more than 24 hours, more than 36 hours, or more than 48 hours. In some forms, the capture agents provided herein are stable for a longer period of time in human serum than an antibody binding to the same target protein. In some forms, the capture agents are stable as a powder for two months at a temperature of about 60° C. In some forms, the capture agents provided herein can comprise one or more detection labels, including for example biotin, copper-1,4,7,10-tetraazacyclododecane-1,4,7,10-tetraacetic acid (copper-DOTA), ⁶⁴Cu DOTA, ⁶⁸Ga DOTA, ¹⁸F, ⁶⁴Cu, ⁶⁸Ga, ⁸⁹Zr, ¹²⁴Im ⁸⁶Y, ^{94m}Tc, ^{110m}In, 11C, ⁷⁶Br, ¹²³I, ¹³¹I, ⁶⁷Ga, ¹¹¹In and ^{99m}Tc, or other radiolabeled products that may include gamma emitters, proton emitters, positron emitters, tritium, or covered tags detectable by other methods (i.e., gadolinium) among others. In some forms, the detection label is ¹⁸F. In some forms, the capture agents can be modified to be used as imaging agents. The imaging agents can be used as diagnostic agents.

In some forms, the capture agents provided herein can be modified to obtain a desired chemical or biological activity. Examples of desired chemical or biological activities include, without limitation, improved solubility, stability, bioavailability, detectability, or reactivity. Examples of specific modifications that can be introduced to a capture agent include, but are not limited to, cyclizing the capture agent through formation of a disulfide bond; modifying the capture agent with other functional groups or molecules. Similarly, a capture agent can be synthesized to bind to non-canonical or non-biological epitopes on proteins, thereby increasing their versatility. In some forms, the capture agent can be modified by modifying the synthesis blocks of the target-binding moieties before the coupling reaction.

In some forms, the CD8 capture agents provided herein are stable across a wide range of temperatures, pH values, storage times, storage conditions, and reaction conditions, and in some forms, the capture agents are more stable than a comparable antibody or biologic. In some forms, the capture agents are stable in storage as a lyophilized powder. In some forms, the capture agents are stable in storage at a temperature of about -80° C. to about 60° C. In some forms, the capture agents are stable at room temperature. In some forms, the capture agents are stable in human serum for at least 24 hours. In some forms, the capture agents are stable at a pH in the range of about 3 to about 12. In some forms, the capture agents are stable as a powder for two months at a temperature of about 60° C.

For detection of CD8 in solution, a capture agent as described herein can be detectably labeled, then contacted with the solution, and thereafter formation of a complex between the capture agent and the CD8 target can be detected. As an example, a fluorescently labeled capture agent can be used for in vitro CD8 detection assays, wherein the capture agent is added to a solution to be tested for CD8 under conditions allowing binding to occur. The complex between the fluorescently labeled capture agent and the CD8 target can be detected and quantified by, for example, measuring the increased fluorescence polarization arising from the complex-bound peptide relative to that of the free peptide.

Alternatively, a sandwich-type “ELISA” assay can be used, wherein a capture agent is immobilized on a solid support such as a plastic tube or well, then the solution suspected of containing CD8 is contacted with the immobilized binding moiety, non-binding materials are washed away, and complexed polypeptide is detected using a suitable detection reagent for recognizing CD8.

For detection or purification of soluble CD8 from a solution, capture agents as disclosed can be immobilized on a solid substrate such as a chromatographic support or other matrix material, then the immobilized binder can be loaded or contacted with the solution under conditions suitable for formation of a capture agent/CD8 complex. The non-binding portion of the solution can be removed and the complex can be detected, for example, using an anti-CD8 antibody, or an anti-binding polypeptide antibody, or the CD8 can be released from the binding moiety at appropriate elution conditions.

A particularly preferred use for the disclosed capture agents is for creating visually readable images of CD8 or CD8-expressing cells in a biological fluid, such as, for example, in human serum. The CD8 capture agents disclosed herein can be converted to imaging reagents by conjugating the capture agents with a label appropriate for diagnostic detection. Preferably, a capture agent exhibiting much greater specificity for CD8 than for other serum proteins is conjugated or linked to a label appropriate for the detection methodology to be employed. For example, the capture agent can be conjugated with or without a linker to a paramagnetic chelate suitable for Magnetic Resonance Imaging (MRI), with a radiolabel suitable for x-ray, Positron Emission Tomography (PET), Single Photon Emission Computed Tomography (SPECT) or scintigraphic imaging (including a chelator for a radioactive metal), with an ultrasound contrast agent (e.g., a stabilized microbubble, a microballoon, a microsphere or what has been referred to as a gas filled “liposome”) suitable for ultrasound detection, or with an optical imaging dye.

In some forms, rather than directly labeling a capture agent with a detectable label or radiotherapeutic construct, one or more peptides or constructs as disclosed can be conjugated with for example, avidin, biotin, or an antibody or antibody fragment that will bind the detectable label or radiotherapeutic.

The CD8 capture agents described herein can advantageously be conjugated with a paramagnetic metal chelate in order to form a contrast agent for use in MRI.

Preferred paramagnetic metal ions have atomic numbers 21-29, 42, 44, or 57-83. This includes ions of the transition metal or lanthanide series which have one, and more preferably five or more, unpaired electrons and a magnetic moment of at least 1.7 Bohr magneton. Preferred paramagnetic metals include, but are not limited to, chromium (III), manganese (II), manganese (III), iron (II), iron (III), cobalt (II), nickel (II), copper (II), praseodymium (III), neodymium (III), samarium (III), gadolinium (III), terbium (III), dysprosium (III), holmium (III), erbium (III), europium (III) and ytterbium (III), chromium (III), iron (III), and gadolinium (III). The trivalent cation, Gd3+, is particularly preferred for MRI contrast agents, due to its high relaxivity and low toxicity, with the further advantage that it exists in only one biologically accessible oxidation state, which minimizes undesired metabolysis of the metal by a patient. Another useful metal is Cr3+, which is relatively inexpensive. Gd(III) chelates have been used for clinical and radiologic MR applications since 1988, and approximately 30% of MRI exams currently employ a gadolinium-based contrast agent.

The paramagnetic metal chelator is a molecule having one or more polar groups that act as a ligand for, and complex with, a paramagnetic metal. Suitable chelators are known in the art and include acids with methylene phosphonic acid groups, methylene carbohydroxamine acid groups, carboxyethylidene groups, or carboxymethylene groups. Examples of chelators include, but are not limited to, diethylenetriaminepentaacetic acid (DTPA), 1,4,7,10-tetraazacyclo-tetradecane-1,4,7,10-tetraacetic acid (DOTA), 1-substituted 1,4,7,-tricarboxymethyl-1,4,7,10-teraazacyclododecane (DO3A), ethylenediaminetetraacetic acid (EDTA), and 1,4,8,11-tetra-azacyclotetradecane-1,4,8,11-tetraacetic acid (TETA). Additional chelating ligands are ethylene bis-(2-hydroxy-phenylglycine) (EHPG), and derivatives thereof, including 5-CI-EHPG, 5-Br-EHPG, 5-Me-EHPG, 5-t-Bu-EHPG, and 5-sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl DTPA; bis-2 (hydroxybenzyl)-ethylene-diaminediacetic acid (HBED) and derivatives thereof; the class of macrocyclic compounds which contain at least 3 carbon atoms, more preferably at least 6, and at least two heteroatoms (0 and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11-tetraazacyclotetradecane-1,4,8,11-(methyl tetraacetic acid); derivatives of 1,3-propylene-diaminetetraacetic acid (PDTA) and triethylenetetraaminehexaacetic acid (TTNA); derivatives of 1,5,10-N,N′,N″-tris(2,3-dihydroxybenzoyl)-tricatecholate (LICAM); and 1,3,5-N,N′,N″-tris(2,3-dihydroxybenzoyl)aminomethylbenzene (MECAM). A preferred chelator for use in the disclosed compounds, compositions, and methods is DTPA, and the use of DO3A is particularly preferred. Examples of representative chelators and chelating groups are described in WO 98/18496, WO 86/06605, WO 91/03200, WO 95/28179, WO 96/23526, WO 97/36619, PCT/US98/01473, PCT/US98/20182, and U.S. Pat. No. 4,899,755, U.S. Pat. No. 5,474,756, U.S. Pat. No. 5,846,519 and U.S. Pat. No. 6,143,274, all of which are hereby incorporated by reference.

In accordance with the present disclosure, the chelator of the MRI contrast agent is coupled to the CD8 capture agent. The positioning of the chelate should be selected so as not to interfere with the binding affinity or specificity of the CD8 capture agent. The chelate also can be attached anywhere on the capture agent.

In general, the CD8 capture agent can be bound directly or covalently to the metal chelator (or other detectable label), or it can be coupled or conjugated to the metal chelator using a linker, which can be, without limitation, amide, urea, acetal, ketal, double ester, carbonyl, carbamate, thiourea, sulfone, thioester, ester, ether, disulfide, lactone, imine, phosphoryl, or phosphodiester linkages; substituted or unsubstituted saturated or unsaturated alkyl chains; linear, branched, or cyclic amino acid chains of a single amino acid or different amino acids (e.g., extensions of the N- or C-terminus of the CD8 binding moiety); derivatized or underivatized polyethylene glycols (PEGs), polyoxyethylene, or polyvinylpyridine chains; substituted or unsubstituted polyamide chains; derivatized or underivatized polyamine, polyester, polyethylenimine, polyacrylate, poly(vinyl alcohol), polyglycerol, or oligosaccharide (e.g., dextran) chains; alternating block copolymers; malonic, succinic, glutaric, adipic and pimelic acids; caproic acid; simple diamines and dialcohols; any of the other linkers disclosed herein; or any other simple polymeric linkers known in the art (see, for example, WO 98/18497 and WO 98/18496). Preferably the molecular weight of the linker can be tightly controlled. The molecular weights can range in size from less than 100 to greater than 1000. Preferably the molecular weight of the linker is less than 100. In addition, it can be desirable to utilize a linker that is biodegradable in vivo to provide efficient routes of excretion for the disclosed imaging reagents. Depending on their location within the linker, such biodegradable functionalities can include ester, double ester, amide, phosphoester, ether, acetal, and ketal functionalities.

In general, known methods can be used to couple the metal chelate and the CD8 capture agent using such linkers (WO 95/28967, WO 98/18496, WO 98/18497 and discussion therein). The CD8 binding moiety can be linked through an N- or C-terminus via an amide bond, for example, to a metal coordinating backbone nitrogen of a metal chelate or to an acetate arm of the metal chelate itself. The present disclosure contemplates linking of the chelate on any position, provided the metal chelate retains the ability to bind the metal tightly in order to minimize toxicity.

MRI contrast reagents prepared according to the disclosures herein can be used in the same manner as conventional MRI contrast reagents. Certain MR techniques and pulse sequences can be preferred to enhance the contrast of the site to the background blood and tissues. These techniques include (but are not limited to), for example, black blood angiography sequences that seek to make blood dark, such as fast spin echo sequences (Alexander, A. et al., 1998. Magn. Reson. Med., 40: 298-310) and flow-spoiled gradient echo sequences (Edelman, R. et al., 1990. Radiology, 177: 45-50). These methods also include flow independent techniques that enhance the difference in contrast, such as inversion-recovery prepared or saturation-recovery prepared sequences that will increase the contrast between CD8-expressing tissue and background tissues. Finally, magnetization transfer preparations also can improve contrast with these agents (Goodrich, K. et al., 1996. Invest. Radia, 31: 323-32).

The labeled reagent is administered to the patient in the form of an injectable composition. The method of administering the MRI contrast agent is preferably parenterally, meaning intravenously, intraarterially, intrathecally, interstitially, or intracavitarilly. For imaging CD8-expressing tissues, such as tumors, intravenous or intraarterial administration is preferred. For MRI, it is contemplated that the subject will receive a dosage of contrast agent sufficient to enhance the MR signal at the site CD8 expression by at least 10%. After injection with the CD8 capture agent containing MRI reagent, the patient is scanned in the MRI machine to determine the location of any sites of CD8 expression. In therapeutic settings, upon identification of a site of CD8 expression (e.g., fluid or tissue), an anti-cancer agent (e.g., inhibitors of CD8) can be immediately administered, if necessary, and the patient can be subsequently scanned to visualize viral load.

The disclosed CD8 capture agents can be conjugated with a radionuclide reporter appropriate for scintigraphy, SPECT, or PET imaging and/or with a radionuclide appropriate for radiotherapy. Constructs in which the CD8 capture agents are conjugated with both a chelator for a radionuclide useful for diagnostic imaging and a chelator useful for radiotherapy are contemplated.

For use as a PET agent a disclosed capture agent can be complexed with one of the various positron emitting metal ions, such as ⁵¹Mn, ⁵²Fe, ⁶⁰Cu, ⁶⁸Ga, ⁷²As, ⁹⁴mTc, or ¹¹⁹In. The disclosed binding moieties can also be labeled by halogenation using radionuclides such as ¹⁸F, ¹²⁴I, ¹²⁵I, ¹³¹I, ¹²³I, ⁷⁷Br, and ⁷⁶Br. Preferred metal radionuclides for scintigraphy or radiotherapy include ⁹⁹mTc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ^{117m}Sn, ¹⁴⁹Pm, ¹⁶¹Tb, ¹⁷⁷Lu, ¹⁹⁸Au and ¹⁹⁹Au. The choice of metal will be determined based on the desired therapeutic or diagnostic application. For example, for diagnostic purposes the preferred radionuclides include ⁶⁴Cu, ⁶⁷Ga, ⁶⁸Ga, ⁹⁹mTc, and ¹¹¹In. For therapeutic purposes, the preferred radionuclides include ⁶⁴Cu, ⁹⁰Y, ¹⁰⁵Rh, ¹¹¹In, ¹¹⁷mSn, ¹⁴⁹Pm, ¹⁵³Sm, ¹⁶¹Tb, ¹⁶⁶Tb, ¹⁶⁶Dy, ¹⁶⁶Ho, ¹⁷⁵Yb, ¹⁷⁷Ln, ^{186/188}Re, and ¹⁹⁹Au. ^{99m}Tc is useful for diagnostic applications because of its low cost, availability, imaging properties, and high specific activity. The nuclear and radioactive properties of 99mTc make this isotope an ideal scintigraphic imaging agent. This isotope has a single photon energy of 140 keV and a radioactive half-life of about 6 hours, and is readily available from a ⁹⁹Mo-⁹⁹mTc generator. ¹⁸F, 4-[¹⁸F]fluorobenzaldehyde (¹⁸FB), Al[¹⁸F]-NOTA, ⁶⁸Ga-DOTA, and ⁶⁸Ga-NOTA are typical radionuclides for conjugation to CD8 capture agents for diagnostic imaging.

The metal radionuclides can be chelated by, for example, linear, macrocyclic, terpyridine, and N₃S, N₂S₂, or N₄ chelants (see also, U.S. Pat. No. 5,367,080, U.S. Pat. No. 5,364,613, U.S. Pat. No. 5,021,556, U.S. Pat. No. 5,075,099, U.S. Pat. No. 5,886,142), and other chelators known in the art including, but not limited to, HYNIC, DTPA, EDTA, DOTA, DO3A, TETA, NOTA and bisamino bisthiol (BAT) chelators (see also U.S. Pat. No. 5,720,934). For example, N.sub.4 chelators are described in U.S. Pat. No. 6,143,274; U.S. Pat. No. 6,093,382; U.S. Pat. No. 5,608,110; U.S. Pat. No. 5,665,329; U.S. Pat. No. 5,656,254; and U.S. Pat. No. 5,688,487. Certain N.sub.35 chelators are described in PCT/CA94/00395, PCT/CA94/00479, PCT/CA95/00249 and in U.S. Pat. No. 5,662,885; U.S. Pat. No. 5,976,495; and U.S. Pat. No. 5,780,006. The chelator also can include derivatives of the chelating ligand mercapto-acetyl-acetyl-glycyl-glycine (MAG3), which contains an N₃S, and N₂S₂ systems such as MAMA (monoamidemonoaminedithiols), DADS (N₂S diaminedithiols), CODADS and the like. These ligand systems and a variety of others are described in, for example, Liu, S, and Edwards, D., 1999. Chem. Rev., 99:2235-2268, and references therein.

The chelator also can include complexes containing ligand atoms that are not donated to the metal in a tetradentate array. These include the boronic acid adducts of technetium and rhenium dioximes, such as are described in U.S. Pat. No. 5,183,653; U.S. Pat. No. 5,387,409; and U.S. Pat. No. 5,118,797, the disclosures of which are incorporated by reference herein, in their entirety.

The chelators can be covalently linked directly to the CD8 capture agent via a linker, as described previously, and then directly labeled with the radioactive metal of choice (see, WO 98/52618, U.S. Pat. No. 5,879,658, and U.S. Pat. No. 5,849,261).

CD8 capture agents comprising ¹⁸F, 4-[¹⁸F]fluorobenzaldehyde (¹⁸FB), Al[¹⁸F]-NOTA, ⁶⁸Ga-DOTA, and ⁶⁸Ga-NOTA are of preferred interest for diagnostic imaging. Complexes of radioactive technetium are also useful for diagnostic imaging, and complexes of radioactive rhenium are particularly useful for radiotherapy. In forming a complex of radioactive technetium with the disclosed reagents, the technetium complex, preferably a salt of ^{99m}Tc pertechnetate, is reacted with the reagent in the presence of a reducing agent. Preferred reducing agents are dithionite, stannous and ferrous ions; the most preferred reducing agent is stannous chloride. Means for preparing such complexes are conveniently provided in a kit form comprising a sealed vial containing a predetermined quantity of a disclosed reagent to be labeled and a sufficient amount of reducing agent to label the reagent with ^{99m}Tc. Alternatively, the complex can be formed by reacting a peptide as disclosed conjugated with an appropriate chelator with a pre-formed labile complex of technetium and another compound known as a transfer ligand. This process is known as ligand exchange and is well known to those skilled in the art. The labile complex can be formed using such transfer ligands as tartrate, citrate, gluconate or mannitol, for example. Among the ⁹⁹mTc pertechnetate salts useful with the disclosed compounds, compositions, and methods are included the alkali metal salts such as the sodium salt, or ammonium salts or lower alkyl ammonium salts.

Preparation of the disclosed complexes where the metal is radioactive rhenium can be accomplished using rhenium starting materials in the +5 or +7 oxidation state. Examples of compounds in which rhenium is in the Re(VII) state are NH₄ReO₄ or KReO₄. Re(V) is available as, for example, [ReOCl₄](NBu₄), [ReOCl₄](AsPh₄), ReOCl₃(PPh₃)₂ and as ReO₂(pyridine)⁴⁺, where Ph is phenyl and Bu is n-butyl. Other rhenium reagents capable of forming a rhenium complex also can be used.

Radioactively labeled PET, SPECT, or scintigraphic imaging agents as disclosed are encompassed having a suitable amount of radioactivity. Generally, the unit dose to be administered has a radioactivity of about 0.01 mCi to about 100 mCi, preferably 1 mCi to 20 mCi. The solution to be injected at unit dosage is from about 0.01 mL to about 10 mL. It is generally preferred to form radioactive complexes in solutions containing radioactivity at concentrations of from about 0.01 mCi to 100 mCi per mL.

Typical doses of a radionuclide-labeled CD8 capture agent provide 10-20 mCi. After injection of the radionuclide-labeled CD8 capture agents into the patient, a gamma camera calibrated for the gamma ray energy of the nuclide incorporated in the imaging agent is used to image areas of uptake of the agent and quantify the amount of radioactivity present in the site. Imaging of the site in vivo can take place in a matter of a few minutes. However, imaging can take place, if desired, in hours or even longer, after the radiolabeled peptide is injected into a patient. In most instances, a sufficient amount of the administered dose will accumulate in the area to be imaged within about 0.1 of an hour to permit the taking of scintiphotos.

Proper dose schedules for the radiotherapeutic compounds as disclosed are known to those skilled in the art. The compounds can be administered using many methods including, but not limited to, a single or multiple IV or IP injections, using a quantity of radioactivity that is sufficient to cause damage or ablation of the targeted CD8-expressing tissue, but not so much that substantive damage is caused to non-target (normal tissue). The quantity and dose required is different for different constructs, depending on the energy and half-life of the isotope used, the degree of uptake and clearance of the agent from the body and the mass of the CD8-expressing tissue. In general, doses can range from a single dose of about 30-50 mCi to a cumulative dose of up to about 3 Ci.

Radiotherapeutic compositions as disclosed can include physiologically acceptable buffers, and can require radiation stabilizers to prevent radiolytic damage to the compound prior to injection. Radiation stabilizers are known to those skilled in the art, and can include, for example, para-aminobenzoic acid, ascorbic acid, gentistic acid and the like.

A single, or multi-vial kit that contains all of the components needed to prepare the disclosed complexes, other than the radionuclide, are specifically contemplated.

A single-vial kit preferably contains a chelating ligand, a source of stannous salt, or other pharmaceutically acceptable reducing agent, and is appropriately buffered with pharmaceutically acceptable acid or base to adjust the pH to a value of about 3 to about 9. The quantity and type of reducing agent used would depend on the nature of the exchange complex to be formed. The proper conditions are well known to those that are skilled in the art. It is preferred that the kit contents be in lyophilized form. Such a single vial kit can optionally contain labile or exchange ligands such as glucoheptonate, gluconate, mannitol, malate, citric or tartaric acid and can also contain reaction modifiers such as diethylenetriamine-pentaacetic acid (DPTA), ethylenediamine tetraacetic acid (EDTA), or alpha, beta, or gamma cyclodextrin that serve to improve the radiochemical purity and stability of the final product. The kit also can contain stabilizers, bulking agents such as mannitol, that are designed to aid in the freeze-drying process, and other additives known to those skilled in the art.

A multi-vial kit preferably contains the same general components but employs more than one vial in reconstituting the radiopharmaceutical. For example, one vial can contain all of the ingredients that are required to form a labile Tc(V) complex on addition of pertechnetate (e.g., the stannous source or other reducing agent). Pertechnetate is added to this vial, and after waiting an appropriate period of time, the contents of this vial are added to a second vial that contains the ligand, as well as buffers appropriate to adjust the pH to its optimal value. After a reaction time of about 5 to 60 minutes, the complexes are formed. It is advantageous that the contents of both vials of this multi-vial kit be lyophilized As above, reaction modifiers, exchange ligands, stabilizers, bulking agents, etc. can be present in either or both vials.

Also provided herein is a method to incorporate an ¹⁸F radiolabeled prosthetic group onto a CD8 capture agent. In some forms, 4-[¹⁸F]fluorobenzaldehyde (¹⁸FB) is conjugated onto a capture agent bearing an aminooxy moiety, resulting in oxime formation. In some forms, [¹⁸F]fluorobenzaldehyde is conjugated onto a capture agent bearing an acyl hydrazide moiety, resulting in a hydrazone adduct. 4-Fluorobenzaldehyde, can be prepared in ¹⁸F form by displacement of a leaving group, using ¹⁸F ion, by known methods.

¹⁸F-labeled capture agents can also be prepared from capture agents possessing thiosemicarbazide moieties under conditions that promote formation of a thiosemicarbozone, or by use of a ¹⁸F-labeled aldehyde bisulfite addition complex.

The above methods are particularly amenable to the labeling of capture agents, e.g., the capture agents described herein, which can be modified during synthesis to contain a nucleophilic hydroxylamine, thiosemicarbazide or hydrazine (or acyl hydrazide) moiety that can be used to react with the labeled aldehyde. The methods can be used for any capture agent that can accommodate a suitable nucleophilic moiety.

Typically the nucleophilic moiety is appended to the N-terminus of the peptide, but the skilled artisan will recognize that the nucleophile also can be linked to an amino acid side chain or to the peptide C-terminus. Methods of synthesizing a radiolabeled peptide sequence are provided in which 4-[¹⁸F]fluorobenzaldehyde is reacted with a peptide sequence comprising either a hydroxylamine, a thiosemicarbazide or a hydrazine (or acyl hydrazide) group, thereby forming the corresponding oximes, thiosemicarbazones or hydrazones, respectively. The 4-[¹⁸F]fluorobenzaldehyde typically is generated in situ by the acid-catalyzed decomposition of the addition complex of 4-[¹⁸F]fluorobenzaldehyde and sodium bisulfite. The use of the bisulfite addition complex enhances the speed of purification since, unlike the aldehyde, the complex can be concentrated to dryness.

Formation of the complex is also reversible under acidic and basic conditions. In particular, when the complex is contacted with a peptide containing a hydroxylamine, a thiosemicarbazide or a hydrazine (or acyl hydrazide) group in acidic medium, the reactive free 4-[¹⁸F]fluorobenzaldehyde is consumed as it is formed in situ, resulting in the corresponding ¹⁸F radiolabeled peptide sequence.

In the instances when the oxime, thiosemicarbazone or hydrazone linkages present in vivo instability, an additional reduction step can be employed to reduce the double bond connecting the peptide to the ¹⁸F bearing substrate. The corresponding reduced peptide linkage would enhance the stability. One of skill in the art would appreciate the variety of methods available to carry out such a reduction step. Reductive amination steps as described in Wilson et al., Journal of Labeled Compounds and Radiopharmaceuticals, XXVIII (10), 1189-1199, 1990 can also be used to form a Schiff's base involving a peptide and 4-[¹⁸F]fluorobenzaldehyde and directly reducing the Schiff's base using reducing agents such as sodium cyanoborohydride.

The 4-[¹⁸F]fluorobenzaldehyde can be prepared as described in Wilson et al., Journal of Labeled Compounds and Radiopharmaceuticals, XXVIII (10), 1189-1199, 1990; Iwata et al., Applied radiation and isotopes, 52, 87-92, 2000; Poethko et al., The Journal of Nuclear Medicine, 45, 892-902, 2004; and Schottelius et al., Clinical Cancer Research, 10, 3593-3606, 2004. The Na¹⁸F in water can be added to a mixture of Kryptofix and K₂CO₃. Anhydrous acetonitrile can be added and the solution is evaporated in a heating block under a stream of argon. Additional portions of acetonitrile can be added and evaporated to completely dry the sample. The 4-trimethylammoniumbenzaldehyde triflate can be dissolved in DMSO and added to the dried F-18. The solution can then be heated in the heating block. The solution can be cooled briefly, diluted with water and filtered through a Waters®. Oasis HLB LP extraction cartridge. The cartridge can be washed with 9:1 water: acetonitrile and water to remove unbound ¹⁸F and unreacted 4-trimethylammoniumbenzaldehyde triflate. The 4-[¹⁸F]fluorobenzaldehyde can then be eluted from the cartridge with methanol in fractions.

Disclosed are methods of using the CD8 capture agents disclosed herein to identify, detect, quantify, and/or separate CD8 in a biological sample. In some forms, these methods utilize an immunoassay, with the capture agent replacing an antibody or its equivalent. In some forms, the immunoassay can be a Western blot, pull-down assay, dot blot, or ELISA.

A biological sample for use in the methods provided herein can be selected from the group consisting of organs, tissue, bodily fluids, and cells. Where the biological sample is a bodily fluid, the fluid can be selected from the group consisting of blood, serum, plasma, urine, sputum, saliva, stool, spinal fluid, cerebral spinal fluid, lymph fluid, skin secretions, respiratory secretions, intestinal secretions, genitourinary tract secretions, tears, and milk. The organs include, e.g., the adrenal glands, bladder, bones, brain, breasts, cervix, esophagus, eyes, gall bladder, genitals, heart, kidneys, large intestine, liver, lungs, lymph nodes, ovaries, pancreas, pituitary gland, prostate, salivary glands, skeletal muscles, skin, small intestine, spinal cord, spleen, stomach, thymus gland, trachea, thyroid, testes, ureters, and urethra. Tissues include, e.g., epithelial, connective, nervous, and muscle tissues.

Disclosed are methods of using the CD8 capture agents disclosed herein to diagnose and/or classify (e.g., stage) a condition associated with CD8 expression. In some forms, the methods comprise (a) obtaining a biological sample from a subject; (b) measuring the presence or absence of CD8 in the sample with the CD8 capture agent; (c) comparing the levels of CD8 to a predetermined control range for CD8; and (d) diagnosing a condition associated with CD8 expression based on the difference between CD8 levels in the biological sample and the predetermined control.

In some forms, the CD8 capture agents disclosed herein are used as a mutant specific targeted therapeutic. In some forms, the CD8 capture agent is administered alone without delivering DNA, a radiopharmaceutical or another active agent.

The CD8 capture agents described herein also can be used to target genetic material to CD8 expressing cells. The genetic material can include nucleic acids, such as RNA or DNA, of either natural or synthetic origin, including recombinant RNA and DNA and antisense RNA and DNA. Types of genetic material that can be used include, for example, genes carried on expression vectors such as plasmids, phagemids, cosmids, yeast artificial chromosomes (YACs) and defective or “helper” viruses, antigene nucleic acids, both single and double stranded RNA and DNA and analogs thereof, such as phosphorothioate and phosphorodithioate oligodeoxynucleotides. Additionally, the genetic material can be combined, for example, with lipids, proteins or other polymers. Delivery vehicles for genetic material can include, for example, a virus particle, a retroviral or other gene therapy vector, a liposome, a complex of lipids (especially cationic lipids) and genetic material, a complex of dextran derivatives and genetic material, etc.

In some forms, the disclosed capture agents can be utilized in gene therapy. In some forms, genetic material, or one or more delivery vehicles containing genetic material can be conjugated to one or more CD8 capture agents of this disclosure and administered to a patient.

Therapeutic agents and the CD8 capture agents disclosed herein can be linked or fused in known ways, optionally using the same type of linkers discussed elsewhere in this application. Preferred linkers will be substituted or unsubstituted alkyl chains, amino acid chains, polyethylene glycol chains, and other simple polymeric linkers known in the art. More preferably, if the therapeutic agent is itself a protein, for which the encoding DNA sequence is known, the therapeutic protein and CD8 binding polypeptide can be coexpressed from the same synthetic gene, created using recombinant DNA techniques, as described above. The coding sequence for the CD8 binding polypeptide can be fused in frame with that of the therapeutic protein, such that the peptide is expressed at the amino- or carboxy-terminus of the therapeutic protein, or at a place between the termini, if it is determined that such placement would not destroy the required biological function of either the therapeutic protein or the CD8 binding polypeptide. A particular advantage of this general approach is that concatamerization of multiple, tandemly arranged CD8 capture agents is possible, thereby increasing the number and concentration of CD8 binding sites associated with each therapeutic protein. In this manner, CD8 binding avidity is increased, which would be expected to improve the efficacy of the recombinant therapeutic fusion protein.

The disclosed compounds, peptides, epitopes, targets, capture agents, and methods can be further understood and modified as described in U.S. Patent Application Publication Nos. 2014/0302998, 2015/0078999, 2015/0219657, 2018/0072772, and 2017/0319722, which are hereby incorporated by reference in the entirety and in particular for their description of compounds, peptides, epitopes, targets, capture agents, and methods.

The disclosed compositions and methods can be further understood through the following numbered paragraphs.

1. A peptide comprising an epitope, wherein the peptide is cross-linked, wherein the epitope corresponds to an epitope of a target, wherein the peptide does not include the entire target, wherein the cross-link is a disulfide, preferably a disulfide that naturally occurs in the target or between added or substitute cysteines.

2. The peptide of paragraph 1, wherein the target is a target protein.

3. The peptide of paragraph 2, wherein the target protein is CD8.

4. The peptide of any one of paragraphs 1-3, wherein the epitope comprises amino acids 24 to 33 of CD8.

5. The peptide of any one of paragraphs 1-4, wherein the disulfide is between amino acids C22 and C33 of CD8.

6. The peptide of any one of paragraphs 1-5, wherein the peptide comprises amino acids 21 to 35 of CD8.

7. The peptide of any one of paragraphs 1-6 further comprising an alkyne moiety, an azide moiety, a reporter moiety, or combinations thereof.

8. The peptide of paragraph 7, wherein the azide moiety and the alkyne moiety are comprised in an artificial amino acid.

9. The peptide of paragraph 8, wherein the artificial amino acid is propargylglycine (Fra).

9A. The peptide of paragraph 8, wherein the artificial amino acid replaces L26 of CD8.

10. The peptide of any one of paragraphs 7-9A, wherein the reporter moiety is biotin.

11. The peptide of any one of paragraphs 1-3 further comprising an alkyne moiety, an azide moiety, a reporter moiety, or combinations thereof, wherein the epitope comprises a phosphorylated amino acid, wherein the azide moiety and the alkyne moiety of the target peptide are metalorganic molecules that selectively bind to the phospho group on the phosphorylated amino acid.

12. The peptide of paragraph 11, wherein the target is a target protein, wherein the target protein is CD8, wherein the phosphorylated amino acid is Ser235 or Ser236 of CD8.

13. The peptide of paragraph 11 or 12, wherein the metalorganic molecule comprises the reporter moiety.

14. The peptide of paragraph 13, wherein the reporter moiety is biotin.

15. A method of preparing a peptide comprising an epitope, wherein the epitope corresponds to an epitope of a target, wherein the peptide does not include the entire target, the method comprising cross-linking the peptide, wherein the cross-link is a disulfide, preferably a disulfide that naturally occurs in the target or between added or substitute cysteines.

16. The method of paragraph 15, wherein the target is a target protein.

17. The method of paragraph 16, wherein the target protein is CD8.

18. The method of any one of paragraphs 15-17, wherein the epitope comprises amino acids 24 to 33 of CD8.

19. The method of any one of paragraphs 15-18, wherein the disulfide is between amino acids C22 and C33 of CD8.

20. The method of any one of paragraphs 15-19, wherein the peptide comprises amino acids 21 to 35 of CD8.

21. The method of any one of paragraphs 15-20, wherein the peptide further comprises an alkyne moiety, an azide moiety, a reporter moiety, or combinations thereof.

22. The method of paragraph 21, wherein the azide moiety and the alkyne moiety are comprised in an artificial amino acid.

23. The method of paragraph 22, wherein the artificial amino acid is propargylglycine (Fra).

24. The method of any one of paragraphs 21-23, wherein the reporter moiety is biotin.

25. The method of any one of paragraphs 15-20, wherein the peptide further comprises an alkyne moiety, an azide moiety, a reporter moiety, or combinations thereof, wherein the epitope comprises a phosphorylated amino acid, wherein the azide moiety and the alkyne moiety of the target peptide are metalorganic molecules that selectively bind to the phospho group on the phosphorylated amino acid.

26. The method of paragraph 25, wherein the target is a target protein, wherein the target protein is CD8, wherein the phosphorylated amino acid is Ser235 or Ser236 of CD8.

27. The method of paragraph 25 or 26, wherein the metalorganic molecule comprises the reporter moiety.

28. The method of paragraph 27, wherein the reporter moiety is biotin.

29. A method for identifying a target binding compound, the method comprising:

(A) contacting a first peptide library with a target peptide comprising an epitope,

wherein the first peptide library comprising a plurality of first peptide library members, wherein the first peptide library members optionally individually comprise an alkyne, azide, reporter moiety, or combinations thereof,

wherein the target peptide is cross-linked, wherein the epitope corresponds to an epitope of a target, wherein the target peptide does not include the entire target, wherein the cross-link is a disulfide, preferably a disulfide that naturally occurs in the target or between added or substitute cysteines, wherein the target peptide optionally comprises an alkyne moiety, an azide moiety, a reporter moiety, or combinations thereof;

(B) identifying a first peptide library member with affinity for a first binding site on the epitope; and optionally:

(C) contacting a second peptide library with a composition comprising (i) the target or the target peptide and (ii) the first peptide library member of step B, wherein, prior to contacting, the first peptide library member of step B is modified to include an alkyne moiety or an azide moiety,

wherein the second peptide library comprises a plurality of second peptide library members, wherein the second peptide library members each comprise an azide moiety, an alkyne moiety, or both,

whereby a triazole-linked conjugate is formed between the first peptide library member of step B and a second peptide library member, whereby the triazole-linked second peptide library member is identified as having affinity for a second binding site on the target or the target peptide.

30. The method of paragraph 29, wherein the epitope is a distinct molecular surface of the target.

31. The method of paragraph 29, wherein the target peptide provides a catalytic scaffold for promoting the covalent coupling of the azide moiety and the alkyne moiety to form the triazole linkage.

32. The method of paragraph 29, wherein the azide moiety and the alkyne moiety of the first peptide library member of step B are comprised in an artificial amino acid.

33. The method of paragraph 32, wherein the artificial amino acid is propargylglycine (Fra).

33A. The method of paragraph 32, wherein the artificial amino acid replaces L26 of CD8.

34. The method of paragraph 29, wherein the epitope comprises a phosphorylated amino acid, wherein the azide moiety and the alkyne moiety of the target peptide are metalorganic molecules that selectively bind to the phospho group on the phosphorylated amino acid.

35. The method of paragraph 34, wherein the metalorganic molecule comprises the reporter moiety.

36. The method of paragraph 35, wherein the reporter moiety is biotin.

37. The method of paragraph 34, wherein the metalorganic molecule comprises an azide moiety.

38. The method of paragraph 29, wherein the reporter moiety is biotin.

39. The method of paragraph 29 further comprising:

(C) contacting a second peptide library with a composition comprising (i) the target or the target peptide and (ii) the first peptide library member of step B, wherein, prior to contacting, the first peptide library member of step B is modified to include an alkyne moiety or an azide moiety,

wherein the second peptide library comprises a plurality of second peptide library members, wherein the second peptide library members each comprise an azide moiety, an alkyne moiety, or both,

whereby a triazole-linked conjugate is formed between the first peptide library member of step B and a second peptide library member, whereby the triazole-linked second peptide library member is identified as having affinity for a second binding site on the target or the target peptide.

40. The method of paragraph 39 further comprising:

(D) contacting a third peptide library with a composition comprising (i) the target or the target peptide and (ii) the triazole-linked conjugate formed in step C, wherein, prior to contacting, the triazole-linked conjugate formed in step C is modified to include an alkyne moiety or an azide moiety,

wherein the third peptide library comprises a plurality of third peptide library members, wherein the third peptide library members each comprise an azide moiety, an alkyne moiety, or both,

whereby a triazole-linked conjugate is formed between the triazole-linked conjugate formed in step C and a third peptide library member, whereby the triazole-linked third peptide library member is identified as having affinity for a third binding site on the target or the target peptide.

41. The method of paragraph 40 further comprising:

(E) contacting a fourth peptide library with a composition comprising (i) the target or the target peptide and (ii) the triazole-linked conjugate formed in step D, wherein, prior to contacting, the triazole-linked conjugate formed in step D is modified to include an alkyne moiety or an azide moiety,

wherein the fourth peptide library comprises a plurality of fourth peptide library members, wherein the fourth peptide library members each comprise an azide moiety, an alkyne moiety, or both,

whereby a triazole-linked conjugate is formed between the triazole-linked conjugate formed in step D and a fourth peptide library member, whereby the triazole-linked fourth peptide library member is identified as having affinity for a fourth binding site on the target or the target peptide.

42. The method of paragraph 41 further comprising:

(F) contacting an nth peptide library with a composition comprising (i) the target or the target peptide and (ii) the triazole-linked conjugate formed in the immediately prior contacting step, wherein, prior to contacting, the triazole-linked conjugate formed in he immediately prior contacting step is modified to include an alkyne moiety or an azide moiety,

wherein the nth peptide library comprises a plurality of nth peptide library members, wherein the nth peptide library members each comprise an azide moiety, an alkyne moiety, or both,

whereby a triazole-linked conjugate is formed between the triazole-linked conjugate formed in the immediately prior contacting step and an nth peptide library member, whereby the triazole-linked nth peptide library member is identified as having affinity for an nth binding site on the target or the target peptide.

43. The method of paragraph 42 further comprising repeating step F one or more times.

44. The method of any one of paragraphs 29-43, wherein the first peptide library member of step B is identified by selecting a first peptide library member linked to the target peptide via a triazole linkage.

45. The method of any one of paragraphs 39-44, wherein the second peptide library member of step C is identified by selecting a second peptide library member linked to the first peptide library member via a triazole linkage.

46. The method of any one of paragraphs 40-45, wherein the third peptide library member of step D is identified by selecting a third peptide library member linked to the triazole-linked conjugate formed in step C via a triazole linkage.

47. The method of any one of paragraphs 41-46, wherein the fourth peptide library member of step E is identified by selecting a fourth peptide library member linked to the triazole-linked conjugate formed in step D via a triazole linkage.

48. The method of any one of paragraphs 42-47, wherein the nth peptide library member of step F is identified by selecting an nth peptide library member linked to the triazole-linked conjugate formed in the immediately prior contacting step via a triazole linkage.

49. The method of any one of paragraphs 44-48, wherein one or more or the triazole-linked peptide library members are selected by selecting a peptide library member labeled with the reporter moiety.

50. The method of any one of paragraphs 29-49, wherein the target is a target protein, wherein the method further comprises testing the first peptide library member of step B for binding to the target protein.

51. The method of any one of paragraphs 39-50, wherein the target is a target protein, wherein the method further comprises testing the triazole-linked conjugate formed in step C for binding to the target protein.

52. The method of any one of paragraphs 40-51, wherein the target is a target protein, wherein the method further comprises testing the triazole-linked conjugate formed in step D for binding to the target protein.

53. The method of any one of paragraphs 41-52, wherein the target is a target protein, wherein the method further comprises testing the triazole-linked conjugate formed in step E for binding to the target protein.

54. The method of any one of paragraphs 42-53, wherein the target is a target protein, wherein the method further comprises testing the triazole-linked conjugate formed in the immediately prior contacting step for binding to the target protein.

55. The method of any one of paragraphs 39-54, wherein the second peptide library is contacted with a composition comprising the target peptide and the first peptide library member of step B.

56. The method of any one of paragraphs 39-54, wherein the second peptide library is contacted with a composition comprising the target and the first peptide library member of step B.

57. The method of any one of paragraphs 40-56, wherein the third peptide library is contacted with a composition comprising the target peptide and the triazole-linked conjugate formed in step C.

58. The method of any one of paragraphs 40-56, wherein the third peptide library is contacted with a composition comprising the target and the triazole-linked conjugate formed in step C.

59. The method of any one of paragraphs 41-58, wherein the fourth peptide library is contacted with a composition comprising the target peptide and the triazole-linked conjugate formed in step D.

60. The method of any one of paragraphs 41-58, wherein the fourth peptide library is contacted with a composition comprising the target and the triazole-linked conjugate formed in step D.

61. The method of any one of paragraphs 42-60, wherein the nth peptide library is contacted with a composition comprising the target peptide and the triazole-linked conjugate formed in the immediately prior contacting step.

62. The method of any one of paragraphs 42-60, wherein the nth peptide library is contacted with a composition comprising the target and the triazole-linked conjugate formed in the immediately prior contacting step.

63. The method of any one of paragraph 29-62, wherein the epitope is a distinct molecular surface of the target.

64. The method of any one of paragraphs 29-63, further comprising determining the peptide sequence of the first peptide library member of step B.

65. The method of any one of paragraphs 39-64, further comprising determining the peptide sequence of the second peptide library member of step C.

66. The method of any one of paragraphs 40-65, further comprising determining the peptide sequence of the third peptide library member of step D.

67. The method of any one of paragraphs 41-66, further comprising determining the peptide sequence of the fourth peptide library member of step E.

68. The method of any one of paragraphs 42-67, further comprising determining the peptide sequence of the nth peptide library member of step F.

69. The method of any one of paragraphs 64-68, wherein the peptide sequence of one or more of the peptide library members is determined by Edman degradation.

70. The method of any one of paragraphs 29-39, further comprising modifying the triazole linked conjugate formed in step C to contain a triazole or alkyne and contacting the modified conjugate with the target or the target peptide and a third peptide library, the third peptide library comprising a plurality of third peptide library members, each third peptide library member comprising an azide or alkyne, thereby forming a triazole linkage between the modified conjugate and a member of the third peptide library, wherein the third peptide library member is identified as having affinity for a third binding site on the target or the target peptide.

71. The method of paragraph 70, further comprising modifying the triazole linked conjugate formed between the modified conjugate and the member of the third peptide library to contain a triazole or alkyne and contacting the modified conjugate with the target or the target peptide and a fourth peptide library, the fourth peptide library comprising a plurality of fourth peptide library members, each fourth peptide library member comprising an azide or alkyne, thereby forming a triazole linkage between the modified conjugate and a member of the fourth peptide library, wherein the fourth peptide library member is identified as having affinity for a fourth binding site on the target or the target peptide.

72. The method of paragraph 71, further comprising modifying the triazole linked conjugate formed between the modified conjugate and the identified fourth peptide library member to contain a triazole or alkyne and contacting the modified conjugate with the target or the target peptide and an nth peptide library, the nth peptide library comprising a plurality of nth peptide library members, each nth peptide library member comprising an azide or alkyne, thereby forming a triazole linkage between the modified conjugate and a member of the nth peptide library, wherein the nth peptide library member is identified as having affinity for an nth binding site on the target or the target peptide.

73. The method of paragraph 72, further comprising:

(i) modifying the triazole linked conjugate formed between the modified conjugate and the identified nth peptide library member to contain a triazole or alkyne and

(ii) contacting the modified conjugate with the target or the target peptide and an N+1th peptide library, the N+1th peptide library comprising a plurality of N+1th peptide library members, each n+1th peptide library member comprising an azide or alkyne, thereby forming a triazole linkage between the modified conjugate and a member of the n+1th peptide library, the n+1th peptide library member having affinity for an n+1th binding site on the target or the target peptide,

optionally repeating steps (i) and (ii) one or more times.

74. The method of any one of paragraphs 29-73, wherein the first binding site is an epitope.

75. The method of any one of paragraphs 29-74, wherein the second binding site is a second epitope.

76. The method of any one of paragraphs 40-75, wherein the third binding site is a third epitope.

77. The method of any one of paragraphs 40-75, wherein the fourth binding site is a fourth epitope.

78. The method of any one of paragraphs 29-77, wherein the target is a protein.

79. The method of paragraph 78, wherein the protein is an enzyme or cell surface protein.

80. The method of paragraph 79, wherein the protein is CD8.

81. The peptide of any one of paragraphs 1-14 or the method of any one of paragraphs 15-80, wherein the target peptide comprises a linkage to a reporter moiety, wherein the reporter moiety comprises polyethylene glycol (PEG), biotin, thiol, a fluorophore, or combinations thereof.

82. The peptide or method of paragraph 81, wherein the fluorophore is carboxyfluorescein (FAM), fluorescein isothiocyanate (FITC), Cyanine-5 (Cy5), tetramethylrhodamine (TRITC) or Carboxytetramethylrhodamine (TAMRA).

83. A capture agent for CD8, the capture agent comprising a first ligand that specifically binds to a first epitope of CD8.

84. The capture agent of paragraph 83 further comprising a second ligand, wherein the first ligand and the second ligand are covalently linked to each other, wherein the second ligand specifically binds to a second epitope of CD8 that is distinct from the epitope to which the first ligand specifically binds.

85. The capture agent of paragraph 84, wherein the first and second epitopes are in different locations on CD8.

86. The capture agent of paragraph 84 or 85 further comprising a linker covalently connecting the first ligand to the second ligand.

87. The capture agent of any one of paragraphs 84-86, wherein the first ligand comprises an amino acid sequence 80-100% identical to an amino acid sequence selected from the group consisting of tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), and khyta (SEQ ID NO:89).

88. The capture agent of any one of paragraphs 84-86, wherein the first ligand comprises an amino acid sequence 80-100% identical to an amino acid sequence selected from the group consisting of hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), and teGwf (SEQ ID NO:20).

89. The capture agent of any one of paragraphs 84-86, wherein the first ligand comprises an amino acid sequence 80-100% identical to an amino acid sequence selected from the group consisting of krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), and hGrGh (SEQ ID NO:74).

90. The capture agent of any one of paragraphs 84-86, wherein the first ligand comprises an amino acid sequence 80-100% identical to an amino acid sequence selected from the group consisting of tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), khyta (SEQ ID NO:89), hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), teGwf (SEQ ID NO:20), krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), hGrGh (SEQ ID NO:74), nsprw (SEQ ID NO:83), and herlk (SEQ ID NO:84).

91. The capture agent of any one of paragraphs 84-86, wherein the first ligand comprises an amino acid sequence selected from the group consisting of tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), and khyta (SEQ ID NO:89).

92. The capture agent of any one of paragraphs 84-86, wherein the first ligand comprises an amino acid sequence selected from the group consisting of hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), and teGwf (SEQ ID NO:20).

93. The capture agent of any one of paragraphs 84-86, wherein the first ligand comprises an amino acid sequence selected from the group consisting of krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), and hGrGh (SEQ ID NO:74).

94. The capture agent of any one of paragraphs 84-86, wherein the first ligand comprises an amino acid sequence selected from the group consisting of tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), khyta (SEQ ID NO:89), hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), teGwf (SEQ ID NO:20), krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), hGrGh (SEQ ID NO:74), nsprw (SEQ ID NO:83), and herlk (SEQ ID NO:84).

95. The method of any one of paragraphs 15-82 or the capture agent of any one of paragraphs 83-86, wherein the epitope is 5 to 30 amino acids long, preferably the epitope is 8 to 20 amino acids long, more preferably the epitope is 7 to 13 amino acids long.

96. The method of any one of paragraphs 15-82 or the capture agent of any one of paragraphs 83-86, wherein the first epitope is, at most, 5, 6, 7, 8, 9, 10, 11, 12, 13, 14, 15, 16, 17, 18, 19 or 20 amino acids long.

97. The method of any one of paragraphs 15-82 or the capture agent of any one of paragraphs 83-86, wherein the first epitope comprises the amino acid sequence KCQVLLSNPTSGCSW (SEQ ID NO:1).

98. The method of paragraph 97, wherein L26 is replaced with an azide-containing amino acid residue.

99. The method of any one of paragraphs 15-82 or the capture agent of any one of paragraphs 83-86, wherein the first epitope comprises the amino acid sequence SQFRVSPLDRTWNLGETVELKSQVL (SEQ ID NO:97).

100. The method of paragraph 99, wherein N13 is replaced with an azide-containing amino acid residue.

101. The method of any one of paragraphs 15-82 or the capture agent of any one of paragraphs 83-86, wherein the first epitope comprises the amino acid sequence SQFRVSPLDR (SEQ ID NO:2).

102. The method of paragraph 101, wherein an azide-containing amino acid residue is added ahead of S1.

103. The method of any one of paragraphs 15-82 or the capture agent of any one of paragraphs 83-86, wherein the first epitope comprises the amino acid sequence VLLSNPTSGC (SEQ ID NO:3).

104. The method of paragraph 103, wherein an azide-containing amino acid residue is added ahead of V24.

105. The method of any one of paragraphs 15-82 or the capture agent of any one of paragraphs 83-86, wherein the first epitope comprises the amino acid sequence KAAEGLDTQRFSGKRLGDTF (SEQ ID NO:4).

106. The method of paragraph 105, wherein R67 is replaced with an azide-containing amino acid residue.

107. The method of any one of paragraphs 15-82 or the capture agent of any one of paragraphs 83-86, wherein the first epitope comprises the amino acid sequence FSGKRLGDTFVLTLSD (SEQ ID NO:5).

108. The method of paragraph 107, wherein G74 is replaced with an azide-containing amino acid residue.

109. The method of any one of paragraphs 15-82 or the capture agent of any one of paragraphs 83-86, wherein the first epitope comprises the amino acid sequence SALSNSIMYFSHFVPV (SEQ ID NO:6).

110. The method of paragraph 109, wherein M102 is replaced with an azide-containing amino acid residue.

111. The method of any one of paragraphs 15-82 or the capture agent of any one of paragraphs 83-86, wherein the first epitope comprises the amino acid sequence RFSGKRLGDTFVLTLSD (SEQ ID NO:98).

112. The method of paragraph 111, wherein G74 is replaced with an azide-containing amino acid residue.

113. The method of any one of paragraphs 15-82 or the capture agent of any one of paragraphs 83-86, wherein the first epitope comprises the amino acid sequence QNKPKAAEGLDTQRF (SEQ ID NO:99).

114. The method of paragraph 113, wherein L63 is replaced with an azide-containing amino acid residue.

115. The method of any one of paragraphs 15-82 or the capture agent of any one of paragraphs 83-86, wherein the first epitope comprises the amino acid sequence FQPRGAAASPTFL (SEQ ID NO:7).

116. The method of any one of paragraphs 15-82 or the capture agent of any one of paragraphs 83-86, wherein the first epitope comprises the amino acid sequence LYLSQNKPKAA (SEQ ID NO:8).

### EXAMPLES

### Example 1

### Cross-Linked Synthetic Epitope of CD8

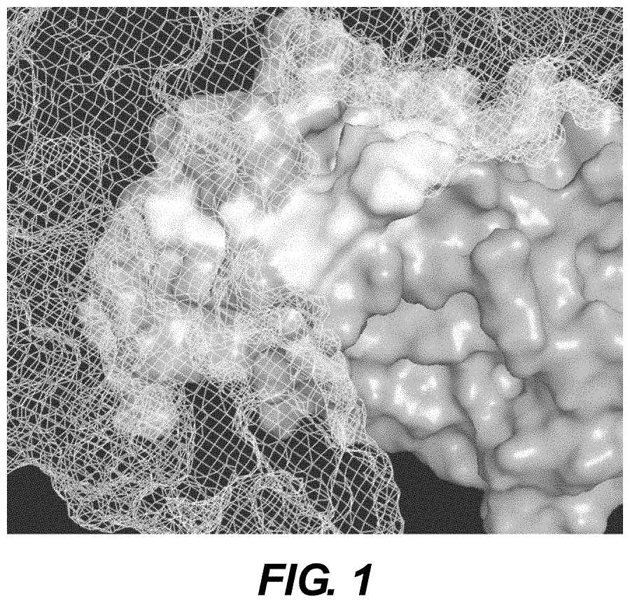

A modified form of epitope B of CD8 was developed. Epitope B is V24 to C33 of CD8 (VLLSNPTSGC; SEQ ID NO:3). The new epitope, epitope B2, increased the length of epitope B to include the cysteines involved in a disulfide in CD8. Epitope B2 is K21 to W35 of CD8 (KCQVLLSNPTSGCSW; SEQ ID NO:1). The cysteines are bolded; the amino acid replaced with Az4 (L26) is in bold italic. FIG. 1 shows epitope B2 (white space filling structure) in an equilibrated CD8 homodimer (grey space filling structure) interacting with an equilibrated MHC domain (wire structure). The solvent accessible surface area is 601.524 Å². The total surface area is 1658.398 Å². The net charge is +1. The average equilibrated backbone RMSD is 1.284 (0.197). The average equilibrated total RMSD is 1.476 (0.206).

Constrained epitope B2 was used as the target in an epitope targeting screen against a peptide library. The constrained epitope B2 had L26 replaced with an azide-containing residue (Az4). The library peptides included alkyne groups. Binding of library peptides in proximity to and in the correct orientation to the Az4 residue on epitope B2 results in reaction of the azide on the Az4 residue with the alkyne on the bound library peptide to form a triazole linkage between epitope B2 and the bound library peptide. Formation of this linkage defines the hit library peptides. The hit peptides are hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), and teGwf (SEQ ID NO:20).

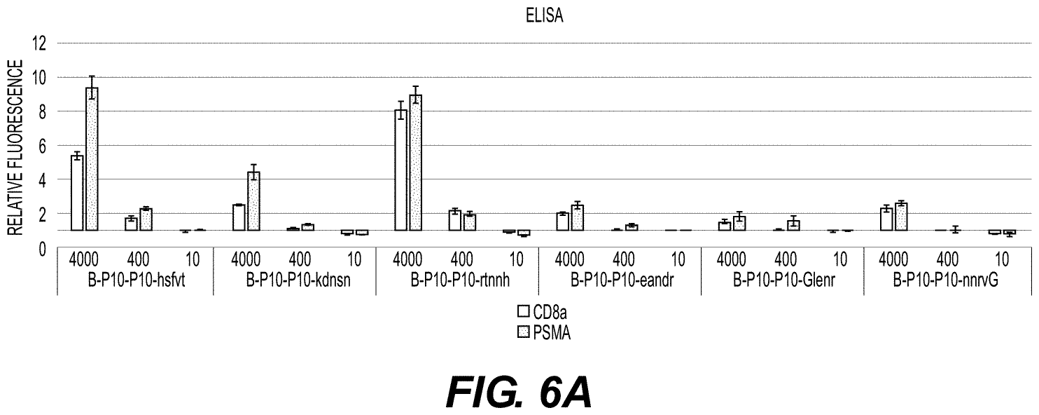

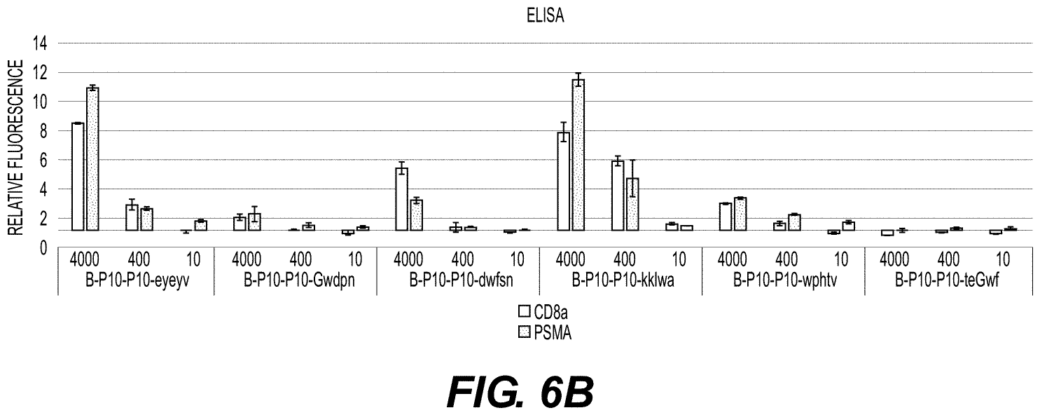

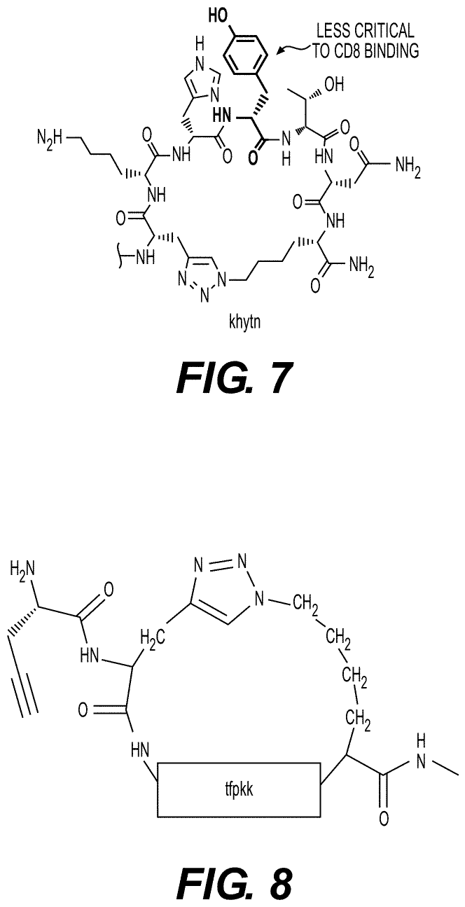

Hits from the constrained epitope B2 screen were tested for binding to CD8a or non-specific binding to PSMA by ELISA using three different PCC concentrations (4000 nM, 400 nM, 10 nM) (FIG. 6). These results were compared with previously obtained flow cytometry data against CD8+and CD8- cells (data note shown). ELISA results were consistent with flow data. Most of the hits show either non-specific binding or no binding.

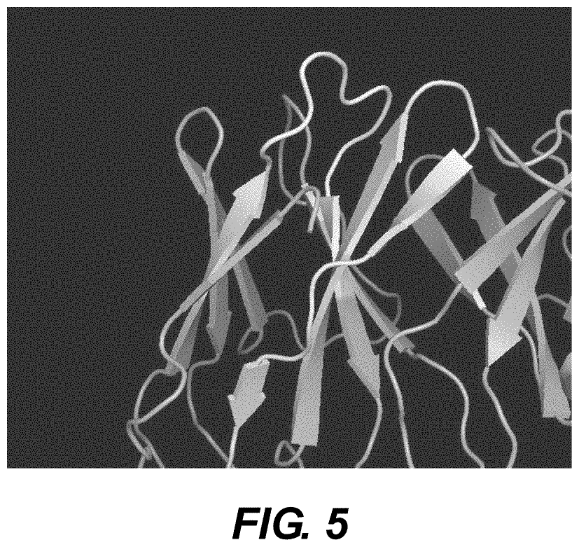

Other new epitopes of CD8 were developed. These include epitope A (SQFRVSPLDR (51-R10); SEQ ID NO:2; FIG. 4A), epitope B (VLLSNPTSGC (V24-C33); SEQ ID NO:3; FIG. 4B), epitope C (KAAEGLDTQRFSGKRLGDTF (K58-F77); SEQ ID NO:4; epitope D (FSGKRLGDTFVLTLSD (F68-D83)SEQ ID NO:5; epitope E (SALSNSIMYFSHFVPV (S95-V110); SEQ ID NO:6; FIG. 5), epitope F (FQPRGAAASPTFL (F37-L49); SEQ ID NO:7; FIG. 4C), and epitope G (LYLSQNKPKAA (L50-A60); SEQ ID NO:8; FIG. 4D). The amino acid replaced with Az4 for screening in epitope C (R67), epitope D (G74), and epitope E (M102) is in bold italic (Az4 was added at the N-terminal end of epitopes A and B). For screening, the epitope peptides also had biotin and PEGS added at the N-terminal end. The solvent accessible surface area of epitope E is 975.559 Å². The total surface area of epitope E is 1880.344 Å². The net charge is 0.

Hit peptide against epitope B2 Gwdpn was chosen for use as a ligand in a biligand with a ligand for epitope E. Epitopes B2 and E are two loops that are adjacent to each other on the CD8 surface (FIG. 5). The amino acids in CD8 that correspond to the amino acids in epitopes B2 and E replaced with Az4 residues for screening are approximately 12 Angstroms away from each other. A PEG3 should span the between the epitope B2 ligand and the epitope E ligand.

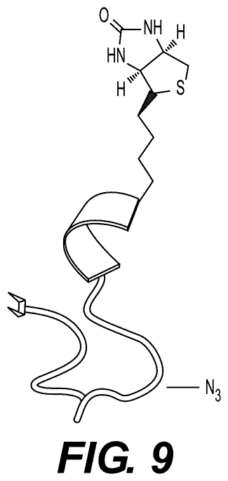

The epitope E ligand used in the biligand is tfpkk (SEQ ID NO:21). It was determined that tfpkk (SEQ ID NO:21) binds to epitope E by screening an alkyne-containing tfpkk peptide (SEQ ID NO:21) coupled to a bead (FIG. 8) with azide- and biotin-containing epitopes A, B, C, D, and E (FIG. 9). The epitopes used in the screen were: Epitope A (a.a. 1-10): Biotin-PEG₃-[Az4]-SQFRVSPLDR (SEQ ID NO:22); Epitope B (a.a. 24-32): Biotin-PEG₃-[Az4]-VLLSNPTSG (SEQ ID NO:23); Epitope C (a.a. 58-77): Biotin-PEG₃-KAAEGLDTQ-[Az4]-FSGKRLGDTF (SEQ ID NO:24); Epitope D (a.a. 68-83): Biotin-PEG₃-FSGKRL-[Az4]-DTFVLTLSD (SEQ ID NO:25); Epitope E (a.a. 95-110): Biotin-PEG₃-SALSNSI-[Az4]-YFSHFVPV (SEQ ID NO:26).

Development of the beads with alkaline phosphatase-linked streptavidin produced color only when epitope E was used. This is in agreement with docking calculations of binding affinities of the epitopes to tfpkk (SEQ ID NO:21) and a control peptide (krsah; SEQ ID NO:27) (Table 2).

TABLE 2
Binding Affinities of CD8 Epitopes with Peptides
Calculated from Docking Analysis

| Sequence | | | | | Binding Affinities G (kcal/mol) | | | | |
|---|---|---|---|---|---|---|---|---|---|
| xl | x2 | x3 | x4 | x5 | EpiA | EpiB | EpiC | EpiD | EpiE |
| k | r | s | a | h | −7.8 | −6.2 | −5.7 | −7.5 | −8.9 |
| t | f | p | k | k | −508 | −6.1 | −5.2 | −5.7 | −7.3 |

The epitope B2 and E biligands use the ligands tfpkk and Gwdpn. To make the most effective biligand, are two biligands using a PEG3 linker are being produced, reversing tfpkk (SEQ ID NO:21) and Gwdpn (SEQ ID NO:16) orientation in the two biligands. Alanine scanning indicated that the phenylalanine and first lysine residues of are less important (i.e., not critical) to binding of the tfpkk ligand to the target. Thus, additional ligands for the B2 epitope include tx₁px₂k, where x₁ and x₂ are, independently, any amino acid, any amino acid other than Cys, any D-amino acid, or any D-amino acid other than Cys.

### Example 2

### CD8 Ligands

CD8 ligands tfpkk (SEQ ID NO:21), krsah (SEQ ID NO:27), and snprk (SEQ ID NO:28) were discovered by screening a peptide library with a cocktail of five CD8α epitopes, screening the resultant hits for binding to human CD8αα, screening the resultant hits for binding to CD8+Sup T1 cells, and, finally, confirming that the resultant hits do not bind CD8-Jurkat cells. Three hits were left after this screening (Table 3). These hits were assessed in solid phase ELISA and in flow cytometry experiments.

TABLE 3
Hit peptides in five epitope cocktail screening

| x1 | x2 | x3 | x4 | x5 |
|---|---|---|---|---|
| k | r | s | a | h |
| t | f | p | k | k |
| s | n | p | r | k |

With the 1kPEG linker, binding of tfpkk (SEQ ID NO:21) to CD8+SupT1 cells was observed by flow cytometry, while little binding was seen in CD8−Jurkat cells. Binding of krsah (SEQ ID NO:27) was detected on both CD8+SupT1 cells and CD8−Jurkat cells. The mean fluorescence intensity ratio to each of the ligands tfpkk (SEQ ID NO:21), krsah (SEQ ID NO:27), and snprk (SEQ ID NO:28) was determined (Table 4). The mean fluorescence intensity ratio of different amounts of the ligand tfpkk (SEQ ID NO:21) was determined (Table 5).

TABLE 4
Mean Fluorescence Intensity Ratio of hit CD8 peptides

| | SupT1 | Jurkat |
|---|---|---|
| | MFIR | MFIR |
| unstained | 1 | 1 |
| Biotin anti-CD8a Ab (Clone RPA-T8) | 73.9 | 1.0 |
| Biotin-1kPEG | 1.0 | 1.0 |
| Biotin-1kPEG-cy(krsah) (SEQ ID NO: 27) | 1.6 | 1.3 |
| Biotin-1kPEG-cy(snprk) (SEQ ID NO: 28) | 1.1 | 1.0 |
| Biotin-1kPEG-cy(tfpkk) (SEQ ID NO: 21) | 1.8 | 1.2 |

MFIR = Mean Fluorescence Intensity Ratio (relative to unstained)

TABLE 5
Mean Fluorescence Intensity Ratio of tfpkk ligand
(SEQ ID NO: 21) at different concentrations

| | SupT1 | Jurkat |
|---|---|---|
| | MFIR | MFIR |
| unstained | 1 | 1 |
| Biotin anti-CD8a Ab (Clone RPA-T8) | 86.6 | 1.0 |
| Biotin-1k PEG-tfpkk 4 μM | 1.5 | 1.1 |
| Biotin-1k PEG-tfpkk 1.3 μM | 1.1 | 1.0 |
| Biotin-1k PEG-tfpkk 0.4 μM | 1.1 | 1.0 |
| Biotin-1k PEG-tfpkk 0.15 μM | 1.0 | 1.0 |
| Biotin-1k PEG-tfpkk 0.05 μM | 1.0 | 1.0 |

MFIR = Mean Fluorescence Intensity Ratio (relative to unstained)

The binding of Biotin-1kPEG-tfpkk was observed by flow cytometry at 4 mM, but little to no binding was detected at lower concentrations.

Binding of Biotin-1kPEG-tfpkk (SEQ ID NO:21) to CD8+SupT1 cells or CD8−Jurkat was performed at room temperature (RT) or 4° C. Increased temperature does not change the binding of Biotin-1kPEG-tfpkk (SEQ ID NO:21) to CD8+SupT1 cells, but slightly increases non-specific binding to CD8−Jurkat cells (Table 6).

TABLE 6
Mean Fluorescence Intensity Ratio of tfpkk ligand
(SEQ ID NO: 21) at different temperatures

| | SupT1 | Jurkat |
|---|---|---|
| | MFIR | MFIR |
| unstained | 1 | 1 |
| Biotin-1k PEG-tfpkk 4 μM RT | 1.5 | 1.3 |
| Biotin-1k PEG-tfpkk 4 μM 4° C. | 1.5 | 1.1 |

MFIR = Mean Fluorescence Intensity Ratio (relative to unstained)

With the 1kPEG linker, binding of tfpkk (SEQ ID NO:21) to CD8+SupT1 cells was observed by flow cytometry, while little binding was seen in CD8−Jurkat cells (Table 7). Binding of krsah (SEQ ID NO:27) can be detected on CD8+SupT1 cells, but binding to CD8−Jurkat cells is also observed (Table 7).

TABLE 7
Mean Fluorescence Intensity Ratio of CD8 epitope ligands

| | SupT1 | Jurkat |
|---|---|---|
| | MFIR | MFIR |
| unstained | 1 | 1 |
| Biotin anti-CD8a Ab (Clone RPA-T8) | 73.9 | 1.0 |
| Biotin-1kPEG | 1.0 | 1.0 |
| Biotin-1kPEG-cy(krsah) (SEQ ID NO: 27) | 1.6 | 1.3 |
| Biotin-1kPEG-cy(snprk) (SEQ ID NO: 28) | 1.1 | 1.0 |
| Biotin-1kPEG-cy(tfpkk) (SEQ ID NO: 21) | 1.8 | 1.2 |

MFIR = Mean Fluorescence Intensity Ratio (relative to unstained)

CD8 ligands nfpkk (SEQ ID NO:29) and vlyrr (SEQ ID NO:30) were discovered by screening a peptide library with human CD8α/β, screening the resultant hits for binding to CD8+Sup T1 cells, and, finally, confirming that the resultant hits do not bind CD8−Jurkat cells. Eight hits were left after this screening (Table 8) (from top to bottom: SEQ ID NOs:31, 32, 33, 34, 30, 35, 36, 37). These hits were assessed in solid phase ELISA and in flow cytometry experiments (Table 9).

TABLE 8
Hit peptides in human CD8α/β screening

| x1 | x2 | x3 | x4 | x5 | |
|---|---|---|---|---|---|
| y | r | p | f | y | SEQ ID NO: 31 |
| n | f | y | r | r | SEQ ID NO: 32 |
| y | f | r | s | r | SEQ ID NO: 33 |
| y | r | s | n | y | SEQ ID NO: 34 |
| v | l | y | r | r | SEQ ID NO: 30 |
| r | p | y | a | y | SEQ ID NO: 35 |
| a | y | k | f | n | SEQ ID NO: 36 |
| r | f | t | a | f | SEQ ID NO: 37 |

TABLE 9
Mean Fluorescence Intensity Ratio of hit CD8 peptides

| | SupT1 | Jurkat |
|---|---|---|
| | MFIR | MFIR |
| Biotin-1kPEG | 1 | 1 |
| Biotin-1kPEG-yrpfy (SEQ ID NO: 31) | 1.1 | 1.0 |
| Biotin-1kPEG-nfyrr (SEQ ID NO: 32) | 1.2 | 1.1 |
| Biotin-1kPEG-yfrsr (SEQ ID NO: 33) | 1.1 | 1.1 |
| Biotin-1kPEG-yrsny (SEQ ID NO: 34) | 1.0 | 1.0 |
| Biotin-1kPEG-vlyrr (SEQ ID NO: 30) | 2.0 | 2.0 |
| Biotin-1kPEG-rpyay (SEQ ID NO: 35) | 1.0 | 1.0 |
| Biotin-1kPEG-aykfn (SEQ ID NO: 36) | 1.1 | 1.0 |
| Biotin-1kPEG-rftaf (SEQ ID NO: 37) | 1.1 | 1.1 |

MFIR = Mean Fluorescence Intensity Ratio (relative to Biotin1k-PEG)

Of the 8 hits obtained from the CD8α/β screen, nfyrr (SEQ ID NO:32) and vlyrr (SEQ ID NO:30) show binding to immobilized recombinant CD8 protein. The EC₅₀ of vlyrr (SEQ ID NO:30) is 77±19 nM. The EC₅₀ of nfyrr (SEQ ID NO:32) is 47±8 nM. The data agree between the solid-phase ELISA and flow experiments.

Concentration of biotin-1kPEG-tfpkk (SEQ ID NO:21) and -nfyrr (SEQ ID NO:32) was titrated on SupT1 and Jurkat cells. The biotin-1kPEG-ligands were incubated with CD8+SupT1 cells and CD8−Jurkat cells at a concentrations of 10 mM or 20 mM. Increased binding of biotin-1kPEG-tfpkk (SEQ ID NO:21) and Biotin-1kPEG-nfyrr (SEQ ID NO:32) was observed by flow cytometry with increasing concentrations of the ligands.

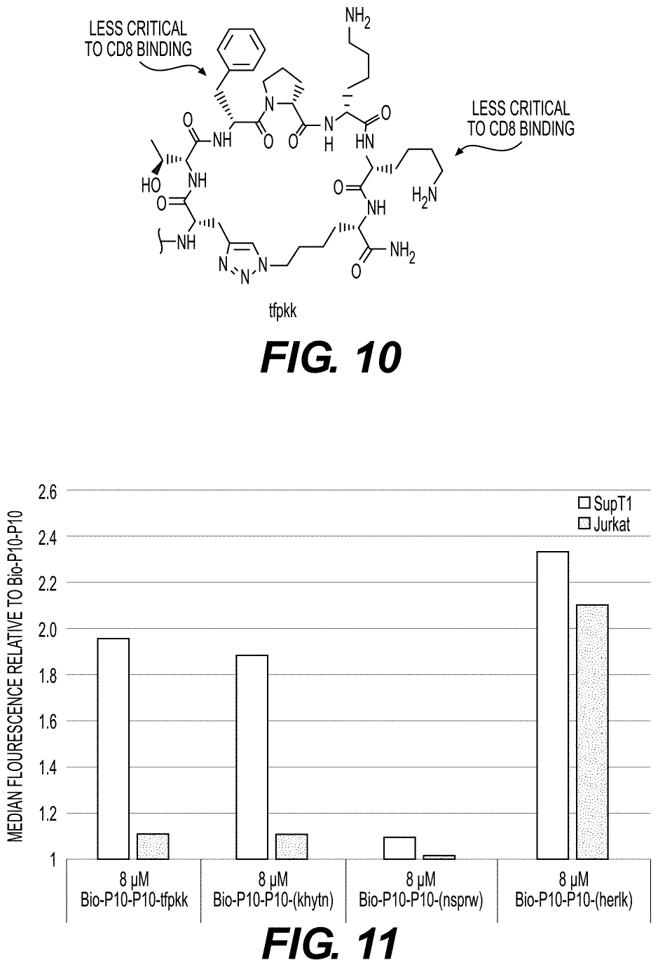

4-mers of tfpkk (SEQ ID NO:21) were prepared with deleted residues suggested by Ala scan. FIG. 10 shows full length tfpkk (SEQ ID NO:21) with the less critical residues (the tyrosine residue and the penultimate lysine residue) noted.

tfpkk (SEQ ID NO:32) linker modifications, using different combinations of PEG linkers, can be used to employ the most available PEGs while maintain the needed or desired linker length.

### Example 3

### Constrained PSMA Ligands

Constrained PSMA epitopes were produced by introducing cysteines into PSMA epitopes. Cross-linking of the cysteines limit the flexibility of the epitope and increasing their binding affinity due to the constraint. These epitopes were used for targeting in PCC screening are shown in Table 10. The bold, underlined amino acid is the amino acid replace with an amino acid that includes a click chemistry handle. Epitopes 6 and 7 have cysteines added at the ends to allow cross-linking.

TABLE 10

| | Epitope 2C | Epitope 6 | Epitope 7 |
|---|---|---|---|
| Sequence | [T498-G516] | [P231-G250] | [G311-F333] |
| | TKKSPSPEFSGM- | CPADYFAPGVKSYPDG- | CGSAPPDSSWRGSLKVPY- |
| | PRISKLG | WNLPGC | NVGPGFC |
| | SEQ ID NO: 77 | SEQ ID NO: 78 | SEQ ID NO: 79 |
| Total | 2424.995 Å² | 2310.988 Å² | 2791.497 Å² |
| Surface | | | |
| Area | | | |
| Solvent | 1307.707 Å² | 1053.848 Å² | 1379.497 Å² |
| Accessible | | | |
| Surface | | | |
| Area | | | |
| Net | +3 | -1 | +1 |
| Charge | | | |
| Backbone | 0.721 Å (0.156) | 1.338 Å (0.312) | 1.519 Å (0.347) |
| RMSD | | | |
| Side | 1.059 Å (0.153) | 1.588 Å (0.354) | 1.714 Å (0.352) |
| Chain | | | |
| RMSD | | | |

Epitopes 6 and 7 are cyclic epitopes that are cyclized using a disulfide linkage. While the inclusion of the bis-cysteines are not found naturally in the PSMA sequence, the conformational restrictions of this cyclic epitope can lead to a higher hit rate. Epitopes 6 and 7 also overlap the putative binding site of J591, the PSMA monoclonal antibody that is in the clinic. The click handles of these epitopes are separated by 13 Å, indicating that a biligand strategy is feasible. The N and C termini of both epitopes are stapled via a cysteine disulfide to reduce conformational flexibility of the synthetic epitope.

Related constrained epitopes are epitope 6b (GCPADYFAPGVKSYPDGWNLPGCG (SEQ ID NO:80)) and epitope 7b (GCGSAPPDSSWRGSLKVPYNVGPGFCG (SEQ ID NO:81)) (not yet used for screening). Epitope 6b is P231-G250 of PSMA, has a total surface area of 2310.988 Å², a solvent accessible surface of 1053.848 Å², a net charge of −1, average backbone RMSD of 1.338 (0.312), and average RMSD (including side chains) of 1.588 (0.354). Epitope 7b is G311-F333 of PSMA, has a total surface area of 2791.497 Å², a solvent accessible surface of 1379.497 Å², a net charge of +1, average backbone RMSD of 1.519 (0.347), and average RMSD (including side chains) of 1.714 (0.352).

### Example 4

### CD8 Ligands

CD8 ligands khytn (SEQ ID NO:82), nsprw (SEQ ID NO:83), and herlk (SEQ ID NO:84) were discovered by screening a peptide library with a cocktail of four CD8α epitopes: Epitope A2 Biotin-PEG_{S}-SQFRVSPLDRTW-[Az4]-LGETVELK[C→S]QVL (SEQ ID NO:93), Epitope B2 Biotin-PEGS-KCQVL-[Az4]-SNPTSGCSW (disulfide constrained) (SEQ ID NO:94), Epitope H Biotin-PEGS-RFSGKRL-[Az4]-DTFVLTLSD (SEQ ID NO:95), and Epitope J Biotin-PEG_{S}-QNKPKAAEG-[Az4]-DTQRF (SEQ ID NO:96).

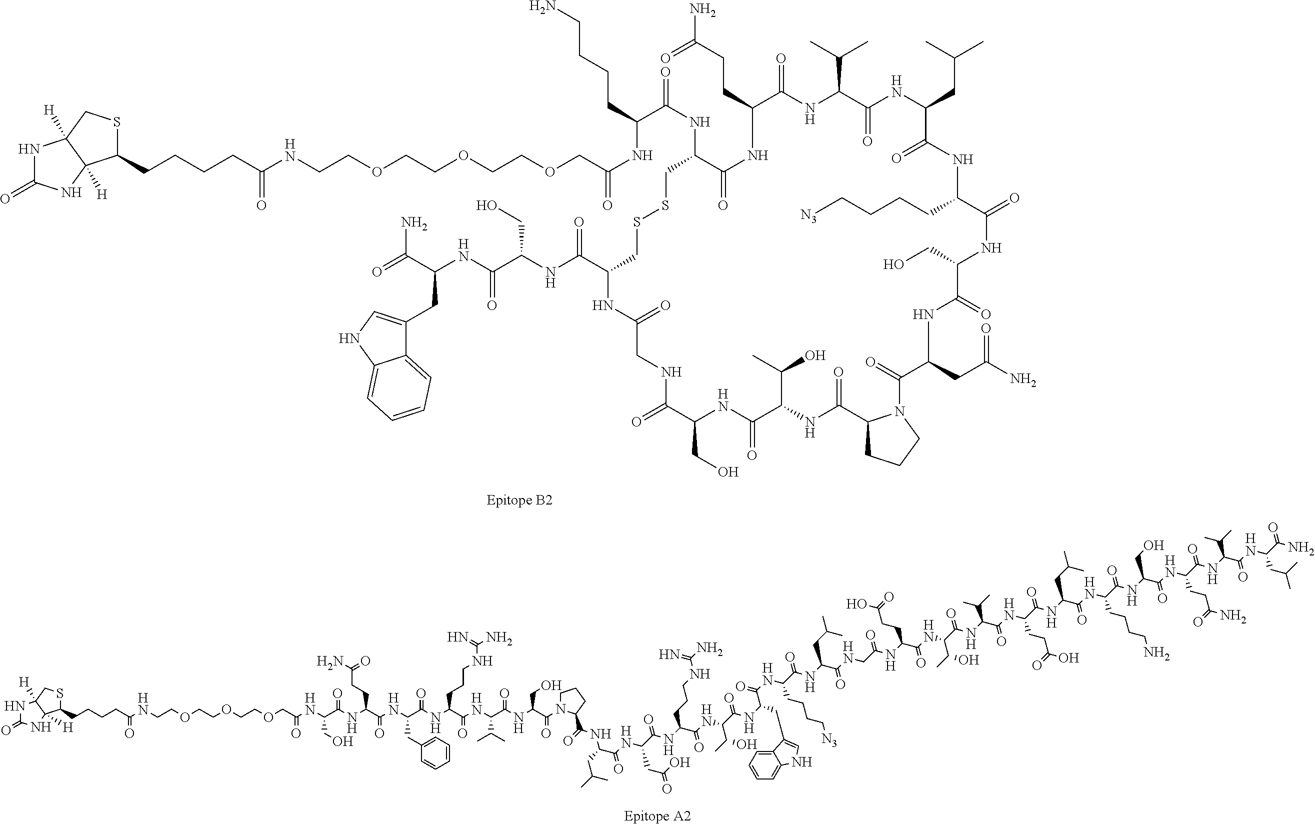

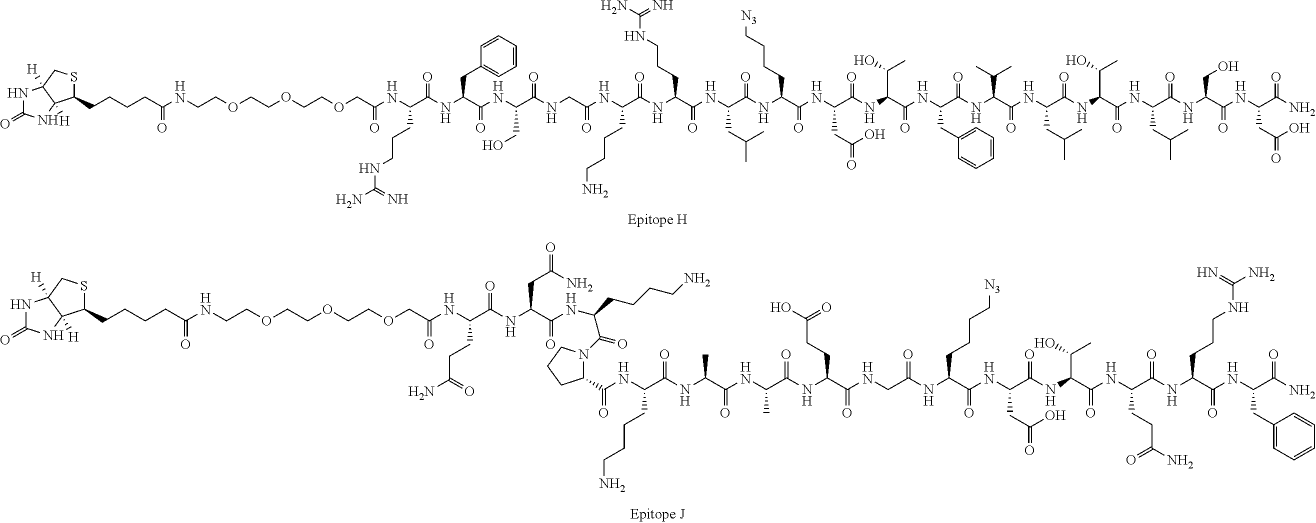

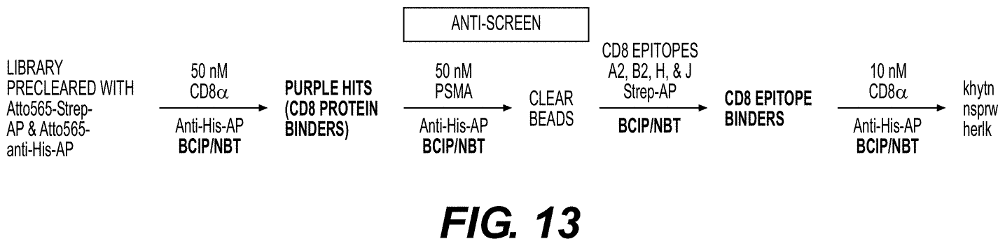

The screening (FIG. 13) involved pre-clearing an OBOC library with ATT0565-labeled Streptavidin-AP and anti-6×His-AP, primary screening against human-cell expressed His-CD8a (50 nM), anti-screening against human-cell expressed PSMA, secondary screening against a cocktail of the four CD8a epitopes (A2, B2, H, and J), and tertiary screening against human-cell expressed His-CD8a (10 nM). This improved screening method uses constrained epitopes (e.g., constrained epitope B2) to better mimic topology in the context of protein structure, incorporates new epitopes (e.g., modified epitope A2 and new epitopes H and J) to survey more of the CD8 extracellular domain, and uses more comprehensive pre-clear conditions and an anti-screen against an unrelated protein to remove non-specific hits during screen.

Three hits were left after this screening (Table 11). These hits were assessed in solid phase ELISA and in flow cytometry experiments.

TABLE 11
Hit peptides in four epitope cocktail screening

| x1 | x2 | x3 | x4 | x5 |
|---|---|---|---|---|
| k | h | y | t | n |
| n | s | p | r | w |
| h | e | r | l | k |

Epitope B2 ligand tfpkk (SEQ ID NO:21) and the three hit ligands selected against CD8 epitopes A2, B2, H, and J were tested for binding to CD8+ and CD8− cells by flow cytometry. The ligands tfpkk (SEQ ID NO:21) and khytn (SEQ ID NO:82) bind to epitope B2.

In flow cytometry, ligands tfpkk (SEQ ID NO:21) and khytn (SEQ ID NO:82) show similar selective binding to CD8+ cells, ligand nsprw (SEQ ID NO:83) shows weak (and weakly selective) binding to CD8+ cells, and ligand herlk (SEQ ID NO:84) shows weakly selective binding to CD8+ cells (FIG. 11). In ELISA, ligand khytn (SEQ ID NO:82) has similar binding affinity to CD8αβ (EC₅₀=6 μM) as ligand tfpkk (SEQ ID NO:21). The results show the power of constrained epitopes since the hit ligand khytn against constrained epitope B2 has superior binding characteristics.

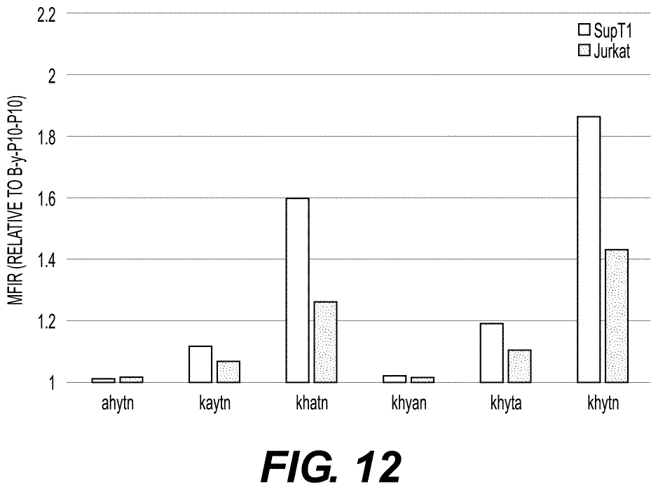

Ligand khytn (SEQ ID NO:82) was analyzed by alanine scanning. The alanine-substituted ligands were tested in flow cytometry to CD8+(SupT1) and CD8−(Jurkat) cells (FIG. 12). Substitution of D-tyrosine with Ala does not significantly change the selective binding of SupT1 and Jurkat, indicating that D-tyrosine is less critical for CD8 binding. Substitutions of D-histidine and D-asparagine are somewhat tolerated. Substitution of D-lysine or D-threonine is detrimental, indicating that these two amino acids are very important for CD8 binding. Thus, the D-tyrosine at position 3 can be replaced or modified without affecting the selective CD8 binding.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a ,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise.

Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different amino acids does not indicate that the listed amino acids are obvious one to the other, nor is it an admission of equivalence or obviousness.

Every peptide disclosed herein is intended to be and should be considered to be specifically disclosed herein. Further, every subgroup that can be identified within this disclosure is intended to be and should be considered to be specifically disclosed herein.

As a result, it is specifically contemplated that any peptide, or subgroup of peptides can be either specifically included for or excluded from use or included in or excluded from a list of peptides.

Those skilled in the art will recognize many equivalents to the specific embodiments of the method and compositions described herein. Such equivalents are intended to be encompassed by the following claims.

We claim:

1. A peptide comprising an epitope, wherein the peptide is cross-linked, wherein the epitope corresponds to an epitope of a target, wherein the peptide does not include the entire target, wherein the cross-link is a disulfide that naturally occurs in the target or between added or substitute cysteines.

2. The peptide of claim 1, wherein the target is a target protein.

3. The peptide of claim 2, wherein the target protein is CD8.

4. The peptide of claim 1, wherein the epitope comprises amino acids 24 to 33 of CD8.

5. The peptide of claim 1, wherein the disulfide is between amino acids C22 and C33 of CD8.

6. The peptide of claim 1, wherein the peptide comprises amino acids 21 to 35 of CD8.

7. The peptide of claim 1 further comprising an alkyne moiety, an azide moiety, a reporter moiety, or combinations thereof.

8. The peptide of claim 7, wherein the azide moiety and the alkyne moiety are comprised in an artificial amino acid.

9. The peptide of claim 8, wherein the artificial amino acid is propargylglycine (Fra).

10. The peptide of claim 7, wherein the reporter moiety is biotin.

11. The peptide of claim 1 further comprising an alkyne moiety, an azide moiety, a reporter moiety, or combinations thereof, wherein the epitope comprises a phosphorylated amino acid, wherein the azide moiety and the alkyne moiety of the target peptide are metalorganic molecules that selectively bind to the phospho group on the phosphorylated amino acid.

12. The peptide of claim 11, wherein the target is a target protein, wherein the target protein is CD8, wherein the phosphorylated amino acid is Ser235 or Ser236 of CD8.

13. The peptide of claim 11, wherein the metalorganic molecule comprises the reporter moiety.

14. The peptide of claim 13, wherein the reporter moiety is biotin.

15. A method of preparing a peptide comprising an epitope, wherein the epitope corresponds to an epitope of a target, wherein the peptide does not include the entire target, the method comprising cross-linking the peptide, wherein the cross-link is a disulfide that naturally occurs in the target or between added or substitute cysteines.

16. The method of claim 15, wherein the target is a target protein.

17. The method of claim 16, wherein the target protein is CD8.

18. The method of claim 15, wherein the epitope comprises amino acids 24 to 33 of CD8.

19. The method of claim 15, wherein the disulfide is between amino acids C22 and C33 of CD8.

20. The method of claim 15, wherein the peptide comprises amino acids 21 to 35 of CD8.

21. The method of claim 15, wherein the peptide further comprises an alkyne moiety, an azide moiety, a reporter moiety, or combinations thereof.

22. The method of claim 21, wherein the azide moiety and the alkyne moiety are comprised in an artificial amino acid.

23. The method of claim 22, wherein the artificial amino acid is propargylglycine (Fra).

24. The method of claim 21, wherein the reporter moiety is biotin.

25. The method of claim 15, wherein the peptide further comprises an alkyne moiety, an azide moiety, a reporter moiety, or combinations thereof, wherein the epitope comprises a phosphorylated amino acid, wherein the azide moiety and the alkyne moiety of the target peptide are metalorganic molecules that selectively bind to the phospho group on the phosphorylated amino acid.

26. The method of claim 25, wherein the target is a target protein, wherein the target protein is CD8, wherein the phosphorylated amino acid is Ser235 or Ser236 of CD8.

27. The method of claim 25, wherein the metalorganic molecule comprises the reporter moiety.

28. The method of claim 27, wherein the reporter moiety is biotin.

29. A method for identifying a target binding compound, the method comprising:
(A) contacting a first peptide library with a target peptide comprising an epitope,
wherein the first peptide library comprising a plurality of first peptide library members, wherein the first peptide library members optionally individually comprise an alkyne, azide, reporter moiety, or combinations thereof,
wherein the target peptide is cross-linked, wherein the epitope corresponds to an epitope of a target, wherein the target peptide does not include the entire target, wherein the cross-link is a disulfide that naturally occurs in the target or between added or substitute cysteines, wherein the target peptide optionally comprises an alkyne moiety, an azide moiety, a reporter moiety, or combinations thereof;
(B) identifying a first peptide library member with affinity for a first binding site on the epitope; and optionally:
(C) contacting a second peptide library with a composition comprising (i) the target or the target peptide and (ii) the first peptide library member of step B, wherein, prior to contacting, the first peptide library member of step B is modified to include an alkyne moiety or an azide moiety,
wherein the second peptide library comprises a plurality of second peptide library members, wherein the second peptide library members each comprise an azide moiety, an alkyne moiety, or both,
whereby a triazole-linked conjugate is formed between the first peptide library member of step B and a second peptide library member, whereby the triazole-linked second peptide library member is identified as having affinity for a second binding site on the target or the target peptide.

30. The method of claim 29, wherein the epitope is a distinct molecular surface of the target.

31. The method of claim 29, wherein the target peptide provides a catalytic scaffold for promoting the covalent coupling of the azide moiety and the alkyne moiety to form the triazole linkage.

32. The method of claim 29, wherein the azide moiety and the alkyne moiety of the first peptide library member of step B are comprised in an artificial amino acid.

33. The method of claim 32, wherein the artificial amino acid is propargylglycine (Fra).

34. The method of claim 29, wherein the epitope comprises a phosphorylated amino acid, wherein the azide moiety and the alkyne moiety of the target peptide are metalorganic molecules that selectively bind to the phospho group on the phosphorylated amino acid.

35. The method of claim 34, wherein the metalorganic molecule comprises the reporter moiety.

36. The method of claim 35, wherein the reporter moiety is biotin.

37. The method of claim 34, wherein the metalorganic molecule comprises an azide moiety.

38. The method of claim 29, wherein the reporter moiety is biotin.

39. The method of claim 29 further comprising:
(C) contacting a second peptide library with a composition comprising (i) the target or the target peptide and (ii) the first peptide library member of step B, wherein, prior to contacting, the first peptide library member of step B is modified to include an alkyne moiety or an azide moiety,
wherein the second peptide library comprises a plurality of second peptide library members, wherein the second peptide library members each comprise an azide moiety, an alkyne moiety, or both,
whereby a triazole-linked conjugate is formed between the first peptide library member of step B and a second peptide library member, whereby the triazole-linked second peptide library member is identified as having affinity for a second binding site on the target or the target peptide.

40. The method of claim 39 further comprising:
(D) contacting a third peptide library with a composition comprising (i) the target or the target peptide and (ii) the triazole-linked conjugate formed in step C, wherein, prior to contacting, the triazole-linked conjugate formed in step C is modified to include an alkyne moiety or an azide moiety,
wherein the third peptide library comprises a plurality of third peptide library members, wherein the third peptide library members each comprise an azide moiety, an alkyne moiety, or both,
whereby a triazole-linked conjugate is formed between the triazole-linked conjugate formed in step C and a third peptide library member, whereby the triazole-linked third peptide library member is identified as having affinity for a third binding site on the target or the target peptide.

41. The method of claim 40 further comprising:
(E) contacting a fourth peptide library with a composition comprising (i) the target or the target peptide and (ii) the triazole-linked conjugate formed in step D, wherein, prior to contacting, the triazole-linked conjugate formed in step D is modified to include an alkyne moiety or an azide moiety,
wherein the fourth peptide library comprises a plurality of fourth peptide library members, wherein the fourth peptide library members each comprise an azide moiety, an alkyne moiety, or both,
whereby a triazole-linked conjugate is formed between the triazole-linked conjugate formed in step D and a fourth peptide library member, whereby the triazole-linked fourth peptide library member is identified as having affinity for a fourth binding site on the target or the target peptide.

42. The method of claim 41 further comprising:
(F) contacting an nth peptide library with a composition comprising (i) the target or the target peptide and (ii) the triazole-linked conjugate formed in the immediately prior contacting step, wherein, prior to contacting, the triazole-linked conjugate formed in he immediately prior contacting step is modified to include an alkyne moiety or an azide moiety,
wherein the nth peptide library comprises a plurality of nth peptide library members, wherein the nth peptide library members each comprise an azide moiety, an alkyne moiety, or both,
whereby a triazole-linked conjugate is formed between the triazole-linked conjugate formed in the immediately prior contacting step and an nth peptide library member, whereby the triazole-linked nth peptide library member is identified as having affinity for an nth binding site on the target or the target peptide.

43. The method of claim 42 further comprising repeating step F one or more times.

44. The method of claim 29, wherein the first peptide library member of step B is identified by selecting a first peptide library member linked to the target peptide via a triazole linkage.

45. The method of claim 39, wherein the second peptide library member of step C is identified by selecting a second peptide library member linked to the first peptide library member via a triazole linkage.

46. The method of claim 40, wherein the third peptide library member of step D is identified by selecting a third peptide library member linked to the triazole-linked conjugate formed in step C via a triazole linkage.

47. The method of claim 41, wherein the fourth peptide library member of step E is identified by selecting a fourth peptide library member linked to the triazole-linked conjugate formed in step D via a triazole linkage.

48. The method of claim 42, wherein the nth peptide library member of step F is identified by selecting an nth peptide library member linked to the triazole-linked conjugate formed in the immediately prior contacting step via a triazole linkage.

49. The method of claim 44, wherein one or more or the triazole-linked peptide library members are selected by selecting a peptide library member labeled with the reporter moiety.

50. The method of claim 29, wherein the target is a target protein, wherein the method further comprises testing the first peptide library member of step B for binding to the target protein.

51. The method of claim 39, wherein the target is a target protein, wherein the method further comprises testing the triazole-linked conjugate formed in step C for binding to the target protein.

52. The method of claim 40, wherein the target is a target protein, wherein the method further comprises testing the triazole-linked conjugate formed in step D for binding to the target protein.

53. The method of claim 41, wherein the target is a target protein, wherein the method further comprises testing the triazole-linked conjugate formed in step E for binding to the target protein.

54. The method of claim 42, wherein the target is a target protein, wherein the method further comprises testing the triazole-linked conjugate formed in the immediately prior contacting step for binding to the target protein.

55. The method of claim 39, wherein the second peptide library is contacted with a composition comprising the target peptide and the first peptide library member of step B.

56. The method of claim 39, wherein the second peptide library is contacted with a composition comprising the target and the first peptide library member of step B.

57. The method of claim 40, wherein the third peptide library is contacted with a composition comprising the target peptide and the triazole-linked conjugate formed in step C.

58. The method of claim 40, wherein the third peptide library is contacted with a composition comprising the target and the triazole-linked conjugate formed in step C.

59. The method of claim 41, wherein the fourth peptide library is contacted with a composition comprising the target peptide and the triazole-linked conjugate formed in step D.

60. The method of claim 41, wherein the fourth peptide library is contacted with a composition comprising the target and the triazole-linked conjugate formed in step D.

61. The method of claim 42, wherein the nth peptide library is contacted with a composition comprising the target peptide and the triazole-linked conjugate formed in the immediately prior contacting step.

62. The method of claim 42, wherein the nth peptide library is contacted with a composition comprising the target and the triazole-linked conjugate formed in the immediately prior contacting step.

63. The method of claim 29, wherein the epitope is a distinct molecular surface of the target.

64. The method of claim 29, further comprising determining the peptide sequence of the first peptide library member of step B.

65. The method of claim 39, further comprising determining the peptide sequence of the second peptide library member of step C.

66. The method of claim 40, further comprising determining the peptide sequence of the third peptide library member of step D.

67. The method of claim 41, further comprising determining the peptide sequence of the fourth peptide library member of step E.

68. The method of claim 42, further comprising determining the peptide sequence of the nth peptide library member of step F.

69. The method of claim 64, wherein the peptide sequence of one or more of the peptide library members is determined by Edman degradation.

70. The method of claim 29, further comprising modifying the triazole linked conjugate formed in step C to contain a triazole or alkyne and contacting the modified conjugate with the target or the target peptide and a third peptide library, the third peptide library comprising a plurality of third peptide library members, each third peptide library member comprising an azide or alkyne, thereby forming a triazole linkage between the modified conjugate and a member of the third peptide library, wherein the third peptide library member is identified as having affinity for a third binding site on the target or the target peptide.

71. The method of claim 70, further comprising modifying the triazole linked conjugate formed between the modified conjugate and the member of the third peptide library to contain a triazole or alkyne and contacting the modified conjugate with the target or the target peptide and a fourth peptide library, the fourth peptide library comprising a plurality of fourth peptide library members, each fourth peptide library member comprising an azide or alkyne, thereby forming a triazole linkage between the modified conjugate and a member of the fourth peptide library, wherein the fourth peptide library member is identified as having affinity for a fourth binding site on the target or the target peptide.

72. The method of claim 71, further comprising modifying the triazole linked conjugate formed between the modified conjugate and the identified fourth peptide library member to contain a triazole or alkyne and contacting the modified conjugate with the target or the target peptide and an nth peptide library, the nth peptide library comprising a plurality of nth peptide library members, each nth peptide library member comprising an azide or alkyne, thereby forming a triazole linkage between the modified conjugate and a member of the nth peptide library, wherein the nth peptide library member is identified as having affinity for an nth binding site on the target or the target peptide.

73. The method of claim 72, further comprising:
(i) modifying the triazole linked conjugate formed between the modified conjugate and the identified nth peptide library member to contain a triazole or alkyne and
(ii) contacting the modified conjugate with the target or the target peptide and an n+1th peptide library, the n+1th peptide library comprising a plurality of N+1th peptide library members, each n+1th peptide library member comprising an azide or alkyne, thereby forming a triazole linkage between the modified conjugate and a member of the N+1th peptide library, the N+1th peptide library member having affinity for an n+1 th binding site on the target or the target peptide,
optionally repeating steps (i) and (ii) one or more times.

74. The method of claim 29, wherein the first binding site is an epitope.

75. The method of claim 29, wherein the second binding site is a second epitope.

76. The method of claim 40, wherein the third binding site is a third epitope.

77. The method of claim 40, wherein the fourth binding site is a fourth epitope.

78. The method of claim 29, wherein the target is a protein.

79. The method of claim 78, wherein the protein is an enzyme or cell surface protein.

80. The method of claim 79, wherein the protein is CD8.

81. The peptide of claim 1, wherein the target peptide comprises a linkage to a reporter moiety, wherein the reporter moiety comprises polyethylene glycol (PEG), biotin, thiol, a fluorophore, or combinations thereof.

82. The peptide of claim 81, wherein the fluorophore is carboxyfluorescein (FAM), fluorescein isothiocyanate (FITC), Cyanine-5 (Cy5), tetramethylrhodamine (TRITC) or Carboxytetramethylrhodamine (TAMRA).

83. A capture agent for CD8, the capture agent comprising a first ligand that specifically binds to a first epitope of CD8.

84. The capture agent of claim 83 further comprising a second ligand, wherein the first ligand and the second ligand are covalently linked to each other, wherein the second ligand specifically binds to a second epitope of CD8 that is distinct from the epitope to which the first ligand specifically binds.

85. The capture agent of claim 84, wherein the first and second epitopes are in different locations on CD8.

86. The capture agent of claim 84 further comprising a linker covalently connecting the first ligand to the second ligand.

87. The capture agent of claim 84, wherein the first ligand comprises an amino acid sequence 80-100% identical to an amino acid sequence selected from the group consisting of tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), and khyta (SEQ ID NO:89).

88. The capture agent of claim 84, wherein the first ligand comprises an amino acid sequence 80-100% identical to an amino acid sequence selected from the group consisting of hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), and teGwf (SEQ ID NO:20).

89. The capture agent of claim 84, wherein the first ligand comprises an amino acid sequence 80-100% identical to an amino acid sequence selected from the group consisting of krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), and hGrGh (SEQ ID NO:74).

90. The capture agent of claim 84, wherein the first ligand comprises an amino acid sequence 80-100% identical to an amino acid sequence selected from the group consisting of tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), khyta (SEQ ID NO:89), hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), teGwf (SEQ ID NO:20), krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), hGrGh (SEQ ID NO:74), nsprw (SEQ ID NO:83), and herlk (SEQ ID NO:84).

91. The capture agent of claim 84, wherein the first ligand comprises an amino acid sequence selected from the group consisting of tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), and khyta (SEQ ID NO:89).

92. The capture agent of claim 84, wherein the first ligand comprises an amino acid sequence selected from the group consisting of hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), and teGwf (SEQ ID NO:20).

93. The capture agent of claim 84, wherein the first ligand comprises an amino acid sequence selected from the group consisting of krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), and hGrGh (SEQ ID NO:74).

94. The capture agent of claim 84, wherein the first ligand comprises an amino acid sequence selected from the group consisting of tfpkk (SEQ ID NO:21), khytn (SEQ ID NO:82), khxtn (SEQ ID NO:91), kxytn (SEQ ID NO:91), khytx (SEQ ID NO:92), kaytn (SEQ ID NO:86), khatn (SEQ ID NO:87), khyta (SEQ ID NO:89), hsfvt (SEQ ID NO:9), kdnsn (SEQ ID NO:10), rtnnh (SEQ ID NO:11), eandr (SEQ ID NO:12), Glenr (SEQ ID NO:13), nnrvG (SEQ ID NO:14), eyeyv (SEQ ID NO:15), Gwdpn (SEQ ID NO:16), dwfsn (SEQ ID NO:17), kklwa (SEQ ID NO:18), wphtv (SEQ ID NO:19), teGwf (SEQ ID NO:20), krsah (SEQ ID NO:27), snprk (SEQ ID NO:28), nfpkk (SEQ ID NO:29), vlyrr (SEQ ID NO:30), yrpfy (SEQ ID NO:31), nfyrr (SEQ ID NO:32), yfrsr (SEQ ID NO:33), yrsny (SEQ ID NO:34), rpyay (SEQ ID NO:35), aykfn (SEQ ID NO:36), rftaf (SEQ ID NO:37), HGSYG (SEQ ID NO:38), KRLGA (SEQ ID NO:39), AKYRG (SEQ ID NO:40), hallw (SEQ ID NO:41), lrGyw (SEQ ID NO:42), vashf (SEQ ID NO:43), nGnvh (SEQ ID NO:44), wplrf (SEQ ID NO:45), rwfnv (SEQ ID NO:46), havwh (SEQ ID NO:47), wvplw (SEQ ID NO:48), ffrly (SEQ ID NO:49), wyyGf (SEQ ID NO:50), AGDSW (SEQ ID NO:51), HVRHG (SEQ ID NO:52), HGRGH (SEQ ID NO:53), THPTT (SEQ ID NO:54), FAGYH (SEQ ID NO:55), WTEHG (SEQ ID NO:56), PWTHG (SEQ ID NO:57), TNDFD (SEQ ID NO:58), LFPFD (SEQ ID NO:59), slrfG (SEQ ID NO:60), yfrGs (SEQ ID NO:61), wnwvG (SEQ ID NO:62), vaw1G (SEQ ID NO:63), fhvhG (SEQ ID NO:64), wvsnv (SEQ ID NO:65), wsvnv (SEQ ID NO:66), lnshG (SEQ ID NO:67), yGGvr (SEQ ID NO:68), nsvhG (SEQ ID NO:69), ttvhG (SEQ ID NO:70), fdvGh (SEQ ID NO:71), rhGwk (SEQ ID NO:72), Ghtwp (SEQ ID NO:73), hGrGh (SEQ ID NO:74), nsprw (SEQ ID NO:83), and herlk (SEQ ID NO:84).

95. The method of claim 15, wherein the epitope is 5 to 30 amino acids long, preferably the epitope is 8 to 20 amino acids long, more preferably the epitope is 7 to 13 amino acids long.

96. The method of claim 15, wherein the first epitope is, at most, 5, 6, 7, 8, 9, 10, 11, 12, 13, 14, 15, 16, 17, 18, 19 or 20 amino acids long.

97. The method of claim 15, wherein the first epitope comprises the amino acid sequence KCQVLLSNPTSGCSW (SEQ ID NO:1).

98. The method of claim 97, wherein L26 is replaced with an azide-containing amino acid residue.

99. The method of claim 15, wherein the first epitope comprises the amino acid sequence SQFRVSPLDRTWNLGETVELKSQVL (SEQ ID NO:97).

100. The method of claim 99, wherein N13 is replaced with an azide-containing amino acid residue.

101. The method of claim 15, wherein the first epitope comprises the amino acid sequence SQFRVSPLDR (SEQ ID NO:2).

102. The method of claim 101, wherein an azide-containing amino acid residue is added ahead of S1.

103. The method of claim 15, wherein the first epitope comprises the amino acid sequence VLLSNPTSGC (SEQ ID NO:3).

104. The method of claim 103, wherein an azide-containing amino acid residue is added ahead of V24.

105. The method of claim 15, wherein the first epitope comprises the amino acid sequence KAAEGLDTQRFSGKRLGDTF (SEQ ID NO:4).

106. The method of claim 105, wherein R67 is replaced with an azide-containing amino acid residue.

107. The method of claim 15, wherein the first epitope comprises the amino acid sequence FSGKRLGDTFVLTLSD (SEQ ID NO:5).

108. The method of claim 107, wherein G74 is replaced with an azide-containing amino acid residue.

109. The method of claim 15, wherein the first epitope comprises the amino acid sequence SALSNSIMYFSHFVPV (SEQ ID NO:6).

110. The method of claim 109, wherein M102 is replaced with an azide-containing amino acid residue.

111. The method of claim 15, wherein the first epitope comprises the amino acid sequence RFSGKRLGDTFVLTLSD (SEQ ID NO:98.

112. The method of claim 111, wherein G74 is replaced with an azide-containing amino acid residue.

113. The method of claim 15, wherein the first epitope comprises the amino acid sequence QNKPKAAEGLDTQRF (SEQ ID NO:99).

114. The method of claim 113, wherein L63 is replaced with an azide-containing amino acid residue.

115. The method of claim 15, wherein the first epitope comprises the amino acid sequence FQPRGAAASPTFL (SEQ ID NO:7).

116. The method of claim 15, wherein the first epitope comprises the amino acid sequence LYLSQNKPKAA (SEQ ID NO:8).
